(19) United States
(12) Patent Application Publication
(10) Pub. No.: US 2018/0271890 A1
(43) Pub. Date: Sep. 27, 2018

(54) Solid Pharmaceutical Composition Comprising Amorphous Sofosbuvir

(71) Applicant: Sandoz AG, Basel (CH)

(72) Inventors: Franz Xaver SCHWARZ, Kundl (AT); Nolwenn MARTIN, Kundl (AT)

(73) Assignee: Sandoz AG, Basel (CH)

(21) Appl. No.: 15/763,649

(22) PCT Filed: Oct. 6, 2016
(86) PCT No.: PCT/EP2016/073912

§ 371 Date: Mar. 27, 2018

(30) Foreign Application Priority Data
Oct. 7, 2015 (EP) 15188787.4
Aug. 12, 2016 (EP) 16184053.3

(51) Int. Cl. A61K 31/7072; A61K 9/14; A61K 9/16; A61K 9/20; A61K 47/32; A61P 31/14

(52) U.S. Cl. CPC A61K 31/7072; A61K 9/146; A61K 9/1635; A61K 9/19; A61K 47/32; A61P 31/14; A61K 9/2054

(57) ABSTRACT

A solid pharmaceutical composition comprising a solid composition, wherein the solid com-position comprises sofosbuvir and at least one pharmaceutically acceptable matrix compound, wherein at least 99 weight-% of the sofosbuvir comprised in the solid composition are present in amorphous form, at least 99 weight-% of the solid composition consist of the sofosbuvir and the at least one matrix compound, wherein the solid composition contains the sofosbuvir in an amount of at least 25 weight-%, or at least 30 weight-%, or at least 35 weight-%, or at least 40 weight-% or at least 50 weight-% or at least 55 weight-% or preferably at least 5 weight-% based on the combined weight of the sofosbuvir and the at least one matrix com-pound, wherein in the adsorption-desorption isotherm of the at least one pharmaceutically acceptable matrix compound, the desorption mass difference minus the adsorption mass dif-ference at 75% relative humidity and 25° C. is less than 0.

The present invention relates to a solid pharmaceutical composition comprising amorphous sofosbuvir and a process for the preparation of the solid pharmaceutical composition. Further, the present invention relates to the use of the solid pharmaceutical composition for the treatment of hepatitis C.

Sofosbuvir according to formula (I)

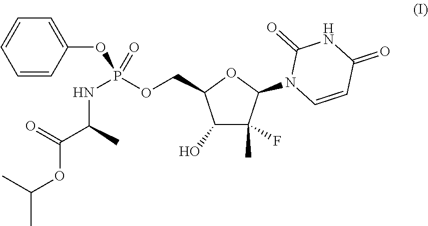 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

with IUPAC name (S)-isopropyl 2-(((S)-(((2R,3R,4R,5R)-5-(2,4-dioxo-3,4-dihydropyrimidin-1(2H)-yl)-4-fluoro-3-hydroxy-4-methyltetrahydrofuran-2-yl)methoxy)(phenoxy)phosphoryl)-amino)propanoate is a drug inhibiting the RNA polymerase used by the hepatitis C virus to replicate its RNA.

In WO 2010/135569 A, sofosbuvir is described as a moisture unstable compound. In particular, it was found that under stress conditions at 40° C. and a relative humidity (RH) of 75%, sofosbuvir deliquesces after a few hours. Amorphous sofosbuvir, compared to its crystalline forms, is even less moisture stable and deliquesces at a relative humidity above about 50%. On the other hand, compared to its crystalline forms, amorphous sofosbuvir is believed to show a higher solubility when applied to a patient.

Among many other drugs, WO 2013/101550 A describes sofosbuvir, referred to as PSI-7977. In particular, this document relates to a theoretical assessment tool allegedly useful to rank the intrinsic physical stability of amorphous drug substances. As parameter which indicates the physical stability, the crystallization tendency is mentioned. Without giving any details regarding the specific type of drug, WO 2013/101550 A discloses allegedly stable compositions which may contain from 1 to 50% by weight of the drug wherein, however, the drug content is preferably in the range of from 5 to 15% by weight. Not one single actual example directed to a concrete composition which would have been subjected to a respective stability test is disclosed in WO 2013/101550 A. Still further, theoretical examples according to WO 2013/101550 A directed to HCV inhibitors in general teach a very low drug content of only 10% by weight.

Therefore, the problem underlying the present invention is the provision of a stable pharmaceutical composition comprising amorphous sofosbuvir which contains a high amount of the sofosbuvir.

A very promising approach to solve this problem could be the provision of a solid composition from which a solid pharmaceutical composition is prepared, wherein in this solid composition, sofosbuvir is stable in its amorphous form when subjected to stress conditions of a temperature of 40° C. and a relative humidity of 75%, and wherein this solid composition comprises the sofosbuvir in a high amount well above 50 weight-%. Such stable solid compositions may be characterized in that they essentially consist of sofosbuvir and at least one pharmaceutically acceptable matrix compound, wherein the matrix compound in turn is characterized in that in its adsorption-desorption isotherm, the mass difference Δm(desorption) at 75% relative humidity and 25° C. is greater than or equal to, preferably greater than, the mass difference Δm(adsorption) at 75% relative humidity and 25° C., determined according to dynamic vapor sorption measurement.

However, surprisingly, it was found that this problem can be solved if a solid pharmaceutical composition is prepared based on a solid composition such as a solid dispersion comprising sofosbuvir, wherein it is not necessary that the solid composition itself is stable in terms of the stability of the amorphous form of sofosbuvir comprised in the solid composition when subjected to stress conditions such as a temperature of 40° C. and a relative humidity of 75%. In particular, it was found that this problem can be solved if a solid pharmaceutical composition is prepared based on a solid composition such as a solid dispersion comprising sofosbuvir and at least one pharmaceutically acceptable matrix compound, wherein said matrix compound does not exhibit the adsorption-desorption isotherm characteristics mentioned above.

Therefore, the present invention relates to a solid pharmaceutical composition comprising a solid composition, wherein the solid composition comprises sofosbuvir according to formula (I)

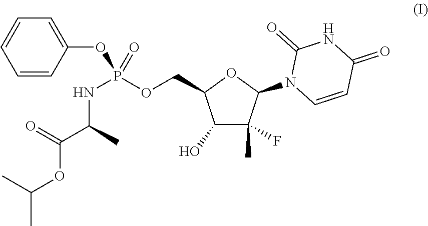 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

and at least one pharmaceutically acceptable matrix compound, wherein at least 99 weight-% of the sofosbuvir comprised in the solid composition are present in amorphous form, at least 99 weight-% of the solid composition consist of the sofosbuvir and the at least one matrix compound, wherein the solid composition contains the sofosbuvir in an amount of at least 10 weight %, or at least 15 weight %, or at least 20 weight %, or at least 25 weight-%, or at least 30 weight-%, or at least 35 weight-%, or at least 40 weight-% or at least 50 weight-% or preferably at least 55 weight-% based on the combined weight of the sofosbuvir and the at least one matrix compound,
wherein in the adsorption-desorption isotherm of the at least one pharmaceutically acceptable matrix compound, ΔΔm/% which is defined as the mass difference Δm(desorption)/% at 75% relative humidity and 25° C. minus the mass difference Δm(adsorption)/% at 75% relative humidity and 25° C., determined according to dynamic vapor sorption measurement, in particular determined according to dynamic vapor sorption measurement as described in Reference Example 1 herein, is <0.

Further, the present invention relates to a process for preparing a solid pharmaceutical composition, preferably the solid pharmaceutical composition described above, comprising
(i) preparing a solid composition comprising sofosbuvir and at least one pharmaceutically acceptable matrix compound, wherein at least 99 weight-% of the sofosbuvir comprised in the solid composition are present in amorphous form, at least 99 weight-% of the solid composition consist of the sofosbuvir and the at least one matrix compound, wherein the solid composition contains the sofosbuvir in an amount of at least 25 weight-%, or at least 30 weight-%, or at least 35 weight-%, or at least 40 weight-% or at least 50 weight-% or preferably at least 55 weight-% based on the combined weight of the sofosbuvir and the at least one matrix compound,
(ii) processing the solid composition obtained from (i) to the pharmaceutical composition;
   wherein in the adsorption-desorption isotherm of the at least one pharmaceutically acceptable matrix compound, ΔΔm/% which is defined as the mass difference Δm(desorption)/% at 75% relative humidity and 25° C. minus the mass difference Δm(adsorption)/% at 75% relative humidity and 25° C., determined according to dynamic vapor sorption measurement, in particular determined according to dynamic vapor sorption measurement as described in Reference Example 1 herein, is <0.

Preferably, the solid composition according to the present invention contains the sofosbuvir in an amount of at least 25 weight-%, or at least 30 weight-%, or at least 35 weight-%, or at least 40 weight-% or at least 50 weight-% or at least 55 weight-%. Preferably, the amount of sofosbuvir is at least 55 weight-%. It is further preferred that amount of sofosbuvir is in the range of from 55 to 95 weight-%, more preferably in the range of from 65 to 90 weight-%, based on the combined weight of the sofosbuvir and the at least one matrix compound. More preferably, the solid composition contains the sofosbuvir in an amount in the range of from 70 to 85 weight-%, based on the combined weight of the sofosbuvir and the at least one matrix compound. Preferred ranges of the sofosbuvir content of the solid composition are from 70 to 75 weight-% or from 75 to 80 weight-% or 80 to 85 weight-%, based on the combined weight of the sofosbuvir and the at least one matrix compound. An especially preferred range is from 80 to 85 weight-%.

Compared to the teaching of the prior art, in particular the teaching of WO 2013/101550 A, the present invention thus provides the possibility to provide compositions having a high sofosbuvir content which allow to administer the sofosbuvir to a patient in need thereof with only a few or even only one dosage. Further in particular with regard to dosage forms such as tablets, these high sofosbuvir contents allow to prepare smaller tablets which can be swallowed easily by the patient. Yet further, it is not necessary to take into account the stability of amorphous sofosbuvir in the solid composition since it was found that in the solid pharmaceutical composition, sofosbuvir is stable in its amorphous form.

### Solid Pharmaceutical Composition

According to the present invention, ΔΔm/% of the matrix compound is less than 0.

With regard to ΔΔm/% of the matrix compound, preferred values are in the range of from −1.0≤ΔΔm/%<0, preferably in the range of from −0.9≤ΔΔm/%<0, more preferably in the range of from −0.8≤ΔΔm/%<0, more preferably in the range of from −0.7≤ΔΔm/%<0, more preferably in the range of from −0.6≤ΔΔm/%<0. Further, preferred values are in the range of from −1.0≤ΔΔm/%≤0.001, preferably in the range of from −0.9≤ΔΔm/%≤0.001, more preferably in the range of from −0.8≤ΔΔm/%≤0.001, more preferably in the range of from −0.7≤ΔΔm/%≤0.001, more preferably in the range of from −0.6≤ΔΔm/%≤0.001.

Further preferably, ΔΔm/% of the matrix compound is in the range of from −0.5≤ΔΔm/%<0, preferably in the range of from −0.4≤ΔΔm/%<0, more preferably in the range of from −0.3≤ΔΔm/%<0, preferably in the range of from −0.2≤ΔΔm/%<0. Further, preferred values are in the range of from −0.5≤ΔΔm/%≤0.001, preferably in the range of from −0.4≤ΔΔm/%≤0.001, more preferably in the range of from −0.3≤ΔΔm/%≤0.001, more preferably in the range of from −0.2≤ΔΔm/%≤0.001.

More preferably, ΔΔm/% of the matrix compound is in the range of from −0.1≤ΔΔm/%<0, more preferably in the range of from −0.1≤ΔΔm/%≤0.001, more preferably in the range of from −0.1≤ΔΔm/%≤0.01.

Regarding the dynamic vapor sorption measurements and the determination of the values of Δm(desorption) and Δm(adsorption) at 75% relative humidity and 25° C., specific reference is made to Reference Example 1 of the present invention.

According to the present invention, at least 99 weight-% of the sofosbuvir comprised in the solid composition are present in amorphous form. Preferably, at least 99.5 weight-%, more preferably at least 99.6 weight-%, more preferably at least 99.7 weight-%, more preferably at least 99.8 weight-%, more preferably at least 99.9 weight-% of the sofosbuvir comprised in the solid composition are present in amorphous form. More preferably, at least 99.95 weight-%, more preferably at least 99.99 weight-% of the sofosbuvir comprised in the solid composition are present in amorphous form. The term “amorphous form” as used in this context of the present invention relates to sofosbuvir which, subjected to X-ray powder diffraction spectroscopy, does not contain any detectable crystalline form.

According to the present invention, at least 99 weight-% of the solid composition consist of the sofosbuvir and the at least one matrix compound. Preferably, at least 99.5 weight-%, more preferably at least 99.6 weight-%, more preferably at least 99.7 weight-%, more preferably at least 99.8 weight-%, more preferably at least 99.9 weight-% of the solid composition consist of the sofosbuvir and the at least one matrix compound. More preferably, at least 99.95 weight-%, more preferably at least 99.99 weight-% of the solid composition consist of the sofosbuvir and the at least one matrix compound.

Certain compositions disclosed in WO 2013/101550 A which may be regarded as examples and which describe compositions comprising 10% by weight of a drug different from sofosbuvir contain a surfactant, namely vitamin E TPGS, sorbitan monolaurate, propylene glycol monocarpylate, or a combination of vitamin E TGPS and lauryl glycol FCC. These surfactants are disclosed to be present in the compositions in very significant amounts of 7 weight-%, based on the total weight of the compositions. Thus, it appears that WO 2013/101550 A, in its most concrete embodiments, teaches the mandatory use of surfactants in significant amounts if a physically stable composition is to be provided. Surprisingly, for the solid compositions of the present invention comprising sofosbuvir, it was found that no such surfactant is necessary to ultimately provide a physically stable solid pharmaceutical composition. Therefore, the present invention also relates to the above-described solid pharmaceutical composition, wherein the solid composition comprised in the solid pharmaceutical composition comprises less than 0.1 weight-%, preferably less than 0.01 weight-%, more preferably less than 0.001 weight-%, more preferably less than 0.0001 weight-%, more preferably in the range of from 0 to 0.00001 weight-% of vitamin E TPGS (D-alpha-tocopheryl polyethylene glycol 1000 succinate), or of sorbitan monolaurate, or of a combination of vitamin E TGPS and lauryl glycol FCC. Preferably, the present invention relates to the above-described solid pharmaceutical composition, wherein the solid composition comprised in the solid pharmaceutical composition comprises less than 0.1 weight-%, preferably less than 0.01 weight-%, more preferably less than 0.001 weight-%, more preferably less than 0.0001 weight-%, more preferably in the range of from 0 to 0.00001 weight-% of polysorbate 20, or of polysorbate 40, or of polysorbate 60, or of polysorbate 80, or of Cremophor RH 40, or of Cremophor EL, or of Gelucire 44/14, or of Gelucire 50/13, or of vitamin E TPGS, or of propylene glycol laurate, or of sodium lauryl sulfate, or of sorbitan monolaurate, or of a combination or a mixture of two or more thereof. More preferably, the present invention relates to the above-described solid pharmaceutical composition, wherein the solid composition comprised in the solid pharmaceutical composition comprises less than 0.1 weight-%, preferably less than 0.01 weight-%, more preferably less than 0.001 weight-%, more preferably less than 0.0001 weight-%, more preferably in the range of from 0 to 0.00001 weight-% of polyoxyethylene castor oil derivatives, e.g. polyoxyethyleneglycerol triricinoleate or polyoxyl 35 castor oil (Cremophor EL; BASF Corp.) or polyoxyethyleneglycerol oxystearate such as polyethylenglycol 40 hydrogenated castor oil (Cremophor RH 40, also known as polyoxyl 40 hydrogenated castor oil or macrogolglycerol hydroxystearate) or polyethylenglycol 60 hydrogenated castor oil (Cremophor RH 60); or a mono fatty acid ester of polyoxyethylene sorbitan, such as a mono fatty acid ester of polyoxyethylene (20) sorbitan, e.g. polyoxyethylene (20) sorbitan monooleate (Tween 80), polyoxyethylene (20) sorbitan monostearate (Tween 60), polyoxyethylene (20) sorbitan monopalmitate (Tween 40), or polyoxyethylene (20) sorbitan monolaurate (Tween 20), or polyoxyethylene alkyl ethers, e.g. polyoxyethylene (3) lauryl ether, polyoxyethylene (5) cetyl ether, polyoxyethylene (2) stearyl ether, polyoxyethylene (5) stearyl ether; or polyoxyethylene alkylaryl ethers, e.g. polyoxyethylene (2) nonylphenyl ether, polyoxyethylene (3) nonylphenyl ether, polyoxyethylene (4) nonylphenyl ether, polyoxyethylene (3) octylphenyl ether; or polyethylene glycol fatty acid esters, e.g. PEG-200 monolaurate, PEG-200 dilaurate, PEG-300 dilaurate, PEG-400 dilaurate, PEG-300 distearate, PEG-300 dioleate; alkylene glycol fatty acid mono esters, e.g. propylene glycol monolaurate (lauroglycol, such as lauroglycol FCC); or sucrose fatty acid esters, e.g. sucrose monostearate, sucrose distearate, sucrose monolaurate, sucrose dilaurate; or sorbitan fatty acid mono esters such as sorbitan mono laurate (Span 20), sorbitan monooleate, sorbitan monopalmitate (Span 40), or sorbitan stearate; or D-alpha-tocopheryl polyethylene glycol 1000 succinate; or a combination or mixture thereof; or block copolymers of ethylene oxide and propylene oxide, also known as polyoxyethylene polyoxypropylene block copolymers or polyoxyethylene polypropyleneglycol, such as Poloxamer 124, Poloxamer 188, Poloxamer 237, Poloxamer 388, or Poloxamer 407, or a combination of two or more thereof. More preferably, the present invention relates to the above-described solid pharmaceutical composition, wherein the solid composition comprised in the solid pharmaceutical composition comprises less than 0.1 weight-%, preferably less than 0.01 weight-%, more preferably less than 0.001 weight-%, more preferably less than 0.0001 weight-%, more preferably in the range of from 0 to 0.00001 weight-% of a pharmaceutically acceptable surfactant having an HLB value of from 2-20. More preferably, the present invention relates to the above-described solid pharmaceutical composition, wherein the solid composition comprised in the solid pharmaceutical composition comprises less than 0.1 weight-%, preferably less than 0.01 weight-%, more preferably less than 0.001 weight-%, more preferably less than 0.0001 weight-%, more preferably in the range of from 0 to 0.00001 weight-% of a pharmaceutically acceptable non-ionic surfactant. More preferably, the present invention relates to the above-described solid pharmaceutical composition, wherein the solid composition comprised in the solid pharmaceutical composition comprises less than 0.1 weight-%, preferably less than 0.01 weight-%, more preferably less than 0.001 weight-%, more preferably less than 0.0001 weight-%, more preferably in the range of from 0 to 0.00001 weight-% of a pharmaceutically acceptable surfactant. In each case, the weight-% values are based on the total weight of the solid composition.

Regarding the at least one pharmaceutically acceptable matrix compound, it was surprisingly found that matrix compounds which exhibit specific characteristics when subjected to a dynamic vapor sorption measurement are suitable as matrix compounds according to the present invention although the resulting solid composition, in contrast to the solid pharmaceutical composition, is not stable in terms of the stability of the amorphous form of sofosbuvir. In particular, it was found that these matrix compounds do not stabilize amorphous sofosbuvir in the solid composition but, once processed to a solid pharmaceutical composition, do stabilize amorphous sofosbuvir in the solid pharmaceutical composition according to the present invention, even at high sofosbuvir contents. The amorphous sofosbuvir in the pharmaceutical composition of the invention does not crystalize nor deliquescence.

Therefore, the present invention relates to the solid pharmaceutical composition described above, having a moisture stability of at least 95%, preferably at least 98%, more preferably at least 99%, wherein the moisture stability is defined as the amount of solid amorphous sofosbuvir which is present in the solid pharmaceutical composition after having been exposed to a relative humidity of 75% at 40° C. for 8 weeks, relative to the amount of solid amorphous sofosbuvir which is present in the solid pharmaceutical composition before said exposure. The term “before said exposure” as used in this context of the present application relates to a solid pharmaceutical composition which, prior to being exposed to a relative humidity of 75% at 40° C., has been stored, directly after its preparation, at a relative humidity of 30% at 25° C. Therefore, the present invention also relates to the solid pharmaceutical composition described above, having a moisture stability of at least 95%, preferably at least 98%, more preferably at least 99%, wherein the moisture stability is defined as the amount of solid amorphous sofosbuvir which is present in the solid pharmaceutical composition after having been exposed to a relative humidity of 75% at 40° C. for 8 weeks, relative to the amount of solid amorphous sofosbuvir which is present in the solid pharmaceutical composition when, directly after its preparation, being stored at a relative humidity of 30% at 25° C. Regarding the at least one pharmaceutically acceptable matrix compound, it was found that in particular hydrophilic polymers, preferably hydrophilic water-soluble polymers, and silicon-based inorganic adsorbents are suitable matrix compounds. Preferably, the at least one matrix compound is selected from the group consisting of hydrophilic water-soluble polymers, silicon-based inorganic adsorbents and a combination of two or more thereof. For example, the at least one matrix compound is selected from the group consisting of hydrophilic polymers, preferably hydrophilic water-soluble polymers, and combinations of two or more thereof; or from the group consisting of silicon-based inorganic adsorbents and combinations of two or more thereof; or from the group consisting of combinations of at least one hydrophilic polymer, preferably hydrophilic water-soluble polymer, and at least one silicon-based inorganic adsorbent.

Examples of hydrophilic polymers include, but are not restricted to, polysaccharides, preferably cellulose derivatives, polyvinylpyrrolidones, polyethylene glycols, polyethylene glycol based copolymers, polyacrylic acids, salts of polyacrylic acids, polyvinyl alcohols, polyacrylamide copolymers, methacrylic acid copolymers, methacrylate copolymers, pectines, chitin derivatives, chitosan derivatives, polyphosphates, polyoxazolines, and mixtures of two or more thereof. More specific examples of hydrophilic polymers include, but are not restricted to, cellulose derivatives selected from the group consisting of alkylcellulose, preferably methylcellulose, ethylcellulose, or propylcellulose; hydroxalkylcellulose, preferably hydroxymethylcellulose, hydroxyethylcellulose, or hydroxypropylcellulose (HPC) such as Klucel® LF; hydroxyalkylalkylcellulose, preferably hydroxyethylmethylcellulose (HEMC), or hydroxypropylmethylcellulose (HPMC); carboxyalkylcellulose, preferably carboxymethylcellulose (CMC), carboxymethylhydroxyethylcellulose (CMHEC), hydroxyethylcarboxymethylcellulose (HECMC); sodium carboxymethylcellulose, cellulose acetate phthalate (CAP), hydroxypropylmethylcellulose acetate (HPMCA), hydroxypropylmethylcellulose phthalate (HPMCP), hydroxypropylmethylcellulose acetate succinate (HPMCAS), and a mixture of two or more thereof, and polyvinylpyrrolidones (PVP, polyvidone, povidone) such as PVP 40, vinyl pyrrolidone-based copolymers such as vinyl pyrrolidone-vinyl acetate copolymer like copovidone, and other polymers such as polyethylene glycol, polyvinyl acetate and polyvinylcaprolactame-based graft copolymer like Soluplus®.

Examples of silicon-based inorganic adsorbents include, but are not restricted to, silica, silicates, and a combination of two or more thereof. For example, the silicon-based inorganic adsorbent is selected from the group consisting of silicas and combinations of two or more thereof; or from the group consisting of silicates and combinations of two or more thereof; or from the group consisting of at least one silica and at least one silicate. The term “silicate” as used in this context of the present invention refers to naturally occurring or synthesized compounds containing an anionic silicon compound, preferably an oxide. Examples of such silicates include, but are not restricted to, nesosilicates comprising the structure unit [SiO₄]⁴⁻, sorosilicates comprising the structure unit [Si₂O₇]⁶⁻, cyclosilicates comprising the structure unit [SiₙO₃ₙ]²ⁿ⁻, single chain inosilicates comprising the structure unit [SiₙO₃ₙ]²ⁿ⁻, double chain inosilicates comprising the structure unit [Si₄ₙO₁₁ₙ]⁶ⁿ⁻, phyllosilicates comprising the structure unit [SiₙO₅ₙ]²ⁿ⁻, or tectosilicates with a 3D framework comprising the structure unit [AlₓSi_{y}O_{2(x+y)}]^{x-}. The term “silica” as used in this context of the present invention refers to naturally occurring or synthesized silica. Examples of such silica include, but are not restricted to fumed silica, precipitated silica, gel silica, colloidal silica, such as Syloid® AL-1 FP.

As described above, the solid composition according to the present invention comprises at least one hydrophilic, preferably water-soluble, polymer and/or at least one silicon-based inorganic adsorbent. Generally, it is possible that the solid composition contains at least one hydrophilic, preferably water-soluble, polymer and at least one silicon-based inorganic adsorbent. Preferably, the solid composition of the present invention comprises either at least one hydrophilic, preferably water-soluble, polymer or at least one silicon-based inorganic adsorbent. Preferably, the solid composition of the present invention comprises, as matrix compound, one, two, or three, preferably one or two, more preferably one hydrophilic, preferably water-soluble, polymer(s). More preferably, the at least one matrix compound comprises at least one hydrophilic, preferably water-soluble, polymer, more preferably consists of at least one, more preferably one, hydrophilic, preferably water-soluble, polymer.

Preferably, the at least one hydrophilic, preferably water-soluble, polymer has a solubility in water of at least 10 g/l, more preferably of at least 15 g/l, more preferably of at least 20 g/l, more preferably of at least 25 g/l, more preferably of at least 30 g/l, in each case at 23° C. at atmospheric pressure.

Preferably, the weight average molecular weight (M_{w}) of the at least one hydrophilic water-soluble polymer is in the range of from 20 to 100 kDa, preferably in the range of from 30 to 85 kDa, more preferably in the range of from 40 to 75 kDa.

Preferably, the at least one hydrophilic water-soluble polymer comprises, preferably consists of, at least one vinyl pyrrolidone-vinyl acetate copolymer. More preferably, the at least one hydrophilic water-soluble polymer comprises, preferably consists of, copovidone. Copovidone is commercially available, such as under the tradename Kollidon® VA 64 or Kollidon® VA 64 Fine.

Therefore, the present invention also relates to a solid pharmaceutical composition comprising a solid composition, wherein the solid composition comprises sofosbuvir according to formula (I)

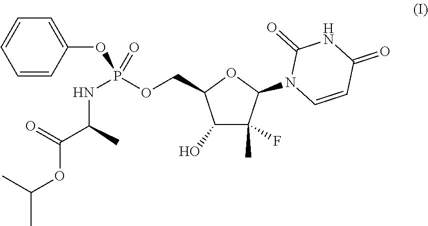 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

and at least one pharmaceutically acceptable matrix compound, wherein at least 99 weight-% of the sofosbuvir comprised in the solid composition are present in amorphous form, at least 99 weight-% of the solid composition consist of the sofosbuvir and the at least one matrix compound, wherein the solid composition contains the sofosbuvir in an amount of at least 25 weight-%, or at least 30 weight-%, or at least 35 weight-%, or at least 40 weight-% or at least 50 weight-% or preferably at least 55 weight-% based on the combined weight of the sofosbuvir and the at least one matrix compound, wherein the at least one matrix compound comprises, preferably consists of, at least one vinyl pyrrolidone-vinyl acetate copolymer, preferably copovidone. It is preferred that the solid composition contains the sofosbuvir in an amount of at least 55 weight-% based on the combined weight of the sofosbuvir and the at least one matrix compound.

Therefore, the present invention also relates to a solid pharmaceutical composition comprising a solid composition, wherein the solid composition comprises sofosbuvir according to formula (I)

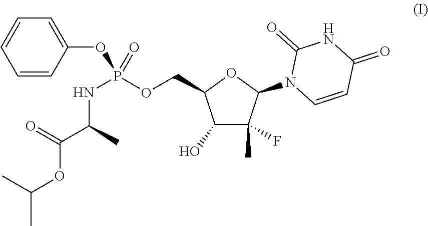 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

and at least one pharmaceutically acceptable matrix compound, wherein at least 99 weight-% of the sofosbuvir comprised in the solid composition are present in amorphous form, at least 99 weight-% of the solid composition consist of the sofosbuvir and the at least one matrix compound, wherein the solid composition contains the sofosbuvir in an amount of at least 25 weight-%, or at least 30 weight-%, or at least 35 weight-%, or at least 40 weight-% or at least 50 weight-% or preferably at least 55 weight-% based on the combined weight of the sofosbuvir and the at least one matrix compound, wherein the at least one matrix compound comprises, preferably consists of, at least one vinyl pyrrolidone-vinyl acetate copolymer, preferably copovidone;
said solid pharmaceutical composition having a moisture stability of at least 95%, preferably at least 98%, more preferably at least 99%, wherein the moisture stability is defined as the amount of solid amorphous sofosbuvir which is present in the solid pharmaceutical composition after having been exposed to a relative humidity of 75% at 40° C. for 8 weeks, relative to the amount of solid amorphous sofosbuvir which is present in the solid pharmaceutical composition before said exposure, said moisture stability preferably being determined as described in Reference Example 2 herein.

Preferably, the solid composition is a solid dispersion. The term “solid dispersion” as used in this context of the present invention relates to a composition in a solid state, i.e. a state which is neither liquid nor gaseous, wherein the amorphous sofosbuvir is dispersed in at least one of the at least one pharmaceutically acceptable matrix compounds comprised in the solid dispersion, preferably in all of the at least pharmaceutically acceptable one matrix compounds comprised in the solid dispersion.

Preferably, the solid pharmaceutical composition of the present invention is an oral dosage form, including, but not restricted to, a granule, a capsule, for example a capsule filled with granules, a sachet, a pellet, a dragee, a lozenge, a troche, a pastille, or a tablet, such as an uncoated tablet, a coated tablet, an effervescent tablet, a soluble tablet, a dispersible tablet, an orodispersible tablet, a tablet for use in the mouth, a chewable tablet or an extrudate. More preferably, the oral dosage form of the present invention is a tablet.

Preferably, the solid pharmaceutical composition, preferably the tablet of the present invention contains, in addition to the solid composition, at least one pharmaceutically acceptable excipient. Any pharmaceutically acceptable excipient can be employed as long as it does not detrimentally affect the properties of the solid pharmaceutical composition. Preferably, at least one of the at least one pharmaceutically acceptable excipient is different from the at least one pharmaceutically acceptable matrix compound comprised in the solid composition. More preferably, the at least one pharmaceutically acceptable excipient is different from the at least one pharmaceutically acceptable matrix compound comprised in the solid composition.

Therefore, the present invention also relates to a solid pharmaceutical composition comprising a solid composition, wherein the solid composition comprises sofosbuvir according to formula (I)

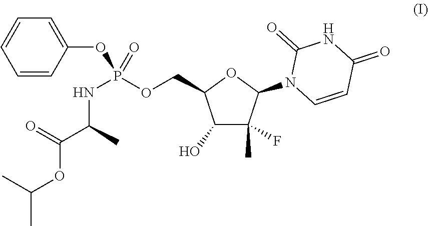 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

and at least one pharmaceutically acceptable matrix compound, wherein at least 99 weight-% of the sofosbuvir comprised in the solid composition are present in amorphous form, at least 99 weight-% of the solid composition consist of the sofosbuvir and the at least one matrix compound, wherein the solid composition contains the sofosbuvir in an amount of at least 25 weight-%, or at least 30 weight-%, or at least 35 weight-%, or at least 40 weight-% or at least 50 weight-% or preferably at least 55 weight-% based on the combined weight of the sofosbuvir and the at least one matrix compound, wherein the at least one matrix compound comprises, preferably consists of, at least one vinyl pyrrolidone-vinyl acetate copolymer, preferably copovidone;
wherein the solid pharmaceutical composition contains, in addition to the solid composition, at least one pharmaceutically acceptable excipient, which is not the at least one vinyl pyrrolidone-vinyl acetate copolymer comprised in the solid composition, preferably not copovidone.

Therefore, the present invention also relates to a solid pharmaceutical composition comprising a solid composition, wherein the solid composition comprises sofosbuvir according to formula (I)

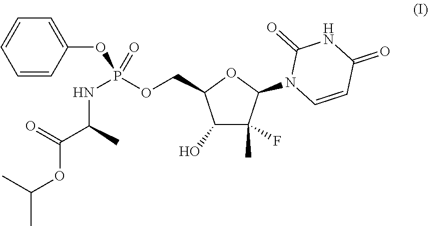 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

and at least one pharmaceutically acceptable matrix compound, wherein at least 99 weight-% of the sofosbuvir comprised in the solid composition are present in amorphous form, at least 99 weight-% of the solid composition consist of the sofosbuvir and the at least one matrix compound, wherein the solid composition contains the sofosbuvir in an amount of at least 55 weight-% based on the combined weight of the sofosbuvir and the at least one matrix compound, wherein the at least one matrix compound comprises, preferably consists of, at least one vinyl pyrrolidone-vinyl acetate copolymer, preferably copovidone;
wherein the solid pharmaceutical composition contains, in addition to the solid composition, at least one pharmaceutically acceptable excipient, which is not the at least one vinyl pyrrolidone-vinyl acetate copolymer comprised in the solid composition, preferably not copovidone.

Examples of generally conceivable pharmaceutically acceptable excipients comprise carriers such as solid carriers like magnesium carbonate, magnesium stearate, talc, sugar, lactose, pectin, dextrin, starch, gelatin, tragacanth, methycellulose, sodium carboxy-methylcellulose and wax; or liquid carriers such as water, aqueous or non-aqueous liquids, vehicles, diluents, solvents, binders, adjuvants, solubilizers, thickening agents, stabilizers, disintegrants, glidants, lubricating agents, buffering agents, emulsifiers, wetting agents, suspending agents, sweetening agents, colorants, flavors, coating agents, preservatives, antioxidants, processing agents, drug delivery modifiers, additives to make solutions isotonic, antifoaming agents, encapsulating material, surfactants, opacifing agents, enhancers, waxes, cap anti-locking agents (e.g. glycerol) and ion exchange resins. Other conceivable pharmaceutically acceptable additives are described in Remington's Pharmaceutical Sciences, 15^{th} edition, Mack Publishing Co., New Jersey (1991). The terms “pharmaceutically acceptable excipient” and “pharmaceutical excipient” as used in this context of the present invention refer to a compound that is used to prepare a pharmaceutical composition, and is generally safe, non-toxic and neither biologically nor otherwise undesirable, and includes excipients that are acceptable for veterinary use as well as human pharmaceutical use.

Preferably, the at least one pharmaceutically acceptable excipient includes one or more of at least one of a diluent, at least one disintegrant, at least one glidant, at least one lubricant, and combinations of two or more thereof. More preferably, the at least one pharmaceutically acceptable excipient comprises a combination of at least one a diluent, at least one disintegrant, at least one glidant, and at least one lubricant.

The at least one diluent preferably includes one or more of calcium carbonate, dicalcium phosphate, dry starch, calcium sulfate, cellulose, compressible sugars, confectioner's sugar, dextrates, dextrin, dextrose, dibasic calcium phosphate dihydrate, glyceryl palmitostearate, hydrogenated vegetable oil, inositol, kaolin, lactose, magnesium carbonate, magnesium oxide, maltodextrin, mannitol, microcrystalline cellulose, polymethacrylates, potassium chloride, powdered cellulose, powdered sugar, pregelatinized starch, sodium chloride, sorbitol, starch, sucrose, sugar spheres, talc, tribasic calcium phosphate.

The at least one disintegrant preferably includes one or more of agar, alginic acid, bentonite, carboxymethylcellulose calcium, carboxymethylcellulose sodium, carboxymethylcellulose, cellulose, a cation exchange resin, cellulose, gums, citrus pulp, colloidal silicon dioxide, corn starch, croscarmellose sodium, crospovidone, guar gum, hydrous aluminum silicate, an ion exchange resin such as polyacrin potassium, magnesium aluminum silicate, methyl cellulose, microcrystalline cellulose, modified cellulose gum, modified corn starch, montmorillonite clay, natural sponge, polyacrilin potassium, potato starch, powdered cellulose, povidone, pregelatinized starch, sodium alginate, sodium bicarbonate optionally in admixture with one or more acidulants, sodium starch glycolate, starch, silicates.

The at least one glidant preferably includes one or more of colloidal silicon dioxide, talc, starch, starch derivatives.

The at least one lubricant preferably includes one or more of calcium stearate, glyceryl monostearate, glyceryl palmitostearate, hydrogenated castor oil, hydrogenated vegetable oil, light mineral oil, magnesium stearate, mineral oil, polyethylene glycol, sodium benzoate, sodium lauryl sulfate, sodium stearyl fumarate, stearic acid, talc, zinc stearate.

Preferably, the at least one pharmaceutically acceptable excipient is a combination of at least one a diluent, at least one disintegrant, at least one glidant, and at least one lubricant. More preferably, the at least one pharmaceutically acceptable excipient is a combination of a diluent, a disintegrant, a glidant, and a lubricant. More preferably, the at least one pharmaceutically acceptable excipient comprises, preferably is, a combination of mannitol, microcrystalline cellulose, croscarmellose sodium, colloidal silica, magnesium stearate.

Therefore, the present invention also relates to a solid pharmaceutical composition comprising a solid composition, wherein the solid composition comprises sofosbuvir according to formula (I)

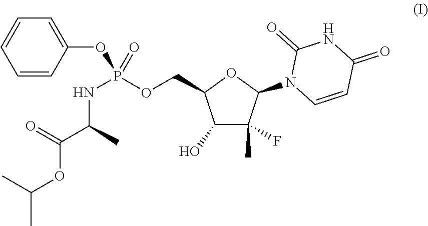 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

and at least one pharmaceutically acceptable matrix compound, wherein at least 99 weight-% of the sofosbuvir comprised in the solid composition are present in amorphous form, at least 99 weight-% of the solid composition consist of the sofosbuvir and the at least one matrix compound, wherein the solid composition contains the sofosbuvir in an amount of at least 25 weight-%, or at least 30 weight-%, or at least 35 weight-%, or at least 40 weight-% or at least 50 weight-% or preferably at least 55 weight-% based on the combined weight of the sofosbuvir and the at least one matrix compound, wherein the at least one matrix compound comprises, preferably consists of, at least one vinyl pyrrolidone-vinyl acetate copolymer, preferably copovidone;
wherein the solid pharmaceutical composition contains, in addition to the solid composition, at least one pharmaceutically acceptable excipient, which is not the at least one vinyl pyrrolidone-vinyl acetate copolymer comprised in the solid composition, preferably not copovidone, and wherein the at least one pharmaceutically acceptable excipient comprises, preferably is, a combination of a diluent, a disintegrant, a glidant, and a lubricant, preferably a combination of mannitol, microcrystalline cellulose, croscarmellose sodium, colloidal silica, magnesium stearate.

Therefore, the present invention also relates to a solid pharmaceutical composition comprising a solid composition, wherein the solid composition comprises sofosbuvir according to formula (I)

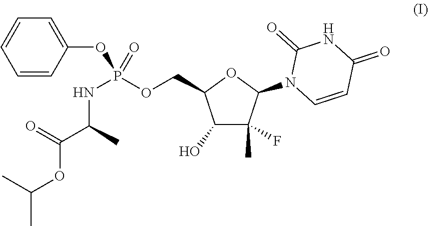 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

and at least one pharmaceutically acceptable matrix compound, wherein at least 99 weight-% of the sofosbuvir comprised in the solid composition are present in amorphous form, at least 99 weight-% of the solid composition consist of the sofosbuvir and the at least one matrix compound, wherein the solid composition contains the sofosbuvir in an amount of at least 55 weight-% based on the combined weight of the sofosbuvir and the at least one matrix compound, wherein the at least one matrix compound comprises, preferably consists of, at least one vinyl pyrrolidone-vinyl acetate copolymer, preferably copovidone;
wherein the solid pharmaceutical composition contains, in addition to the solid composition, at least one pharmaceutically acceptable excipient, which is not the at least one vinyl pyrrolidone-vinyl acetate copolymer comprised in the solid composition, preferably not copovidone, and wherein the at least one pharmaceutically acceptable excipient comprises, preferably is, a combination of a diluent, a disintegrant, a glidant, and a lubricant, preferably a combination of mannitol, microcrystalline cellulose, croscarmellose sodium, colloidal silica, magnesium stearate.

Generally, there is no specific restriction concerning the amount of the at least one pharmaceutically acceptable excipient which is comprised in the solid pharmaceutical composition. Generally, from 1 to 95 weight-% or from 2 to 90 weight-% or from 5 to 85 weight-% or from 10 to 80 weight-% or from 15 to 75 weight-% or from 20 to 70 weight-% or from 25 to 65 weight-% of the solid pharmaceutical composition consist of the solid composition. Preferably, from 30 to 65, more preferably from 35 to 55, more preferably from 40 to 45 weight-% of the solid pharmaceutical composition consist of the solid composition. More preferably, from 40 to 42 weight-% of the solid pharmaceutical composition consist of the solid composition.

Therefore, the present invention also relates to a solid pharmaceutical composition comprising a solid composition, wherein the solid composition comprises sofosbuvir according to formula (I)

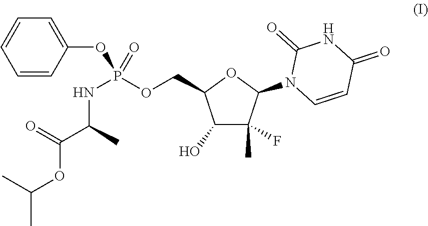 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

and at least one pharmaceutically acceptable matrix compound, wherein at least 99 weight-% of the sofosbuvir comprised in the solid composition are present in amorphous form, at least 99 weight-% of the solid composition consist of the sofosbuvir and the at least one matrix compound, wherein the solid composition contains the sofosbuvir in an amount of at least 25 weight-%, or at least 30 weight-%, or at least 35 weight-%, or at least 40 weight-% or at least 50 weight-% or preferably at least 55 weight-% based on the combined weight of the sofosbuvir and the at least one matrix compound, wherein the at least one matrix compound comprises, preferably consists of, copovidone;
wherein the solid pharmaceutical composition contains, in addition to the solid composition, at least one pharmaceutically acceptable excipient which is a combination of mannitol, microcrystalline cellulose, croscarmellose sodium, colloidal silica, magnesium stearate, and wherein from 40 to 45 weight-%, preferably from 40 to 42 weight-% of the solid pharmaceutical composition consist of the solid composition.

Therefore, the present invention also relates to a solid pharmaceutical composition comprising a solid composition, wherein the solid composition comprises sofosbuvir according to formula (I)

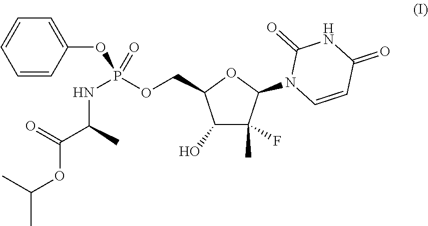 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

and at least one pharmaceutically acceptable matrix compound, wherein at least 99 weight-% of the sofosbuvir comprised in the solid composition are present in amorphous form, at least 99 weight-% of the solid composition consist of the sofosbuvir and the at least one matrix compound, wherein the solid composition contains the sofosbuvir in an amount of at least 55 weight-% based on the combined weight of the sofosbuvir and the at least one matrix compound, wherein the at least one matrix compound comprises, preferably consists of, copovidone;
wherein the solid pharmaceutical composition contains, in addition to the solid composition, at least one pharmaceutically acceptable excipient which is a combination of mannitol, microcrystalline cellulose, croscarmellose sodium, colloidal silica, magnesium stearate, and wherein from 40 to 45 weight-%, preferably from 40 to 42 weight-% of the solid pharmaceutical composition consist of the solid composition.

Further, it is preferred that the solid pharmaceutical composition comprises from 15 to 25 weight-%, preferably from 20 to 22 weight-% mannitol, from 25 to 35 weight-%, preferably from 30 to 32 weight-% microcrystalline cellulose, from 2 to 10 weight-%, preferably from 4 to 6 weight-% croscarmellose sodium, from 0.2 to 2 weight-%, preferably from 0.5 to 1.5 weight-% colloidal silica, from 0.5 to 5 weight-%, preferably from 1 to 1.5 weight-% magnesium stearate, in each based on the total weight of the solid pharmaceutical composition. More preferably, the solid pharmaceutical composition comprises from 20 to 21 weight-% mannitol, from 30 to 31 weight-% microcrystalline cellulose, from 4.5 to 5.5 weight-% croscarmellose sodium, from 0.5 to 1.0 weight-% colloidal silica, from 1.0 to 2.0 weight-% magnesium stearate, in each based on the total weight of the solid pharmaceutical composition.

Therefore, the present invention also relates to a solid pharmaceutical composition comprising a solid composition, wherein the solid composition comprises sofosbuvir according to formula (I)

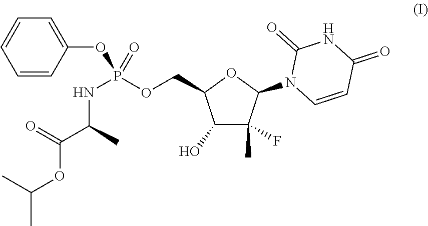 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

and at least one pharmaceutically acceptable matrix compound, wherein at least 99 weight-% of the sofosbuvir comprised in the solid composition are present in amorphous form, at least 99 weight-% of the solid composition consist of the sofosbuvir and the at least one matrix compound, wherein the solid composition contains the sofosbuvir in an amount of at least 25 weight-%, or at least 30 weight-%, or at least 35 weight-%, or at least 40 weight-% or at least 50 weight-% or preferably at least 55 weight-%, preferably from 75 to 85 weight-%, based on the combined weight of the sofosbuvir and the at least one matrix compound, wherein the at least one matrix compound comprises, preferably consists of, copovidone;
wherein the solid pharmaceutical composition contains, in addition to the solid composition, at least one pharmaceutically acceptable excipient which is a combination of mannitol, microcrystalline cellulose, croscarmellose sodium, colloidal silica, magnesium stearate, and wherein the solid pharmaceutical composition comprises from 40 to 41 weight-% of the solid composition, from 20 to 21 weight-% mannitol, from 30 to 31 weight-% microcrystalline cellulose, from 4.5 to 5.5 weight-% croscarmellose sodium, from 0.5 to 1.0 weight-% colloidal silica, from 1.0 to 2.0 weight-% magnesium stearate, in each based on the total weight of the solid pharmaceutical composition, wherein the individual contents add up to 100%.

Therefore, the present invention also relates to a solid pharmaceutical composition comprising a solid composition, wherein the solid composition comprises sofosbuvir according to formula (I)

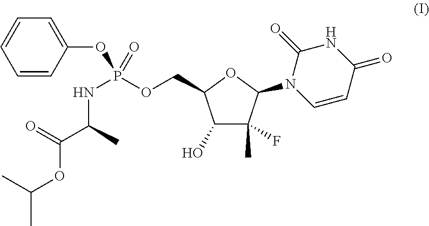 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

and at least one pharmaceutically acceptable matrix compound, wherein at least 99 weight-% of the sofosbuvir comprised in the solid composition are present in amorphous form, at least 99 weight-% of the solid composition consist of the sofosbuvir and the at least one matrix compound, wherein the solid composition contains the sofosbuvir in an amount of at least 55 weight-%, preferably from 75 to 85 weight-%, based on the combined weight of the sofosbuvir and the at least one matrix compound, wherein the at least one matrix compound comprises, preferably consists of, copovidone;
wherein the solid pharmaceutical composition contains, in addition to the solid composition, at least one pharmaceutically acceptable excipient which is a combination of mannitol, microcrystalline cellulose, croscarmellose sodium, colloidal silica, magnesium stearate, and wherein the solid pharmaceutical composition comprises from 40 to 41 weight-% of the solid composition, from 20 to 21 weight-% mannitol, from 30 to 31 weight-% microcrystalline cellulose, from 4.5 to 5.5 weight-% croscarmellose sodium, from 0.5 to 1.0 weight-% colloidal silica, from 1.0 to 2.0 weight-% magnesium stearate, in each based on the total weight of the solid pharmaceutical composition, wherein the individual contents add up to 100%.

Conceivably, the pharmaceutical composition of the present invention, in particular in form of a tablet, may further comprise a coating agent which may further comprise a taste-masking agent. The coating agent may be formed from an aqueous film coat composition, wherein the aqueous film coat composition may comprise a film-forming polymer, water and/or an alcohol as a vehicle, and optionally one or more adjuvants such as are known in the film-coating art. The coating agent may be selected from among hydroxypropylmethylcellulose, hydroxypropylcellulose, methylcellulose, ethylcellulose, hydroxyethylcellulose, cellulose acetate phthalate, sodium ethyl cellulose sulfate, carboxymethyl cellulose, polyvinylpyrrolidone, zein, and an acrylic polymer such as methacrylic acid/methacrylic acid ester copolymers such as methacrylic acid/methylmethacrylate copolymers, etc., and a polyvinyl alcohol. With respect to the coating agent, film-forming polymers are typically provided in either aqueous or organic solvent-based solutions or aqueous dispersions. The polymers may be also provided in dry form, alone or in a powdery mixture with other components such as a plasticizer and/or a colorant, which may be made into a solution or dispersion. The aqueous film coat composition may further comprise water as a vehicle for the other components. The vehicle may optionally further comprise one or more water soluble solvents, such as an alcohol and/or a ketone. Conceivable examples of an alcohol include but are not limited to methanol, isopropanol, propanol, etc. A non-limiting example for the ketone may be acetone. Suitable aqueous film coating compositions may include those commercially available from Colorcon, Inc. of West Point, Pa., under the trade name OPADRY and OPADRY II.

It is further contemplated, according to the present invention, that the solid pharmaceutical composition comprising, in addition to the sofosbuvir, one or more further anti-HCV agents. The anti-HCV agent is preferably ledipasvir according to formula (II)

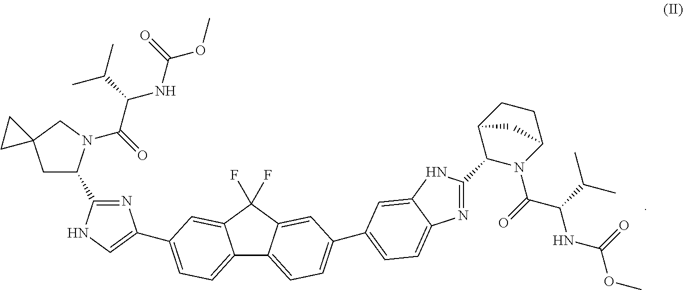 [SMILES: COC(=O)N[C@H](C(=O)N1CC2(CC2)C[C@H]1c1nc(-c2ccc3c(c2)C(F)(F)c2cc(-c4ccc5nc([C@@H]6[C@H]7CC[C@H](C7)N6C(=O)[C@@H](NC(=O)OC)C(C)C)[nH]c5c4)ccc2-3)c[nH]1)C(C)C]

Hence, it is contemplated that the solid pharmaceutical composition of the invention, comprising the solid composition and at least one pharmaceutically acceptable excipient, wherein more preferably, the solid pharmaceutical composition consists of the solid composition, the at least one pharmaceutically acceptable excipient and optionally the one or more further anti-HCV agents wherein preferably the anti-HCV agent is ledipasvir.

### Process for Preparing the Solid Pharmaceutical Composition

Generally, the solid pharmaceutical composition of the present invention can be prepared according to all suitable processes. Preferably, it is prepared by a process comprising
(i) preparing a solid composition comprising sofosbuvir and at least one pharmaceutically acceptable matrix compound, wherein at least 99 weight-% of the sofosbuvir comprised in the solid composition are present in amorphous form, at least 99 weight-% of the solid composition consist of the sofosbuvir and the at least one matrix compound, wherein the solid composition contains the sofosbuvir in an amount of at least 25 weight-%, or at least 30 weight-%, or at least 35 weight-%, or at least 40 weight-% or at least 50 weight-% or preferably at least 55 weight-% based on the combined weight of the sofosbuvir and the at least one matrix compound,
(ii) processing the solid composition obtained from (i) to the pharmaceutical composition; wherein in the adsorption-desorption isotherm of the at least one pharmaceutically acceptable matrix compound, ΔΔm/% which is defined as the mass difference Δm(desorption)/% at 75% relative humidity and 25° C. minus the mass difference Δm(adsorption)/% at 75% relative humidity and 25° C., determined according to dynamic vapor sorption measurement, in particular determined according to dynamic vapor sorption measurement as described in Reference Example 1 herein, is <0.

It is especially preferred that according to (ii), the solid composition obtained from (i) is directly processed to the pharmaceutical composition. The term “directly” as used in this context refers to a process wherein, after step (i) from which the solid composition is obtained and wherein at least 99%, preferably at least 99.5%, more preferably at least 99.9% of the sofosbuvir comprised in said solid composition are present in amorphous form, said solid composition is not subjected to any modifications prior to (ii). To ensure that the solid composition obtained from (i) is not subjected to any modifications prior to (ii), it is possible, for example, to subject the solid composition to the further processing according to (ii) immediately after step (i), wherein the solid composition obtained from (i) is processed to the pharmaceutical composition according to (ii) preferably at most 168 h, more preferably at most 72 h, more preferably at most 24 h after having been obtained from (i), wherein during this period of time, the solid composition is preferably not subjected to stress conditions of 40° C. and a relative humidity of 75%, more preferably stored under ambient conditions. Further, ensure that the solid composition obtained from (i) is not subjected to any modifications prior to (ii), it is possible, for example, to store the solid composition obtained from (i) under suitable conditions which prevent the solid composition from undergoing any modification.

No specific restrictions exist with regard to (i). Preferably, (i) comprises embedding the sofosbuvir in a matrix comprising, preferably consisting of the at least one pharmaceutically acceptable matrix compound, wherein the weight ratio of the sofosbuvir relative to the at least one matrix compound is at least 5.5:4.5, preferably in the range of from 5.5:4.5 to 9.5:0.5, more preferably in the range of from 6.5:3.5 to 9.0:1.0, more preferably in the range of from 7.5:2.5 to 8.5:1.5.

Regarding said embedding, it is preferred, on the one hand, that (i) comprises embedding the sofosbuvir in a matrix comprising, preferably consisting of the at least one pharmaceutically acceptable matrix compound, wherein said embedding comprises melting the at least one pharmaceutically acceptable matrix compound together with the sofosbuvir. Said melting is preferably realized by a hot melt method, more preferably by a hot-melt extrusion. The temperatures under which said melting is carried out is preferably in the range of from 75 to 175° C., more preferably in the range of from 80 to 165° C., more preferably in the range of from 85 to 160° C. more preferably in the range of from 90 to 150° C. After said melting process, the molten mixture, preferably the extrudate, is cooled, preferably to a temperature in the range of from 10 to 40° C., more preferably in the range of from 15 to 35° C., more preferably in the range of from 20 to 30° C.

The sofosbuvir which is employed into (i) as starting material is not subject to any restrictions. Preferably, the sofosbuvir in solid form is sofosbuvir in at least one crystalline form or in amorphous form or as a mixture of at least one crystalline form and amorphous form, wherein preferably at least 95 weight-%, preferably at least 99 weight-%, more preferably at least 99.9 weight-% of the sofosbuvir are present in at least one crystalline form.

On the other hand, it is preferred that (i) comprises preparing a solution of the sofosbuvir and the at least one pharmaceutically acceptable matrix compound in at least one solvent preferably selected from the group consisting of water, an organic solvent, and a combination of two or more thereof, more preferably selected from the group consisting of a C1-C2 halogenated hydrocarbon, a C1-C4 alcohol, a C3-C6 ketone, a C2-C6 ether, a C3-C5 ester, and a combination of two or more thereof, wherein said solution is preferably subjected to drying, preferably by lyophilizing the solution or spray-drying the solution.

According to the present invention, the solution of the sofosbuvir which is used as starting material for the preparation of the solid composition can be prepared according to all conceivable means. For example, the solution can be prepared from amorphous sofosbuvir, from crystalline sofosbuvir which is present in one or more crystalline forms, from a sofosbuvir salt, from a sofosbuvir solvate, from a sofosbuvir hydrate, or from a combination of two or more thereof. For example, it is possible to start from a solution prepared from crystalline sofosbuvir which is present in crystalline form 1. The preparation of crystalline form 1 of sofosbuvir is described, for example, in WO 2011/123645 A. Further, it is possible to start from a solution prepared from amorphous sofosbuvir. Therefore, the present invention also relates to a process as described above, wherein the solution of the sofosbuvir in at least one solvent is prepared from sofosbuvir of which at least 95 weight-%, preferably at least 99 weight-%, more preferably at least 99.9 weight-% are present in its amorphous form. No specific restrictions exist how the amorphous sofosbuvir is prepared. Generally, the amorphous sofosbuvir can be prepared from sofosbuvir which is present in at least one crystalline form or in amorphous form or as a mixture of at least one crystalline form and amorphous form. Preferably, the amorphous sofosbuvir is prepared from sofosbuvir of which at least 95 weight-%, preferably at least 99 weight-%, more preferably at least 99.9 weight-% are present in at least one crystalline form, such as in crystalline form 1. Generally, the crystalline and/or amorphous sofosbuvir is subjected to a melt method, preferably a hot-melt method, more preferably a hot-melt extrusion method from which the amorphous sofosbuvir is obtained, or is dissolved in at least one solvent, and the obtained solution is subjected to at least one treatment stage from which the amorphous sofosbuvir is obtained. Preferably, the crystalline and/or amorphous sofosbuvir, preferably the crystalline sofosbuvir, is dissolved in at least one solvent, and the obtained solution is subjected to at least one treatment stage from which the amorphous sofosbuvir is obtained. Regarding the at least one solvent, no specific restrictions exist. Preferably, the at least one solvent is selected from the group consisting of water, C1-C3 ketones, C1-C2 halogenated hydrocarbons, C1-C4 alcohols, C2-C6 ethers, C3-C5 esters, and a combination of two or more thereof, more preferably from the group consisting of water, C1-C4 alcohols, C1-C3 ketones, and a combination of two or more thereof, wherein more preferably, the at least one solvent comprises, more preferably consists of, water and C1-C4 alcohol, preferably water and ethanol, or comprises, more preferably consists of, acetone. Regarding the at least one treatment stage from which the amorphous sofosbuvir is obtained, no specific restrictions exist, provided that the amorphous sofosbuvir is obtained. Preferably, the treatment stage comprises subjecting at least a portion of the solution of the sofosbuvir to lyophilization or rapid-drying, preferably to rapid-drying, wherein the rapid-drying preferably comprises at least one atomization process, and is more preferably carried out by spray-drying or spray-granulation, preferably by spray-drying. Prior to the rapid-drying, the solution of the sofosbuvir can be concentrated with respect to the sofosbuvir content, for example by filtration, centrifugation, evaporation, adding sofosbuvir to the solution, or a combination of two or more of these methods. The preferred rapid-drying method, the spray-drying, is not subjected to specific restrictions provided that the amorphous sofosbuvir is obtained. For example, the inlet temperature used may be in the range of from 50 to 100° C. For example, the outlet temperature used may be in the range of from 20 to 70° C.

Based on said solid composition which is most preferably a solid dispersion, it is especially preferred that directly after (ii), the solid composition is processed to the pharmaceutical composition which, as described above, comprises, in addition to the solid composition, preferably at least one pharmaceutically acceptable excipient. Therefore, (ii) preferably comprises preparing a mixture of the solid composition obtained from (i) and at least one pharmaceutically acceptable excipient. With regard to the generally suitable and preferred pharmaceutically acceptable excipients and their respective preferred amounts, reference is made to the respective discussion above. In particular, the solid composition obtained from (i) and the at least one pharmaceutically acceptable excipient are employed according to (ii) in amounts so that from 30 to 65 weight-%, preferably from 35 to 55 weight-%, more preferably from 40 to 45 weight-%, more preferably from 40 to 42 weight-% of the solid pharmaceutical composition obtained from (ii) consist of the solid composition.

Preferably, according to (ii), the mixture is processed to a tablet. Therefore, no specific restrictions exist. For example, for tableting purposes, the mixture can be subjected to an absolute pressure in the range of from 100 to 300 bar, preferably in the range of from 150 to 250 bar using a suitable tableting apparatus.

Optionally, the respectively obtained tablet obtained can be coated with at least one coating excipient described hereinabove.

The solid pharmaceutical composition, preferably the oral dosage form, more preferably the tablet of the present invention is preferably used in a method for treating hepatitis C in a human. Therefore, the present invention also relates to a solid pharmaceutical composition as described above, for use in a method for treating hepatitis C in a human. Further, the present invention relates to the use of a solid pharmaceutical composition as described above for treating hepatitis C in a human. Further, the present invention relates to the use of a solid pharmaceutical composition as described above for the preparation of a medicament for treating hepatitis C in a human. Further, the present invention relates to a method for treating hepatitis C comprising administering a solid pharmaceutical composition as described above to a human patient in need thereof.

Generally, the present invention relates to the use of a solid composition comprising sofosbuvir according to formula (I)

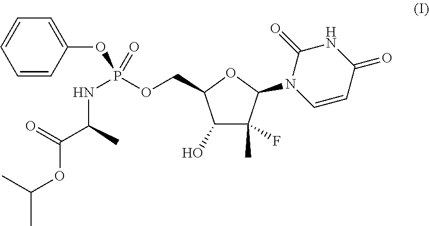 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

and at least one pharmaceutically acceptable matrix compound, wherein at least 99 weight-% of the sofosbuvir comprised in the solid composition are present in amorphous form, at least 99 weight-% of the solid composition consist of the sofosbuvir and the at least one matrix compound, wherein the solid composition contains the sofosbuvir in an amount of at least 25 weight-%, or at least 30 weight-%, or at least 35 weight-%, or at least 40 weight-% or at least 50 weight-% or preferably at least 55 weight-% based on the combined weight of the sofosbuvir and the at least one pharmaceutically acceptable matrix compound, wherein in the adsorption-desorption isotherm of the at least one pharmaceutically acceptable matrix compound, ΔΔm/% which is defined as the mass difference Δm(desorption)/% at 75% relative humidity and 25° C. minus the mass difference Δm(adsorption)/% at 75% relative humidity and 25° C., determined according to dynamic vapor sorption measurement, in particular determined according to dynamic vapor sorption measurement as described in Reference Example 1 herein, is <0, for preparing a solid pharmaceutical composition having a moisture stability of at least 95%, wherein the moisture stability is defined as the amount of solid amorphous sofosbuvir which is present in the solid pharmaceutical composition after having been exposed to a relative humidity of 75% at 40° C. for 8 weeks, relative to the amount of solid amorphous sofosbuvir which is present in the solid pharmaceutical composition before said exposure, said moisture stability preferably being determined as described in Reference Example 2 herein, wherein the solid composition, preferably being a tablet, is preferably used in combination with at least one pharmaceutically acceptable excipient preferably comprising, more preferably being, a combination of mannitol, microcrystalline cellulose, croscarmellose sodium, colloidal silica, magnesium stearate.

The present invention is illustrated by the following embodiments and combinations of embodiments resulting from the given dependencies and back-references:
1. A solid pharmaceutical composition comprising a solid composition, wherein the solid composition comprises sofosbuvir according to formula (I)

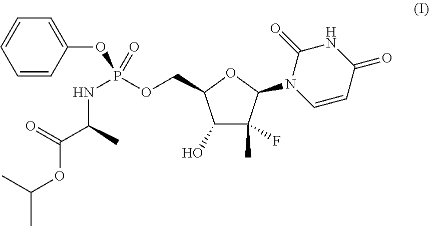 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

and at least one pharmaceutically acceptable matrix compound, wherein at least 99 weight-% of the sofosbuvir comprised in the solid composition are present in amorphous form, at least 99 weight-% of the solid composition consist of the sofosbuvir and the at least one matrix compound, wherein the solid composition contains the sofosbuvir in an amount of at least 25 weight-%, or at least 30 weight-%, or at least 35 weight-%, or at least 40 weight-% or at least 50 weight-% or preferably at least 55 weight-% based on the combined weight of the sofosbuvir and the at least one matrix compound, wherein in the adsorption-desorption isotherm of the at least one pharmaceutically acceptable matrix compound, ΔΔm/% which is defined as the mass difference Δm(desorption)/% at 75% relative humidity and 25° C. minus the mass difference Δm(adsorption)/% at 75% relative humidity and 25° C., determined according to dynamic vapor sorption measurement, in particular determined according to dynamic vapor sorption measurement as described in Reference Example 1 herein, is <0.
2. The solid pharmaceutical composition of embodiment 1, wherein the solid composition comprises the sofosbuvir in an amount of at least 55 weight % or preferably in the range of from 55 to 95 weight-%, preferably from 65 to 90 weight-%, more preferably from 70 to 85 weight-% or from 70 to 75 weight-% or from 75 to 80 weight-% or from 80 to 85 weight-%, based on the combined weight of the sofosbuvir and the at least one matrix compound.
3. The solid pharmaceutical composition of embodiment 1 or 2, wherein ΔΔm/% is in the range of from −1.0≤ΔΔm/%<0, preferably in the range of from −0.8≤ΔΔm/%<0, more preferably in the range of from −0.6≤ΔΔm/%<0.
4. The solid pharmaceutical composition of any one of embodiments 1 to 3, wherein ΔΔm/% is in the range of from −0.1≤ΔΔm/%<0.
5. The solid pharmaceutical composition of any one of embodiments 1 to 4, wherein the at least one matrix compound is selected from the group consisting of hydrophilic polymers, silicon-based inorganic adsorbents and a combination of two or more thereof.
6. The solid pharmaceutical composition of embodiment 5, wherein the hydrophilic polymers include, preferably are, one or more of polysaccharides, preferably cellulose derivatives, polyvinylpyrrolidones, polyethylene glycols, polyethylene glycol based copolymers, polyacrylic acids, salts of polyacrylic acids, polyvinyl alcohols, polyacrylamide copolymers, methacrylic acid copolymers, methacrylate copolymers, pectines, chitin derivatives, chitosan derivatives, polyphosphates, polyoxazolines.
7. The solid pharmaceutical composition of embodiment 5 or 6, wherein the silicon-based inorganic adsorbents include, preferably are, one or more of silica and silicates.
8. The solid pharmaceutical composition of any one of embodiments 1 to 6, wherein the at least one matrix compound is selected from the group consisting of hydrophilic water-soluble polymers and a combination of two or more thereof.
9. The solid pharmaceutical composition of embodiment 8, wherein the at least one hydrophilic water-soluble polymer has a solubility in water of at least 10 g/l, preferably of at least 20 g/l, more preferably of at least 30 g/l, in each case at 23° C. at atmospheric pressure.
10. The solid pharmaceutical composition of embodiment 8 or 9, wherein the at least one hydrophilic water-soluble polymer comprises, preferably consists of, at least one vinyl pyrrolidone-vinyl acetate copolymer.
11. The solid pharmaceutical composition of any one of embodiments 8 to 10, wherein the at least one hydrophilic water-soluble polymer comprises, preferably consists of, copovidone.
12. The solid pharmaceutical composition of any one of embodiments 8 to 11, wherein the weight average molecular weight (M_{w}) of the at least one hydrophilic water-soluble polymer is in the range of from 20 to 100 kDa, preferably in the range of from 30 to 85 kDa, more preferably in the range of from 40 to 75 kDa.
13. The solid pharmaceutical composition of any one of embodiments 1 to 12, wherein at least 99.5 weight-%, preferably at least 99.9 weight-% of the solid composition comprised in pharmaceutical composition consist of the sofosbuvir and the at least one matrix compound.
14. The solid pharmaceutical composition of any one of embodiments 1 to 13, wherein the solid composition comprised in pharmaceutical composition comprises less than 0.1 weight-%, preferably less than 0.01 weight-%, more preferably less than 0.001 weight-% of a surfactant.
15. The solid pharmaceutical composition of any one of embodiments 1 to 14, wherein the solid composition is a solid dispersion.
16. The solid pharmaceutical composition of any one of embodiments 1 to 15, being an oral dosage form, preferably a tablet.
17. The solid pharmaceutical composition of any one of embodiments 1 to 16, further comprising, in addition to the sofosbuvir, one or more further anti-HCV agents preferably the further anti-HCV agents being ledipasvir according to formula (II)

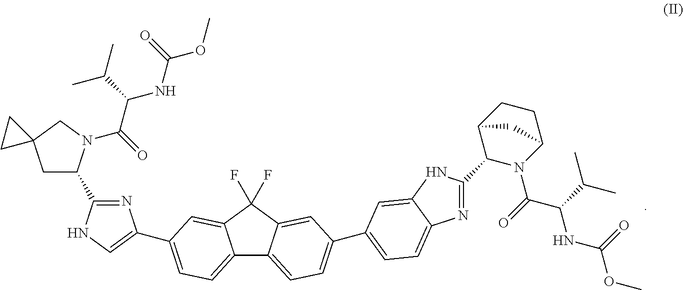 [SMILES: COC(=O)N[C@H](C(=O)N1CC2(CC2)C[C@H]1c1nc(-c2ccc3c(c2)C(F)(F)c2cc(-c4ccc5nc([C@@H]6[C@H]7CC[C@H](C7)N6C(=O)[C@@H](NC(=O)OC)C(C)C)[nH]c5c4)ccc2-3)c[nH]1)C(C)C]

18. The solid pharmaceutical composition of any one of embodiments 1 to 17, comprising the solid composition and at least one pharmaceutically acceptable excipient, wherein more preferably, the solid pharmaceutical composition consists of the solid composition, the at least one pharmaceutically acceptable excipient and optionally the one or more further anti-HCV agents.
19. The solid pharmaceutical composition of embodiment 18, wherein at least one of the at least one pharmaceutically acceptable excipient is different from the at least one pharmaceutically acceptable matrix compound comprised in the solid composition.
20. The solid pharmaceutical composition of embodiment 18 or 19, wherein the at least one pharmaceutically acceptable excipient is different from the at least one pharmaceutically acceptable matrix compound comprised in the solid composition.
21. The solid pharmaceutical composition of any one of embodiments 18 to 20, wherein the at least one pharmaceutically acceptable excipient is not a pharmaceutically acceptable matrix compound as defined in embodiment 10 or 11.
22. The solid pharmaceutical composition of any one of embodiments 18 to 21, wherein the at least one pharmaceutically acceptable excipient includes one or more of at least one of a diluent, at least one disintegrant, at least one glidant, at least one lubricant, and combinations of two or more thereof.
23. The solid pharmaceutical composition of embodiment 22, wherein the at least one diluent includes one or more of calcium carbonate, dicalcium phosphate, dry starch, calcium sulfate, cellulose, compressible sugars, confectioner's sugar, dextrates, dextrin, dextrose, dibasic calcium phosphate dihydrate, glyceryl palmitostearate, hydrogenated vegetable oil, inositol, kaolin, lactose, magnesium carbonate, magnesium oxide, maltodextrin, mannitol, microcrystalline cellulose, polymethacrylates, potassium chloride, powdered cellulose, powdered sugar, pregelatinized starch, sodium chloride, sorbitol, starch, sucrose, sugar spheres, talc, tribasic calcium phosphate.
24. The solid pharmaceutical composition of embodiment 22 or 23, wherein the at least one disintegrant includes one or more of agar, alginic acid, bentonite, carboxymethylcellulose calcium, carboxymethylcellulose sodium, carboxymethylcellulose, cellulose, a cation exchange resin, cellulose, gums, citrus pulp, colloidal silicon dioxide, corn starch, croscarmellose sodium, crospovidone, guar gum, hydrous aluminum silicate, an ion exchange resin such as polyacrin potassium, magnesium aluminum silicate, methyl cellulose, microcrystalline cellulose, modified cellulose gum, modified corn starch, montmorillonite clay, natural sponge, polyacrilin potassium, potato starch, powdered cellulose, povidone, pregelatinized starch, sodium alginate, sodium bicarbonate optionally in admixture with one or more acidulants, sodium starch glycolate, starch, silicates.
25. The solid pharmaceutical composition of any one of embodiments 22 to 24, wherein the at least one glidant includes one or more of colloidal silicon dioxide, talc, starch, starch derivatives.
26. The solid pharmaceutical composition of any one of embodiments 22 to 25, wherein the at least one lubricant includes one or more of calcium stearate, glyceryl monostearate, glyceryl palmitostearate, hydrogenated castor oil, hydrogenated vegetable oil, light mineral oil, magnesium stearate, mineral oil, polyethylene glycol, sodium benzoate, sodium lauryl sulfate, sodium stearyl fumarate, stearic acid, talc, zinc stearate.
27. The solid pharmaceutical composition of any one of embodiments 22 to 26, wherein the at least one pharmaceutically acceptable excipient comprises, preferably is, a combination of at least one a diluent, at least one disintegrant, at least one glidant, and at least one lubricant.
28. The solid pharmaceutical composition of any one of embodiments 18 to 27, wherein from 30 to 65, preferably from 35 to 55, more preferably from 40 to 45 weight-% of the solid pharmaceutical composition consist of the solid composition.
29. The solid pharmaceutical composition of any one of embodiments 18 to 28, wherein from 40 to 42 weight-% of the solid pharmaceutical composition consist of the solid composition.
30. The solid pharmaceutical composition of any one of embodiments 18 to 29, wherein the at least one pharmaceutically acceptable excipient comprises, preferably is, a combination of mannitol, microcrystalline cellulose, croscarmellose sodium, colloidal silica, magnesium stearate.
31. The solid pharmaceutical composition of embodiment 30, comprising from 15 to 25 weight-%, preferably from 20 to 22 weight-% mannitol, from 25 to 35 weight-%, preferably from 30 to 32 weight-% microcrystalline cellulose, from 2 to 10 weight-%, preferably from 4 to 6 weight-% croscarmellose sodium, from 0.2 to 2 weight-%, preferably from 0.5 to 1.5 weight-% colloidal silica, from 0.5 to 5 weight-%, preferably from 1 to 1.5 weight-% magnesium stearate, in each based on the total weight of the solid pharmaceutical composition.
32. The solid pharmaceutical composition of any one of embodiments 18 to 31, comprising from 40 to 41 weight-% of the solid composition, from 20 to 21 weight-% mannitol, from 30 to 31 weight-% microcrystalline cellulose, from 4.5 to 5.5 weight-% croscarmellose sodium, from 0.5 to 1.0 weight-% colloidal silica, from 1.0 to 2.0 weight-% magnesium stearate, in each based on the total weight of the solid pharmaceutical composition, wherein the individual contents add up to 100%.
33. The solid pharmaceutical composition of any one of embodiments 1 to 32, wherein the solid composition is prepared by a process comprising embedding sofosbuvir in a matrix comprising, preferably consisting of the at least one pharmaceutically acceptable matrix compound, wherein the weight ratio of the sofosbuvir relative to the at least one matrix compound is at least 5.5:4.5, preferably in the range of from 5.5:4.5 to 9.5:0.5, more preferably in the range of from 6.5:3.5 to 9.0:1.0, more preferably in the range of from 7.5:2.5 to 8.5:1.5
34. The solid pharmaceutical composition of embodiment 33, wherein the solid composition is prepared by a process comprising embedding sofosbuvir in a matrix comprising, preferably consisting of the at least one pharmaceutically acceptable matrix compound, wherein said embedding comprises melting the at least one pharmaceutically acceptable matrix compound together with the sofosbuvir, or wherein said embedding comprises preparing a solution of the sofosbuvir and the at least one pharmaceutically acceptable matrix compound in at least one solvent preferably selected from the group consisting of water, an organic solvent, and a combination of two or more thereof, more preferably selected from the group consisting of a C1-C2 halogenated hydrocarbon, a C1-C4 alcohol, a C3-C6 ketone, a C2-C6 ether, a C3-C5 ester, and a combination of two or more thereof, wherein said solution is preferably subjected to drying, preferably by lyophilizing the solution or spray-drying the solution.
35. The solid pharmaceutical composition of embodiment 33 or 34, wherein the solid composition is prepared by a process comprising embedding sofosbuvir in a matrix comprising, preferably consisting of the at least one pharmaceutically acceptable matrix compound, by melting the at least one pharmaceutically acceptable matrix compound in solid form together with the sofosbuvir in solid form, preferably by a hot-melt method, more preferably by a hot-melt extrusion method.
36. The solid pharmaceutical composition of embodiment 35, wherein the sofosbuvir in solid form is sofosbuvir in at least one crystalline form or in amorphous form or as a mixture of at least one crystalline form and amorphous form, wherein preferably at least 95 weight-%, preferably at least 99 weight-%, more preferably at least 99.9 weight-% of the sofosbuvir are present in at least one crystalline form.
37. The solid pharmaceutical composition of any one of embodiments 33 to 36, wherein at least 99 weight-%, preferably at least 99.5 weight-%, more preferably at least 99.9 weight-% of the sofosbuvir comprised in the solid composition are present in amorphous form directly after preparing the solid composition.
38. The solid pharmaceutical composition of any one of embodiments 1 to 37, obtainable or obtained by a process comprising
   (i) preparing a solid composition comprising sofosbuvir and at least one pharmaceutically acceptable matrix compound, wherein at least 99 weight-% of the sofosbuvir comprised in the solid composition are present in amorphous form, at least 99 weight-% of the solid composition consist of the sofosbuvir and the at least one matrix compound, wherein the solid composition contains the sofosbuvir in an amount of at least 25 weight-%, or at least 30 weight-%, or at least 35 weight-%, or at least 40 weight-% or at least 50 weight-% or preferably at least 55 weight-% based on the combined weight of the sofosbuvir and the at least one matrix compound,
   (ii) processing the solid composition obtained from (i) to the pharmaceutical composition.
39. The solid pharmaceutical composition of embodiment 38, said process comprising
   (ii) directly processing the solid composition obtained from (i) to the pharmaceutical composition.
40. The solid pharmaceutical composition of embodiment 38 or 39, wherein after (i) and before (ii), the solid composition obtained from (i) is not subjected to any modification.
41. The solid pharmaceutical composition of any one of embodiments 38 to 40, wherein the solid composition obtained from (i) is processed to the pharmaceutical composition according to (ii) at most 168 h, preferably at most 72 h, more preferably at most 24 h after having been obtained from (i), wherein during this period of time, the solid composition is preferably not subjected to stress conditions of 40° C. and a relative humidity of 75%, more preferably stored under ambient conditions.
42. The solid pharmaceutical composition of any one of embodiments 1 to 41, having a moisture stability of at least 95%, preferably at least 98%, more preferably at least 99%, wherein the moisture stability is defined as the amount of solid amorphous sofosbuvir which is present in the solid pharmaceutical composition after having been exposed to a relative humidity of 75% at 40° C. for 8 weeks, relative to the amount of solid amorphous sofosbuvir which is present in the solid pharmaceutical composition before said exposure, said moisture stability preferably being determined as described in Reference Example 2 herein.
43. The solid composition of any one of embodiments 1 to 42 for use in a method for treating hepatitis C in a human.
44. The solid composition of any one of embodiments 1 to 43 for treating hepatitis C in a human.
45. A process for preparing a solid pharmaceutical composition according to any one of embodiments 1 to 44, comprising
   (i) preparing a solid composition comprising sofosbuvir and at least one pharmaceutically acceptable matrix compound, wherein at least 99 weight-% of the sofosbuvir comprised in the solid composition are present in amorphous form, at least 99 weight-% of the solid composition consist of the sofosbuvir and the at least one matrix compound, wherein the solid composition contains the sofosbuvir in an amount of at least 25 weight-%, or at least 30 weight-%, or at least 35 weight-%, or at least 40 weight-% or at least 50 weight-% or preferably at least 55 weight-% based on the combined weight of the sofosbuvir and the at least one matrix compound,
   (ii) processing the solid composition obtained from (i) to the pharmaceutical composition;
   wherein in the adsorption-desorption isotherm of the at least one pharmaceutically acceptable matrix compound, ΔΔm/% which is defined as the mass difference Δm(desorption)/% at 75% relative humidity and 25° C. minus the mass difference Δm(adsorption)/% at 75% relative humidity and 25° C., determined according to dynamic vapor sorption measurement, in particular determined according to dynamic vapor sorption measurement as described in Reference Example 1 herein, is <0.
46. The process of embodiment 45, wherein ΔΔm/% is in the range of from −1.0≤ΔΔm/%<0, preferably in the range of from −0.8≤ΔΔm/%<0, more preferably in the range of from −0.6≤ΔΔm/%<0.
47. The process of embodiment 45 or 46, wherein ΔΔm/% is in the range of from −0.1≤ΔΔm/%<0.
48. The process of any one of embodiments 45 to 47, comprising
   (ii) directly processing the solid composition obtained from (i) to the pharmaceutical composition.
49. The process of any one of embodiments 45 to 48, wherein after (i) and before (ii), the solid composition obtained from (i) is not subjected to any modification.
50. The process of any one of embodiments 45 to 49, wherein the solid composition obtained from (i) is processed to the pharmaceutical composition according to (ii) at most 168 h, preferably at most 72 h, more preferably at most 24 h after having been obtained from (i), wherein during this period of time, the solid composition is preferably not subjected to stress conditions of 40° C. and a relative humidity of 75%, more preferably stored under ambient conditions.
51. The process of any one of embodiments 45 to 50, wherein the solid composition is a solid dispersion.
52. The process of any one of embodiments 45 to 51, wherein (i) comprises embedding the sofosbuvir in a matrix comprising, preferably consisting of the at least one pharmaceutically acceptable matrix compound, wherein the weight ratio of the sofosbuvir relative to the at least one matrix compound is at least 5.5:4.5, preferably in the range of from 5.5:4.5 to 9.5:0.5, more preferably in the range of from 6.5:3.5 to 9.0:1.0, more preferably in the range of from 7.5:2.5 to 8.5:1.5.
53. The process of embodiment 52, wherein (i) comprises embedding the sofosbuvir in a matrix comprising, preferably consisting of the at least one pharmaceutically acceptable matrix compound, wherein said embedding comprises melting the at least one pharmaceutically acceptable matrix compound together with the sofosbuvir, or wherein said embedding comprises preparing a solution of the sofosbuvir in at least one solvent preferably selected from the group consisting of water, an organic solvent, and a combination of two or more thereof, more preferably selected from the group consisting of a C1-C2 halogenated hydrocarbon, a C1-C4 alcohol, a C3-C6 ketone, a C2-C6 ether, a C3-C5 ester, and a combination of two or more thereof, wherein said solution is preferably subjected to drying, preferably by lyophilizing the solution or spray-drying the solution.
54. The process of embodiment 52 or 53, wherein (i) comprises embedding sofosbuvir in a matrix comprising, preferably consisting of the at least one pharmaceutically acceptable matrix compound, by melting the at least one pharmaceutically acceptable matrix compound in solid form together with the sofosbuvir in solid form, preferably by a hot-melt method, more preferably by a hot-melt extrusion method.
55. The process of embodiment 54, wherein (i) comprises melting at a temperature in the range of from 75 to 175° C., preferably in the range of from 90 to 150° C.
56. The process of embodiment 54 or 55, wherein after the melting according to (i), (i) comprises cooling, preferably to a temperature in the range of from 10 to 40° C., preferably in the range of from 20 to 30° C.
57. The process of any one of embodiments 54 to 56, wherein the sofosbuvir in solid form is sofosbuvir in at least one crystalline form or in amorphous form or as a mixture of at least one crystalline form and amorphous form, wherein preferably at least 95 weight-%, preferably at least 99 weight-%, more preferably at least 99.9 weight-% of the sofosbuvir are present in at least one crystalline form.
58. The process of any one of embodiments 45 to 57, wherein at least 99 weight-%, preferably at least 99.5 weight-%, more preferably at least 99.9 weight-% of the sofosbuvir comprised in the solid composition are present in amorphous form directly after preparing the solid composition.
59. The process of any one of embodiments 45 to 58, wherein (ii) comprises preparing a mixture of the solid composition obtained from (i) and at least one pharmaceutically acceptable excipient.
60. The process of embodiment 59, wherein at least one of the at least one pharmaceutically acceptable excipient is different from the at least one pharmaceutically acceptable matrix compound comprised in the solid composition, preferably wherein the at least one pharmaceutically acceptable excipient is different from the at least one pharmaceutically acceptable matrix compound comprised in the solid composition, more preferably wherein the at least one pharmaceutically acceptable excipient is not a pharmaceutically acceptable matrix compound as defined in embodiment 10 or 11.
61. The process of embodiment 59 or 60, wherein the at least one pharmaceutically acceptable excipient includes one or more of at least one of a diluent, at least one disintegrant, at least one glidant, at least one lubricant, and a combination of two or more thereof.
62. The process of embodiment 61, wherein the at least one diluent includes one or more of calcium carbonate, dicalcium phosphate, dry starch, calcium sulfate, cellulose, compressible sugars, confectioner's sugar, dextrates, dextrin, dextrose, dibasic calcium phosphate dihydrate, glyceryl palmitostearate, hydrogenated vegetable oil, inositol, kaolin, lactose, magnesium carbonate, magnesium oxide, maltodextrin, mannitol, microcrystalline cellulose, polymethacrylates, potassium chloride, powdered cellulose, powdered sugar, pregelatinized starch, sodium chloride, sorbitol, starch, sucrose, sugar spheres, talc, tribasic calcium phosphate, wherein the at least one disintegrant includes one or more of agar, alginic acid, bentonite, carboxymethylcellulose calcium, carboxymethylcellulose sodium, carboxymethylcellulose, cellulose, a cation exchange resin, cellulose, gums, citrus pulp, colloidal silicon dioxide, corn starch, croscarmellose sodium, crospovidone, guar gum, hydrous aluminum silicate, an ion exchange resin such as polyacrin potassium, magnesium aluminum silicate, methyl cellulose, microcrystalline cellulose, modified cellulose gum, modified corn starch, montmorillonite clay, natural sponge, polyacrilin potassium, potato starch, powdered cellulose, povidone, pregelatinized starch, sodium alginate, sodium bicarbonate optionally in admixture with one or more acidulants, sodium starch glycolate, starch, silicates, wherein the at least one glidant includes one or more of colloidal silicon dioxide, talc, starch, starch derivatives, wherein the at least one lubricant includes one or more of calcium stearate, glyceryl monostearate, glyceryl palmitostearate, hydrogenated castor oil, hydrogenated vegetable oil, light mineral oil, magnesium stearate, mineral oil, polyethylene glycol, sodium benzoate, sodium lauryl sulfate, sodium stearyl fumarate, stearic acid, talc, zinc stearate, wherein the at least one pharmaceutically acceptable excipient preferably comprises, more preferably is, a combination of at least one a diluent, at least one disintegrant, at least one glidant, and at least one lubricant.
63. The process of any one of embodiments 59 to 62, wherein the solid composition obtained from (i) and the at least one pharmaceutically acceptable excipient are employed in amounts so that from 30 to 65, preferably from 35 to 55, more preferably from 40 to 45 weight-% of the solid pharmaceutical composition obtained from (ii) consist of the solid composition.
64. The process of embodiment 63, wherein from 40 to 42 weight-% of the solid pharmaceutical composition consist of the solid composition.
65. The process of any one of embodiments 59 to 64, wherein the at least one pharmaceutically acceptable excipient comprises, preferably is, a combination of mannitol, microcrystalline cellulose, croscarmellose sodium, colloidal silica, magnesium stearate.
66. The process of any one of embodiments 59 to 65, wherein (ii) comprises processing the mixture to a tablet.
67. The process of embodiment 66, wherein processing the mixture to a tablet according to (ii) comprises subjecting the mixture to an absolute pressure in the range of from 100 to 300 bar, preferably in the range of from 150 to 250 bar.
68. Use of a solid pharmaceutical composition according to any one of embodiments 1 to 42 for the preparation of a medicament for treating hepatitis C in a human.
69. A method for treating hepatitis C comprising administering a solid pharmaceutical composition according to any one of embodiments 1 to 42 to a human in need thereof.
70. Use of a solid composition comprising sofosbuvir according to formula (I)

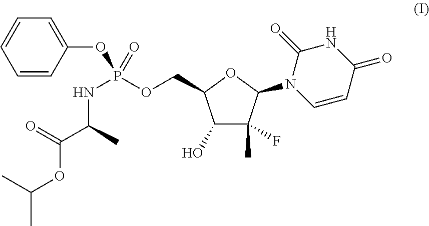 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

and at least one pharmaceutically acceptable matrix compound, wherein at least 99 weight-% of the sofosbuvir comprised in the solid composition are present in amorphous form, at least 99 weight-% of the solid composition consist of the sofosbuvir and the at least one matrix compound, wherein the solid composition contains the sofosbuvir in an amount of at least 25 weight-%, or at least 30 weight-%, or at least 35 weight-%, or at least 40 weight-% or at least 50 weight-% or preferably at least 55 weight-% based on the combined weight of the sofosbuvir and the at least one pharmaceutically acceptable matrix compound, wherein in the adsorption-desorption isotherm of the at least one pharmaceutically acceptable matrix compound, ΔΔm/% which is defined as the mass difference Δm(desorption)/% at 75% relative humidity and 25° C. minus the mass difference Δm(adsorption)/% at 75% relative humidity and 25° C., determined according to dynamic vapor sorption measurement, in particular determined according to dynamic vapor sorption measurement as described in Reference Example 1 herein, is <0, for preparing a solid pharmaceutical composition having a moisture stability of at least 95%, wherein the moisture stability is defined as the amount of solid amorphous sofosbuvir which is present in the solid pharmaceutical composition after having been exposed to a relative humidity of 75% at 40° C. for 8 weeks, relative to the amount of solid amorphous sofosbuvir which is present in the solid pharmaceutical composition before said exposure, said moisture stability preferably being determined as described in Reference Example 2 herein, wherein preferably the solid composition is according to any one of embodiments 1 to 42.
71. The use of embodiment 70, wherein ΔΔm/% is in the range of from −1.0≤ΔΔm/%<0, preferably in the range of from −0.8≤ΔΔm/%<0, more preferably in the range of from −0.6≤ΔΔm/%<0, more preferably in the range of from −0.1≤ΔΔm/%<0.
72. The use of embodiment 70 or 71, wherein the solid composition comprises the sofosbuvir in an amount in the range of from 55 to 95 weight-%, preferably from 65 to 90 weight-%, more preferably from 75 to 85 weight-%, based on the combined weight of the sofosbuvir and the at least one matrix compound.
73. The use of any one of embodiments 70 to 72, wherein the solid pharmaceutical composition is an oral dosage form, preferably a tablet.
74. The use of any one of embodiments 70 to 73, wherein the solid pharmaceutical composition has a moisture stability of at least 98%, preferably at least 99%, said moisture stability preferably being determined as described in Reference Example 2 herein.
75. The use of any one of embodiments 70 to 74, wherein the solid composition is a solid dispersion.
76. The use of any one of embodiments 70 to 75, wherein the solid composition is used in combination with at least one pharmaceutically acceptable excipient preferably comprising, more preferably being, a combination of mannitol, microcrystalline cellulose, croscarmellose sodium, colloidal silica, magnesium stearate.

According to a further aspect, the present invention is illustrated by the following embodiments and combinations of embodiments resulting from the given dependencies and back-references:
I. A solid pharmaceutical composition comprising a solid composition, wherein the solid composition comprises sofosbuvir according to formula (I)

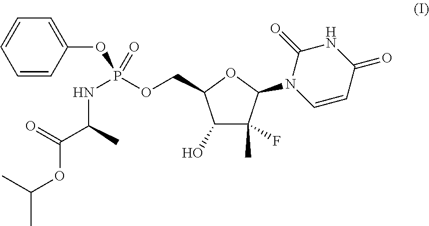 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

and at least one pharmaceutically acceptable matrix compound, wherein at least 99 weight-% of the sofosbuvir comprised in the solid composition are present in amorphous form, at least 99 weight-% of the solid composition consist of the sofosbuvir and the at least one matrix compound, wherein the solid composition contains the sofosbuvir in an amount of at least 25 weight-%, or at least 30 weight-%, or at least 35 weight-%, or at least 40 weight-% or at least 50 weight-% or preferably at least 55 weight-% based on the combined weight of the sofosbuvir and the at least one matrix compound,
wherein the at least one pharmaceutically acceptable matrix compound comprises, preferably consists of, at least one vinyl pyrrolidone-vinyl acetate copolymer.
II. The solid pharmaceutical composition of embodiment I, wherein the at least one pharmaceutically acceptable matrix compound comprises, preferably consists of, copovidone.
III. The solid pharmaceutical composition of any one of embodiments I to III, wherein the weight average molecular weight (M_{w}) of the at least one pharmaceutically acceptable matrix compound is in the range of from 20 to 100 kDa, preferably in the range of from 30 to 85 kDa, more preferably in the range of from 40 to 75 kDa.
IV. The solid pharmaceutical composition of any one of embodiments I to III, wherein at least 99.5 weight-%, preferably at least 99.9 weight-% of the solid composition comprised in pharmaceutical composition consist of the sofosbuvir and the at least one matrix compound.
V. The solid pharmaceutical composition of any one of embodiments I to IV, wherein the solid composition comprised in pharmaceutical composition comprises less than 0.1 weight-%, preferably less than 0.01 weight-%, more preferably less than 0.001 weight-% of a surfactant.
VI. The solid pharmaceutical composition of any one of embodiments I to IV, wherein in the adsorption-desorption isotherm of the at least one pharmaceutically acceptable matrix compound, ΔΔm/% which is defined as the mass difference Δm(desorption)/% at 75% relative humidity and 25° C. minus the mass difference Δm(adsorption)/% at 75% relative humidity and 25° C., determined according to dynamic vapor sorption measurement, in particular determined according to dynamic vapor sorption measurement as described in Reference Example 1 herein, is in the range of from −0.1≤ΔΔm/%<0, preferably in the range of from −0.1≤ΔΔm/%≤0.001.
VII. The solid pharmaceutical composition of any one embodiments I to VI, wherein the solid composition comprises the sofosbuvir in an amount of at least 25 weight-%, or at least 30 weight-%, or at least 35 weight-%, or at least 40 weight-% or at least 50 weight-% or preferably of at least 55 weight-% or preferably in the range of from 55 to 95 weight-%, preferably from 65 to 90 weight-%, more preferably from 75 to 85 weight-%, based on the combined weight of the sofosbuvir and the at least one matrix compound.
VIII. The solid pharmaceutical composition of any one of embodiments I to VII, wherein the solid composition is a solid dispersion.
IX. The solid pharmaceutical composition of any one of embodiments I to VIII, being an oral dosage form, preferably a tablet.
X. The solid pharmaceutical composition of any one of embodiments I to XI, comprising the solid composition and at least one pharmaceutically acceptable excipient, wherein preferably, the solid pharmaceutical composition consists of the solid composition and the at least one pharmaceutically acceptable excipient, and the at least one pharmaceutically acceptable excipient is different from the at least one pharmaceutically acceptable matrix compound comprised in the solid composition.
XI. The solid pharmaceutical composition of embodiment X, wherein the at least one pharmaceutically acceptable excipient includes one or more of at least one of a diluent, at least one disintegrant, at least one glidant, at least one lubricant, and combinations of two or more thereof, preferably a combination of at least one of a diluent, at least one disintegrant, at least one glidant, at least one lubricant, more preferably a combination of a diluent, a disintegrant, a glidant, and a lubricant.
XII. The solid pharmaceutical composition of embodiment XI,
   wherein the at least one diluent includes one or more of calcium carbonate, dicalcium phosphate, dry starch, calcium sulfate, cellulose, compressible sugars, confectioner's sugar, dextrates, dextrin, dextrose, dibasic calcium phosphate dihydrate, glyceryl palmitostearate, hydrogenated vegetable oil, inositol, kaolin, lactose, magnesium carbonate, magnesium oxide, maltodextrin, mannitol, microcrystalline cellulose, polymethacrylates, potassium chloride, powdered cellulose, powdered sugar, pregelatinized starch, sodium chloride, sorbitol, starch, sucrose, sugar spheres, talc, tribasic calcium phosphate;
   wherein the at least one disintegrant includes one or more of agar, alginic acid, bentonite, carboxymethylcellulose calcium, carboxymethylcellulose sodium, carboxymethylcellulose, cellulose, a cation exchange resin, cellulose, gums, citrus pulp, colloidal silicon dioxide, corn starch, croscarmellose sodium, crospovidone, guar gum, hydrous aluminum silicate, an ion exchange resin such as polyacrin potassium, magnesium aluminum silicate, methyl cellulose, microcrystalline cellulose, modified cellulose gum, modified corn starch, montmorillonite clay, natural sponge, polyacrilin potassium, potato starch, powdered cellulose, povidone, pregelatinized starch, sodium alginate, sodium bicarbonate optionally in admixture with one or more acidulants, sodium starch glycolate, starch, silicates;
   wherein the at least one glidant includes one or more of colloidal silicon dioxide, talc, starch, starch derivatives; and
   wherein the at least one lubricant includes one or more of calcium stearate, glyceryl monostearate, glyceryl palmitostearate, hydrogenated castor oil, hydrogenated vegetable oil, light mineral oil, magnesium stearate, mineral oil, polyethylene glycol, sodium benzoate, sodium lauryl sulfate, sodium stearyl fumarate, stearic acid, talc, zinc stearate.
XIII. The solid pharmaceutical composition of any one of embodiments X to XII, wherein from 30 to 65 weight-%, preferably from 35 to 55 weight-%, more preferably from 40 to 45 weight-%, more preferably from 40 to 42 weight-% of the solid pharmaceutical composition consist of the solid composition.
XIV. The solid pharmaceutical composition of any one of embodiments X to XIII, wherein the at least one pharmaceutically acceptable excipient comprises, preferably is, a combination of mannitol, microcrystalline cellulose, croscarmellose sodium, colloidal silica, magnesium stearate, said solid pharmaceutical composition preferably comprising from 15 to 25 weight-%, preferably from 20 to 22 weight-% mannitol, from 25 to 35 weight-%, preferably from 30 to 32 weight-% microcrystalline cellulose, from 2 to 10 weight-%, preferably from 4 to 6 weight-% croscarmellose sodium, from 0.2 to 2 weight-%, preferably from 0.5 to 1.5 weight-% colloidal silica, from 0.5 to 5 weight-%, preferably from 1 to 1.5 weight-% magnesium stearate, in each based on the total weight of the solid pharmaceutical composition.
XV. The solid pharmaceutical composition of any one of embodiments X to XIV, comprising from 40 to 41 weight-% of the solid composition, from 20 to 21 weight-% mannitol, from 30 to 31 weight-% microcrystalline cellulose, from 4.5 to 5.5 weight-% croscarmellose sodium, from 0.5 to 1.0 weight-% colloidal silica, from 1.0 to 2.0 weight-% magnesium stearate, in each based on the total weight of the solid pharmaceutical composition, wherein the individual contents add up to 100%.
XVI. The solid pharmaceutical composition of any one of embodiments I to XV, wherein the solid composition is prepared by a process comprising embedding sofosbuvir in a matrix comprising, preferably consisting of the at least one pharmaceutically acceptable matrix compound, wherein said embedding comprises melting the at least one pharmaceutically acceptable matrix compound together with the sofosbuvir in solid form, preferably by a hot-melt method, more preferably by a hot-melt extrusion method.
XVII. The solid pharmaceutical composition of embodiment XVI, wherein the sofosbuvir in solid form is sofosbuvir in at least one crystalline form or in amorphous form or as a mixture of at least one crystalline form and amorphous form, wherein preferably at least 95 weight-%, preferably at least 99 weight-%, more preferably at least 99.9 weight-% of the sofosbuvir are present in at least one crystalline form.
XVIII. The solid pharmaceutical composition of any embodiments XVI or XVII, wherein at least 99 weight-%, preferably at least 99.5 weight-%, more preferably at least 99.9 weight-% of the sofosbuvir comprised in the solid composition are present in amorphous form directly after preparing the solid composition.
XIX. The solid pharmaceutical composition of any one of embodiments I to XVIII, obtainable or obtained by a process comprising
   (i) preparing a solid composition comprising sofosbuvir and at least one pharmaceutically acceptable matrix compound, wherein at least 99 weight-% of the sofosbuvir comprised in the solid composition are present in amorphous form, at least 99 weight-% of the solid composition consist of the sofosbuvir and the at least one matrix compound, wherein the solid composition contains the sofosbuvir in an amount of at least 25 weight-%, or at least 30 weight-%, or at least 35 weight-%, or at least 40 weight-% or at least 50 weight-% or preferably at least 55 weight-% based on the combined weight of the sofosbuvir and the at least one matrix compound,
   (ii) directly processing the solid composition obtained from (i) to the pharmaceutical composition.
XX. The solid pharmaceutical composition of any one of embodiments X to XIX, having a moisture stability of at least 95%, preferably at least 98%, more preferably at least 99%, wherein the moisture stability is defined as the amount of solid amorphous sofosbuvir which is present in the solid pharmaceutical composition after having been exposed to a relative humidity of 75% at 40° C. for 8 weeks, relative to the amount of solid amorphous sofosbuvir which is present in the solid pharmaceutical composition before said exposure, said moisture stability preferably being determined as described in Reference Example 2 herein.
XXI. The solid composition of any one of embodiments I to XX for use in a method for treating hepatitis C in a human.
XXII. The solid composition of any one of embodiments I to XX for treating hepatitis C in a human.
XXIII. A solid composition comprising sofosbuvir according to formula (I)

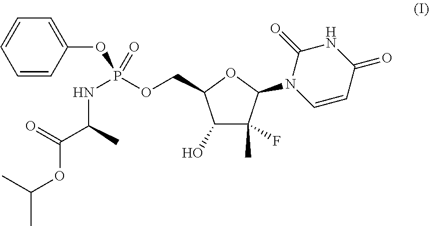 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

and at least one pharmaceutically acceptable matrix compound, wherein at least 99 weight-% of the sofosbuvir comprised in the solid composition are present in amorphous form, at least 99 weight-% of the solid composition consist of the sofosbuvir and the at least one matrix compound, wherein the solid composition contains the sofosbuvir in an amount of at least 25 weight-%, or at least 30 weight-%, or at least 35 weight-%, or at least 40 weight-% or at least 50 weight-% or preferably of at least 55 weight-% based on the combined weight of the sofosbuvir and the at least one matrix compound,
wherein the at least one pharmaceutically acceptable matrix compound comprises, preferably consists of, at least one vinyl pyrrolidone-vinyl acetate copolymer.
XXIV. The solid composition of embodiment XXIII, wherein the at least one pharmaceutically acceptable matrix compound comprises, preferably consists of, copovidone.
XXV. The solid composition of embodiments XXIII or XXIV, wherein the weight average molecular weight (M_{w}) of the at least one pharmaceutically acceptable matrix compound is in the range of from 20 to 100 kDa, preferably in the range of from 30 to 85 kDa, more preferably in the range of from 40 to 75 kDa.
XXVI. The solid composition of any one of embodiments XXIII to XXV, wherein at least 99.5 weight-%, preferably at least 99.9 weight-% of the solid composition consist of the sofosbuvir and the at least one matrix compound.
XXVII. The solid composition of any one of embodiments XXIII to XXVI, wherein the solid composition comprises less than 0.1 weight-%, preferably less than 0.01 weight-%, more preferably less than 0.001 weight-% of a surfactant.
XXVIII. The solid composition of any one of embodiments XXIII to XXVII, wherein in the adsorption-desorption isotherm of the at least one pharmaceutically acceptable matrix compound, ΔΔm/% which is defined as the mass difference Δm(desorption)/% at 75% relative humidity and 25° C. minus the mass difference Δm(adsorption)/% at 75% relative humidity and 25° C., determined according to dynamic vapor sorption measurement, in particular determined according to dynamic vapor sorption measurement as described in Reference Example 1 herein, is in the range of from −0.1≤ΔΔm/%<0, preferably in the range of from −0.1≤ΔΔm/%≤0.001.
XXIX. The solid composition of any one embodiments XXIII to XXVIII, wherein the solid composition comprises the sofosbuvir in an amount in the range of from 55 to 95 weight-%, preferably from 65 to 90 weight-%, more preferably from 75 to 85 weight-%, based on the combined weight of the sofosbuvir and the at least one matrix compound.
XXX. The solid composition of any one of embodiments XXIII to XXIX, wherein the solid composition is a solid dispersion.
XXXI. The solid composition of any one of embodiments XXIII to XXX for use in a method for treating hepatitis C in a human.
XXXII. The solid composition of any one of embodiments XXIII to XXX for treating hepatitis C in a human.

According to a further aspect, the present invention is illustrated by the following embodiments and combinations of embodiments resulting from the given dependencies and back-references:
1. A solid pharmaceutical composition wherein the solid pharmaceutical composition comprises sofosbuvir according to formula (I)

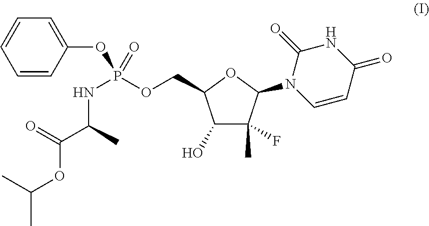 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

at least one pharmaceutically acceptable matrix compound and one or more pharmaceutically acceptable excipient(s), wherein at least 99 weight-% of the sofosbuvir comprised in the solid pharmaceutical composition are present in amorphous form, and at least 99 weight-% of the solid pharmaceutical composition consist of the sofosbuvir, the at least one matrix compound and the one or more pharmaceutically acceptable excipient(s).
2. The solid pharmaceutical composition of embodiment 1, wherein the solid pharmaceutical composition comprises the sofosbuvir in an amount in the range of from 15 to 95 weight-%, preferably from 20 to 70 weight-%, preferably from 20 to 45 weight-%, more preferably from 20 to 40 weight-%, more preferably 30 to 40 weight-%, more preferably 30 weight-%, based on the total weight of the solid pharmaceutical composition.
3. The solid pharmaceutical composition of embodiment 1 or 2, wherein the at least one matrix compound is selected from the group consisting of hydrophilic polymers, silicon-based inorganic adsorbents and a combination of two or more thereof.
4. The solid pharmaceutical composition of embodiment 3, wherein the silicon-based inorganic adsorbents include, preferably are, one or more of silica and silicates.
5. The solid pharmaceutical composition of embodiment 3 or 4, wherein the at least one silicon-based inorganic adsorbent has an oil absorbance in the range of from 1.0 to 5.0 ml/g, preferably in the range of from 1.5 to 4.0 ml/g.
6. The solid pharmaceutical composition of any one of embodiments 3 to 5, wherein the at least one silicon-based inorganic adsorbent has a bulk density in the range of from 10 to 600 g/ml, preferably in the range of from 30 to 500 g/ml, more preferably in the range of from 50 to 300 g/ml, more preferably in the range of from 50 to 200 g/ml.
7. The solid pharmaceutical composition of any one of embodiments 5 to 7, wherein the at least one silicon-based inorganic adsorbent is selected from the group consisting of silica, silicates, and a combination of two or more thereof, wherein the silica is preferably selected from the group consisting of fumed silica, precipitated silica, gel silica, colloidal silica, and a combination of two or more thereof, and wherein the silicates are preferably aluminosilicates preferably comprising at least one alkali metal element and/or at least one alkaline earth metal element, more preferably at least one alkaline earth metal element, more preferably magnesium, wherein more preferably, at least 90 weight-%, more preferably at least 95 weight-%, more preferably at least 99 weight-% of the at least one silicon-based inorganic adsorbent are present in amorphous form.
8. The solid pharmaceutical composition of embodiment 3, wherein the hydrophilic polymers include, preferably are, one or more of polysaccharides, preferably cellulose derivatives such as hydroxyalkylalkylcelullose, polyvinylpyrrolidones, polyethylene glycols, polyethylene glycol based copolymers, polyacrylic acids, salts of polyacrylic acids, polyvinyl alcohols, polyacrylamide copolymers, methacrylic acid copolymers, methacrylate copolymers, pectines, chitin derivatives, chitosan derivatives, polyphosphates, polyoxazolines.
9. The solid pharmaceutical composition of embodiment 3 or 8, wherein the at least one matrix compound is selected from the group consisting of hydrophilic water-soluble polymers and a combination of two or more thereof.
10. The solid pharmaceutical composition of embodiment 9, wherein the at least one hydrophilic water-soluble polymer has a solubility in water of at least 10 g/l, preferably of at least 20 g/l, more preferably of at least 30 g/l, in each case at 23° C. at atmospheric pressure.
11. The solid pharmaceutical composition of embodiment 9 or 10, wherein the at least one hydrophilic water-soluble polymer comprises, preferably consists of, at least one vinyl pyrrolidone-vinyl acetate copolymer.
12. The solid pharmaceutical composition of any one of embodiments 9 to 11, wherein the at least one hydrophilic water-soluble polymer comprises, preferably consists of, copovidone.
13. The solid pharmaceutical composition of any one of embodiments 9 to 12, wherein the weight average molecular weight (M_{w}) of the at least one hydrophilic water-soluble polymer is in the range of from 20 to 100 kDa, preferably in the range of from 30 to 85 kDa, more preferably in the range of from 40 to 75 kDa.
14. The solid pharmaceutical composition of embodiment 3 or 8, wherein the at least one hydrophilic water-soluble polymer comprises, preferably consists of a cellulose derivative selected from the group consisting of hydroxyalkylalkylcelluloses and a mixture of two or more thereof, the at least one hydrophilic water-soluble polymer preferably comprising, more preferably consisting of, hydroxypropylmethylcellulose (HPMC).
15. The solid pharmaceutical composition of embodiment 14, wherein the cellulose derivative has a degree of substitution (DS) in the range of from 0.3 to 2.8, preferably in the range of from 0.6 to 2.5, more preferably in the range of from 1.0 to 2.3, more preferably in the range of from 1.3 to 2.0.
16. The solid pharmaceutical composition of embodiment 14 or 15, wherein the weight average molecular weight (M_{w}) of the cellulose derivative is in the range of from 7 to 225 kDa, preferably in the range of from 7 to 100 kDa, more preferably in the range of from 7 to 30 kDa.
17. The solid pharmaceutical composition of any one of embodiments 1 to 16, wherein at least 99.5 weight-%, preferably at least 99.9 weight-% of the sofosbuvir comprised in the composition are present in amorphous form.
18. The solid pharmaceutical composition of any one of embodiments 1 to 17, wherein at least 99.5 weight-%, preferably at least 99.9 weight-% of the solid pharmaceutical composition consist of the sofosbuvir, the at least one matrix compound and one or more pharmaceutically acceptable excipient(s).
19. The solid pharmaceutical composition of any one of embodiments 1 to 18, wherein from 30 to 60 weight-% or from 30 to 50 weight-% or from 35 to 50 weight-% of the solid pharmaceutical composition consist of sofosbuvir based on the total weight of the solid pharmaceutical composition.
20. The solid pharmaceutical composition of any one of embodiments 1 to 19, wherein from 30 to 45 weight-%, preferably from 30 to 40 weight % of the solid pharmaceutical composition consist of sofosbuvir based on the total weight of the solid pharmaceutical composition.
21. The solid pharmaceutical composition of any one of embodiments 1 to 20, wherein from 3 to 15 weight-%, more preferably from 3 to 13 weight-%, more preferably 3 to 5 weight-% of the solid pharmaceutical composition consist of the at least one pharmaceutically acceptable matrix compound based on the total weight of the solid pharmaceutical composition.
22. The solid pharmaceutical composition of any one of embodiments 1 to 21, wherein the solid pharmaceutical composition comprises less than 0.1 weight-%, preferably less than 0.01 weight-%, more preferably less than 0.001 weight-% of a surfactant.
23. The solid pharmaceutical composition of any one of embodiments 1 to 22, wherein the solid pharmaceutical composition is a solid dispersion.
24. The solid pharmaceutical composition of any one of embodiments 1 to 23, being an oral dosage form, preferably a tablet.
25. The solid pharmaceutical composition of any one of embodiments 1 to 24, further comprising, in addition to the sofosbuvir, one or more further HCV agents including one or more of ledipasvir according to formula (II)

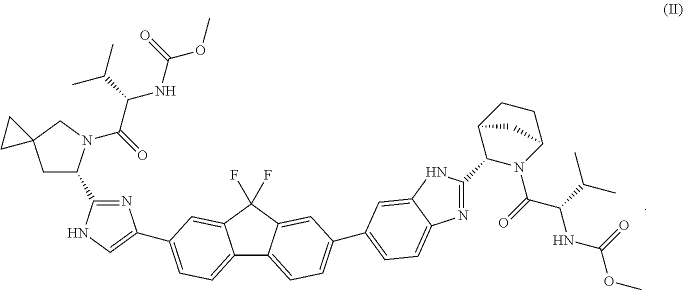 [SMILES: COC(=O)N[C@H](C(=O)N1CC2(CC2)C[C@H]1c1nc(-c2ccc3c(c2)C(F)(F)c2cc(-c4ccc5nc([C@@H]6[C@H]7CC[C@H](C7)N6C(=O)[C@@H](NC(=O)OC)C(C)C)[nH]c5c4)ccc2-3)c[nH]1)C(C)C]

26. The solid pharmaceutical composition of any one of embodiments 1 to 25, wherein the solid pharmaceutical composition consists of sofosbuvir, the at least one pharmaceutically acceptable matrix compound, the one or more pharmaceutically acceptable excipient(s) and optionally the one or more further HCV agents.
27. The solid pharmaceutical composition of embodiment 26, wherein at least one of the one or more pharmaceutically acceptable excipient(s) is different from the at least one pharmaceutically acceptable matrix compound comprised in the solid pharmaceutical composition.
28. The solid pharmaceutical composition of embodiment 27, wherein the at least one of the one or more pharmaceutically acceptable excipient(s) is not a pharmaceutically acceptable matrix compound as defined in any one of embodiments 3 to 16.
29. The solid pharmaceutical composition of any one of embodiments 1 to 28, wherein the one or more pharmaceutically acceptable excipient(s) comprise one or more of at least one of a diluent, at least one disintegrant, at least one glidant, optionally at least one lubricant, and combinations of two or more thereof.
30. The solid pharmaceutical composition of embodiment 29, wherein the at least one diluent includes one or more of calcium carbonate, dicalcium phosphate, dry starch, calcium sulfate, cellulose, compressible sugars, confectioner's sugar, dextrates, dextrin, dextrose, dibasic calcium phosphate dihydrate, glyceryl palmitostearate, hydrogenated vegetable oil, inositol, kaolin, lactose, magnesium carbonate, magnesium oxide, maltodextrin, mannitol, microcrystalline cellulose, polymethacrylates, potassium chloride, powdered cellulose, powdered sugar, pregelatinized starch, sodium chloride, sorbitol, starch, sucrose, sugar spheres, talc, tribasic calcium phosphate.
31. The solid pharmaceutical composition of embodiment 30, wherein the at least one diluent is mannitol.
32. The solid pharmaceutical composition of embodiment 29 or 30, wherein the at least one disintegrant includes one or more of agar, alginic acid, bentonite, carboxymethylcellulose calcium, carboxymethylcellulose sodium, carboxymethylcellulose, cellulose, a cation exchange resin, cellulose, gums, citrus pulp, colloidal silicon dioxide, corn starch, croscarmellose sodium, crospovidone, guar gum, hydrous aluminum silicate, an ion exchange resin such as polyacrin potassium, magnesium aluminum silicate, methyl cellulose, microcrystalline cellulose, modified cellulose gum, modified corn starch, montmorillonite clay, natural sponge, polyacrilin potassium, potato starch, powdered cellulose, povidone, pregelatinized starch, sodium alginate, sodium bicarbonate optionally in admixture with one or more acidulants, sodium starch glycolate, starch, silicates.
33. The solid pharmaceutical composition of embodiment 31, wherein the at least one disintegrant is selected from croscarmellose sodium, such as Ac-Di-Sol® croscarmellose sodium.
34. The solid pharmaceutical composition of any one of embodiments 29 to 33, wherein the at least one glidant includes one or more of colloidal silicon dioxide, talc, starch, starch derivatives.
35. The solid pharmaceutical composition of embodiment 34, wherein the at least one glidant is colloidal silicon dioxide.
36. The solid pharmaceutical composition of any one of embodiments 29 to 35, wherein the at least one lubricant includes one or more of calcium stearate, glyceryl monostearate, glyceryl palmitostearate, hydrogenated castor oil, hydrogenated vegetable oil, light mineral oil, magnesium stearate, mineral oil, polyethylene glycol, sodium benzoate, sodium lauryl sulfate, sodium stearyl fumarate, stearic acid, talc, zinc stearate.
37. The solid pharmaceutical composition of embodiment 36, wherein the at least one glidant is magnesium stearate.
38. The solid pharmaceutical composition of any one of embodiments 29 to 37, wherein the one or more pharmaceutically acceptable excipient(s) comprise, preferably are, a combination of at least one a diluent, at least one disintegrant, at least one glidant, and optionally at least one lubricant.
39. The solid pharmaceutical composition of any one of embodiments 29 to 38, comprising
   from 20 to 30 weight-%, preferably from 22 to 26 weight-% diluent,
   from 20 to 30 weight-%, preferably from 24 to 28 weight-% first disintegrant,
   from 2 to 6 weight-%, preferably from 2.5 to 5.5 weight-% second disintegrant,
   from 0.5 to 7 weight-%, preferably from 0.8 to 5 weight-% glidant,
   optionally from 1 to 6 weight-%, preferably from 1.4 to 2 weight-% lubricant,
   in each based on the total weight of the solid pharmaceutical composition.
40. The solid pharmaceutical composition of any one of embodiments 29 to 39, comprising
   from 30 to 45 weight-%, preferably from 30 to 40 weight-% sofosbuvir,
   from 5 to 15 weight-%, preferably from 7 to 13 weight-% of the pharmaceutically acceptable matrix compound,
   from 20 to 30 weight-%, preferably from 22 to 26 weight-% diluent,
   from 20 to 30 weight-%, preferably from 24 to 28 weight-% first disintegrant,
   from 2 to 6 weight-%, preferably from 2.5 to 5.5 weight-% second disintegrant,
   from 0.5 to 7 weight-%, preferably from 0.8 to 5 weight-% glidant,
   optionally from 1 to 6 weight-%, preferably from 1.4 to 2 weight-% lubricant,
   in each based on the total weight of the solid pharmaceutical composition.
41. The solid pharmaceutical composition of any one of embodiments 29 to 42, wherein the one or more pharmaceutically acceptable excipient(s) comprise, preferably are, a combination of mannitol, microcrystalline cellulose, croscarmellose sodium, silica dioxide, magnesium stearate.
42. The solid pharmaceutical composition of embodiment 41, comprising
   from 20 to 30 weight-%, preferably from 22 to 26 weight-% mannitol,
   from 20 to 30 weight-%, preferably from 24 to 28 weight-% microcrystalline cellulose,
   from 2 to 6 weight-%, preferably from 2.5 to 5.5 weight-% croscarmellose sodium, from 0.5 to 7 weight-%, preferably from 0.8 to 5 weight-% silica dioxide,
   optionally from 1 to 6 weight-%, preferably from 1.4 to 2 weight-% magnesium stearate,
   in each based on the total weight of the solid pharmaceutical composition.
43. The solid pharmaceutical composition of embodiment 42, comprising
   from 30 to 45 weight-%, preferably from 30 to 40 weight-% sofosbuvir,
   from 5 to 15 weight-%, preferably from 7 to 13 weight-% copovidone,
   from 20 to 30 weight-%, preferably from 22 to 26 weight-% mannitol,
   from 20 to 30 weight-%, preferably from 24 to 28 weight-% microcrystalline cellulose,
   from 2 to 6 weight-%, preferably from 2.5 to 5.5 weight-% croscarmellose sodium, from 0.5 to 7 weight-%, preferably from 0.8 to 5 weight-% silica dioxide,
   optionally from 1 to 6 weight-%, preferably from 1.4 to 2 weight-% magnesium stearate,
   in each based on the total weight of the solid pharmaceutical composition.
44. The solid pharmaceutical composition of embodiment 41 or 42, comprising
   from 30 to 40 weight-% sofosbuvir,
   from 7 to 13 weight-% copovidone,
   from 22 to 26 weight-% mannitol,
   from 24 to 28 weight-% microcrystalline cellulose,
   from 2.5 to 5.5 weight-% croscarmellose sodium,
   from 0.8 to 5 weight-% colloidal silica,
   optionally from 1.4 to 2 weight-% magnesium stearate,
   in each based on the total weight of the solid pharmaceutical composition, wherein the individual contents add up to 100%.
45. The solid pharmaceutical composition of any one of embodiments 1 to 44, which is a tablet.
46. The solid pharmaceutical composition of embodiment 45, wherein the tablet is a coated tablet.
47. The solid pharmaceutical composition of embodiments 45 or 46, wherein the tablet has a weight in the range of from 900 mg to 2300 mg, preferably from 1000 mg to 2000 mg.
48. The solid pharmaceutical composition of any one of embodiments 1 to 47, obtainable or obtained by a process comprising
   (i) preparing a mixture comprising sofosbuvir, at least one pharmaceutically acceptable matrix compound and one or more pharmaceutically acceptable excipient(s), wherein at least 99 weight-% of the mixture consist of the sofosbuvir, the at least one matrix compound and the one or more pharmaceutically acceptable excipient(s);
   (ii) processing the mixture obtained from (i) to the solid pharmaceutical composition, wherein preferably the solid pharmaceutical composition is a tablet.
49. The solid pharmaceutical composition of embodiment 48, said process comprising
   (ii) directly processing the mixture obtained from (i) to the pharmaceutical composition.
50. The solid pharmaceutical composition of embodiment 48 or 49, wherein after (i) and before (ii), the mixture obtained from (i) is not subjected to any modification.
51. The solid pharmaceutical composition of any one of embodiments 48 to 50, wherein the mixture obtained from (i) is processed to the pharmaceutical composition according to (ii) at most 168 h, preferably at most 72 h, more preferably at most 24 h after having been obtained from (i), wherein during this period of time, the mixture is preferably not subjected to stress conditions of 30° C. and a relative humidity of 75%, more preferably stored under ambient conditions.
52. The solid pharmaceutical composition of any one of embodiments 48 to 51, wherein (ii) comprise one or more of wet granulation, dry granulation, compression, melting extrusion of the mixture of (i).
53. The solid pharmaceutical composition of any one of embodiments 48 to 52, wherein (ii) comprise melting extruding the mixture of (i), preferably hot melting extruding the mixture of (i).
54. The solid pharmaceutical composition of any one of embodiments 1 to 53, wherein the solid pharmaceutical composition is prepared by a process comprising embedding sofosbuvir in a matrix comprising, preferably consisting of the at least one pharmaceutically acceptable matrix compound and one or more pharmaceutically acceptable excipient(s), wherein said embedding comprises melt extruding the at least one pharmaceutically acceptable matrix compound and the one or more pharmaceutically acceptable excipient(s) together with the sofosbuvir.
55. The solid pharmaceutical composition of embodiment 1 to 54, wherein the solid pharmaceutical composition is prepared by a process comprising embedding sofosbuvir in a matrix comprising, preferably consisting of the at least one pharmaceutically acceptable matrix compound and one or more pharmaceutically acceptable excipient(s), by melt extruding the at least one pharmaceutically acceptable matrix compound and one or more pharmaceutically acceptable excipient(s) in solid form together with the sofosbuvir in solid form, preferably by a hot-melt extrusion method.
56. The solid pharmaceutical composition of any one of embodiments 52 to 55, wherein the sofosbuvir before melt extrusion is sofosbuvir in at least one crystalline form or in amorphous form or as a mixture of at least one crystalline form and amorphous form, wherein preferably at least 95 weight-%, preferably at least 99 weight-%, more preferably at least 99.9 weight-% of the sofosbuvir are present in at least one crystalline form, preferably crystalline form 1.
57. The solid pharmaceutical composition of any one of embodiments 1 to 56 wherein at least 99 weight-%, preferably at least 99.5 weight-%, more preferably at least 99.9 weight-% of the sofosbuvir comprised in the solid pharmaceutical composition are present in amorphous form.
58. The solid pharmaceutical composition of any one of embodiments 53 to 77, wherein the melt extrusion is carried out at a temperature of at least 100° C., preferably at least 150° C.
59. The solid pharmaceutical composition of any one of embodiments 1 to 58, obtainable or obtained by a process comprising
   (i′) preparing a mixture of sofosbuvir and the at least one pharmaceutically acceptable matrix compound and at least one solvent;
   (ii′) subjecting the mixture of (i′) to drying, preferably by lyophilizing the mixture of (i′) or spray-drying the mixture of (i′), obtaining a dried, preferably lyophilize or spray-dried mixture-1;
   (iii′) mixing the mixture-1 of (ii′) with the one or more pharmaceutically acceptable excipient(s), obtaining a mixture-2;
   (iv′) processing the mixture-2 of (iii′) to the solid pharmaceutical composition.
60. The solid pharmaceutical composition of embodiment 59, wherein the mixture of (i′), is a solution or a dispersion or a suspension.
61. The solid pharmaceutical composition of any one of embodiments 1 to 60, wherein the solid pharmaceutical composition is obtained or obtainable by a process comprising embedding sofosbuvir in a matrix comprising, preferably consisting of the at least one pharmaceutically acceptable matrix compound, wherein said embedding comprises preparing a mixture of the sofosbuvir, the at least one pharmaceutically acceptable matrix compound and at least one solvent preferably selected from the group consisting of an organic solvent, and a combination of two or more thereof, more preferably selected from the group consisting of a C1-C2 halogenated hydrocarbon, a C1-C4 alcohol, a C3-C6 ketone, a C2-C6 ether, a C3-C5 ester, a combination of two or more thereof and a combination of one or more thereof with water, and subjecting said mixture to drying, preferably by lyophilizing the solution or spray-drying the mixture.
62. The solid pharmaceutical composition of embodiment 61, wherein the process further comprises mixing to the dried, preferably lyophilize or spray-dried solution with the one or more pharmaceutically acceptable excipient(s) to the dried, preferably lyophilized or spray-dried solution, obtaining a mixture and processing said mixture to the solid pharmaceutical composition.
63. The solid pharmaceutical composition of any one of embodiments 59 to 62, wherein the organic solvent is selected from the group consisting of C3-C6 ketones such as acetone, C1-C2 halogenated hydrocarbons such as CH₂Cl₂, C1-C4 alcohols such as methanol, C2-C6 ethers, C3-C5 esters such as ethylacetate, and a combination of two or more thereof and a combination of one and water, more preferably from the group consisting of C1-C4 alcohols such as methanol, C3-C6 ketones such as acetone, and a combination of two or more thereof, wherein more preferably, the at least one solvent comprises, more preferably consists of, and C1-C4 alcohol, preferably methanol, or comprises, more preferably consists of, acetone.
64. The solid pharmaceutical composition of any one of embodiments 1 to 63, for use in a method for treating hepatitis C in a human.
65. The solid pharmaceutical composition of any one of embodiments 1 to 64, for treating hepatitis C in a human.
66. A process for preparing a solid pharmaceutical composition according to any one of embodiments 1 to 65, comprising
   (i) preparing a mixture comprising sofosbuvir, at least one pharmaceutically acceptable matrix compound and one or more pharmaceutically acceptable excipient(s), wherein at least 99 weight-% of the mixture consist of the sofosbuvir, the at least one matrix compound and the one or more pharmaceutically acceptable excipient(s);
   (ii) processing the mixture obtained from (i) to the solid pharmaceutical composition.
67. The process of embodiment 66, said processing comprising
   (ii) directly processing the mixture obtained from (i) to the pharmaceutical composition.
68. The process of embodiment 66 or 67, wherein after (i) and before (ii), the mixture obtained from (i) is not subjected to any modification.
69. The process of any one of embodiments 66 to 68, wherein (ii) comprise one or more of wet granulation, dry granulation, compression, melting extrusion of the mixture of (i).
70. The process of any one of embodiments 66 to 69, wherein (ii) comprise melting extruding the mixture of (i), preferably hot melting extruding the mixture of (i).
71. The process of any one of embodiments 66 to 70, wherein the mixture obtained from (i) is melt extruded to the pharmaceutical composition according to (ii) at most 168 h, preferably at most 72 h, more preferably at most 24 h after having been obtained from (i), wherein during this period of time, the solid pharmaceutical composition is preferably not subjected to stress conditions of 30° C. and a relative humidity of 75%, more preferably stored under ambient conditions.
72. The process of any one of embodiments 66 to 71, wherein the solid pharmaceutical composition is a solid dispersion.
73. The process of any one of embodiments 66 to 72, wherein in (i) the weight ratio of the sofosbuvir relative to the at least one matrix compound is at least 10:1, preferably in the range of from 6:1 to 1:1 more preferably in the range of from 5:1 to 2:1, more preferably in the range of from 4.5:1 to 2.6:1.
74. The process of any one of embodiments 66 to 73, wherein (i) and (ii) comprise embedding the sofosbuvir in a matrix comprising, preferably consisting of the at least one pharmaceutically acceptable matrix compound and one or more pharmaceutically acceptable excipient(s), wherein said embedding preferably comprises melting extruding the at least one pharmaceutically acceptable matrix compound and the one or more pharmaceutically acceptable excipient(s) together with the sofosbuvir.
75. The process of embodiment 66 to 73, wherein (i) and (ii) comprise embedding sofosbuvir in a matrix comprising, preferably consisting of the at least one pharmaceutically acceptable matrix compound and at least one pharmaceutical acceptable excipient, by melting extruding the at least one pharmaceutically acceptable matrix compound in solid form and the one or more pharmaceutical acceptable excipient(s) in solid form together with the sofosbuvir in solid form, preferably by a hot-melt extrusion method.
76. The process of any one of embodiments 66 to 75, wherein (ii) comprises melting extruding at a temperature in the range of from 75 to 175° C., preferably in the range of from 90 to 150° C.
77. The process of any one of embodiments 66 to 76, wherein the process after the melting extruding according to (ii), comprises cooling, preferably to a temperature in the range of from 10 to 40° C., preferably in the range of from 20 to 30° C.
78. The process of any one of embodiments 66 to 77, wherein the sofosbuvir in solid form before processing, preferably melting extruding is sofosbuvir in at least one crystalline form or in amorphous form or as a mixture of at least one crystalline form and amorphous form, wherein preferably at least 95 weight-%, preferably at least 99 weight-%, more preferably at least 99.9 weight-% of the sofosbuvir are present in at least one crystalline form.
79. The process of any one of embodiments 66 to 78, wherein at least 99 weight-%, preferably at least 99.5 weight-%, more preferably at least 99.9 weight-% of the sofosbuvir comprised in the solid pharmaceutical composition are present in amorphous form directly after preparing the solid composition.
80. A process for preparing a solid pharmaceutical composition according to any one of embodiments 1 to 44, comprising
   (i′) preparing a mixture of sofosbuvir and the at least one pharmaceutically acceptable matrix compound and at least one solvent;
   (ii′) subjecting the mixture of (i′) to drying, preferably by lyophilizing the mixture of (i′) or spray-drying the mixture of (i′), obtaining a dried, preferably lyophilize or spray-dried mixture-1;
   (iii′) mixing the mixture-1 of (ii′) with the one or more pharmaceutically acceptable excipient(s), obtaining a mixture-2;
   (iv′) processing the mixture-2 of (iii′) to the solid pharmaceutical composition.
81. The process of embodiment 80, wherein the mixture of (i′) is a solution or a dispersion or a suspension.
82. The process of embodiment 80 or 81, wherein (i′) and (ii′) comprise embedding sofosbuvir in a matrix comprising, preferably consisting of the at least one pharmaceutically acceptable matrix compound, wherein said embedding comprises preparing a mixture of the sofosbuvir, the at least one pharmaceutically acceptable matrix compound and at least one solvent and subjecting said mixture to drying, preferably by lyophilizing the mixture or spray-drying the mixture.
83. The process of embodiments 80 or 81, wherein the at least one solvent is selected from the group consisting of an organic solvent, and a combination of two or more thereof, wherein the organic solvent is preferably selected from the group consisting of a C1-C2 halogenated hydrocarbon, a C1-C4 alcohol, a C3-C6 ketone, a C2-C6 ether, a C3-C5 ester, a combination of two or more thereof and a combination of one or more thereof with water.
84. The process of embodiment 83, wherein the at least one solvent is selected from the group consisting of C1-C4 alcohols, C1-C2 halogenated hydrocarbons, C3-C6 ketones, C2-C6 ethers, C3-C5 esters, and a combination of two or more thereof.
85. The process of embodiment 83 or 84, wherein the at least one solvent is selected from the group consisting of acetone, CH₂Cl₂ and methanol, preferably acetone.
86. The process of any one of embodiments 82 to 85, wherein the embedding comprises subjecting the solution to drying, preferably by lyophilizing the solution or spray-drying the solution.
87. The process of any one of embodiments 66 to 87, wherein the at least one matrix compound is selected from the group consisting of hydrophilic water-soluble polymers, silicon-based inorganic adsorbents and a combination of two or more thereof.
87. The process of any one of embodiments 66 to 87, wherein the at least one matrix compound is selected from the group consisting of silicon-based inorganic adsorbents and a combination of two or more thereof and wherein the embedding comprises dispersing the at least one matrix compound in the solution.
88. The process of embodiment 87, wherein the at least one matrix compound has a pH in the range of from 6.0 to 9.0, preferably in the range of from 6.5 to 8.5, more preferably in the range of from 7.0 to 8.0.
89. The process of any one of embodiments 82 to 77, wherein the embedding comprises subjecting the dispersion to drying, preferably filtrating the dispersion or evaporating the dispersion, preferably followed by vacuum drying.
90. The process of any one of embodiments 66 to 89, wherein the solid pharmaceutical composition comprises the sofosbuvir in an amount in the range of from 15 to 95 weight-%, preferably from 30 to 45 weight-%, more preferably from 30 to 40 weight-%, based on the total weight of the solid pharmaceutical composition.
91. The process of any one of embodiments 66 to 90, wherein the at least one matrix compound is selected from the group consisting of hydrophilic polymers, silicon-based inorganic adsorbents and a combination of two or more thereof.
92. The process of any one of embodiments 66 to 91, wherein the silicon-based inorganic adsorbents include, preferably are, one or more of silica and silicates.
93. The process of any one of embodiments 66 to 92, wherein the at least one silicon-based inorganic adsorbent has an oil absorbance in the range of from 1.0 to 5.0 ml/g, preferably in the range of from 1.5 to 4.0 ml/g.
94. The process of any one of embodiments 66 to 93, wherein the at least one silicon-based inorganic adsorbent has a bulk density in the range of from 10 to 600 g/ml, preferably in the range of from 30 to 500 g/ml, more preferably in the range of from 50 to 300 g/ml, more preferably in the range of from 50 to 200 g/ml.
95. The process of any one of embodiments 66 to 94, wherein the at least one silicon-based inorganic adsorbent is selected from the group consisting of silica, silicates, and a combination of two or more thereof, wherein the silica is preferably selected from the group consisting of fumed silica, precipitated silica, gel silica, colloidal silica, and a combination of two or more thereof, and wherein the silicates are preferably aluminosilicates preferably comprising at least one alkali metal element and/or at least one alkaline earth metal element, more preferably at least one alkaline earth metal element, more preferably magnesium, wherein more preferably, at least 90 weight-%, more preferably at least 95 weight-%, more preferably at least 99 weight-% of the at least one silicon-based inorganic adsorbent are present in amorphous form.
96. The process of embodiment 95, wherein the hydrophilic polymers include, preferably are, one or more of polysaccharides, preferably cellulose derivatives such as hydroxyalkylalkylcelullose, polyvinylpyrrolidones, polyethylene glycols, polyethylene glycol based copolymers, polyacrylic acids, salts of polyacrylic acids, polyvinyl alcohols, polyacrylamide copolymers, methacrylic acid copolymers, methacrylate copolymers, pectines, chitin derivatives, chitosan derivatives, polyphosphates, polyoxazolines.
97. The process of any one of embodiments 66 to 96 wherein the at least one matrix compound is selected from the group consisting of hydrophilic water-soluble polymers and a combination of two or more thereof.
98. The process of embodiment 97, wherein the at least one hydrophilic water-soluble polymer comprises, preferably consists of, at least one vinyl pyrrolidone-vinyl acetate copolymer.
99. The process of any one of embodiments 97 or 98, wherein the at least one hydrophilic water-soluble polymer comprises, preferably consists of, copovidone.
100. The process of any one of embodiments 97 to 99, wherein the weight average molecular weight (M_{w}) of the at least one hydrophilic water-soluble polymer is in the range of from 20 to 100 kDa, preferably in the range of from 30 to 85 kDa, more preferably in the range of from 40 to 75 kDa.
101. The process of embodiment 97, wherein the at least one hydrophilic water-soluble polymer comprises, preferably consists of a cellulose derivative selected from the group consisting of hydroxyalkylalkylcelluloses and a mixture of two or more thereof, the at least one hydrophilic water-soluble polymer preferably comprising, more preferably consisting of, hydroxypropylmethylcellulose (HPMC).
102. The process of embodiment 101, wherein the cellulose derivative has a degree of substitution (DS) in the range of from 0.3 to 2.8, preferably in the range of from 0.6 to 2.5, more preferably in the range of from 1.0 to 2.3, more preferably in the range of from 1.3 to 2.0.
103 The process of embodiment 101 or 102, wherein the weight average molecular weight (M_{w}) of the cellulose derivative is in the range of from 7 to 225 kDa, preferably in the range of from 7 to 100 kDa, more preferably in the range of from 7 to 30 kDa.
104. The process of any one of embodiments 66 to 104, wherein at least 99.5 weight-%, preferably at least 99.9 weight-% of the sofosbuvir comprised in the composition are present in amorphous form.
105. The process of any one of embodiments 66 to 104 wherein at least 99.5 weight-%, preferably at least 99.9 weight-% of the solid pharmaceutical composition consist of the sofosbuvir the at least one matrix compound and one or more pharmaceutically acceptable excipient(s).
106. The process of any one of embodiments 66 to 105 wherein from 30 to 60 weight-% or from 35 to 55 weight-% or from 40 to 50 weight-% of the solid pharmaceutical composition consist of sofosbuvir based on the total weight of the solid pharmaceutical composition.
107. The process of any one of embodiments 66 to 106 wherein from 30 to 45 weight-%, preferably from 30 to 40 weight %, more preferably 30 weight % of the solid pharmaceutical composition consist of sofosbuvir based on the total weight of the solid pharmaceutical composition.
108. The process of any one of embodiments 66 to 107, wherein from 3 to 15 weight-%, more preferably from 3 to 13 weight-%, more preferably 3 to 5 weight-% of the solid pharmaceutical composition consist of the at least one pharmaceutically acceptable matrix compound based on the total weight of the solid pharmaceutical composition.
109. The process of any one of embodiments 66 to 108, wherein the solid pharmaceutical composition comprises less than 0.1 weight-%, preferably less than 0.01 weight-%, more preferably less than 0.001 weight-% of a surfactant.
110. The process of any one of embodiments 66 to 109 wherein the solid pharmaceutical composition is a solid dispersion.
111. The process of any one of embodiments 66 to 110, wherein the solid pharmaceutical composition is an oral dosage form, preferably a tablet.
112. The process of any one of embodiments 66 to 111, wherein the at least one matrix compound is selected from the group consisting of hydrophilic water-soluble polymers, silicon-based inorganic adsorbents and a combination of two or more thereof.
113. The process of any one of embodiment 66 to 112, wherein the one or more pharmaceutically acceptable excipient(s) are different from the at least one pharmaceutically acceptable matrix compound comprised in the solid pharmaceutical composition, preferably wherein the one or more pharmaceutically acceptable excipient(s) are not a pharmaceutically acceptable matrix compound as defined in any of embodiment 3 to 8.
114. The process of any of embodiments 66 to 113, wherein the one or more pharmaceutically acceptable excipient(s) includes one or more of at least one of a diluent, at least one disintegrant, at least one glidant, optionally at least one lubricant, and a combination of two or more thereof.
115. The process of embodiment 66 to 114, wherein the one or more pharmaceutically acceptable excipient(s) preferably comprise, more preferably are, a combination of at least one a diluent, at least one disintegrant, at least one glidant, and at least one lubricant.
116. The process of embodiment 115, wherein the at least one diluent includes one or more of calcium carbonate, dicalcium phosphate, dry starch, calcium sulfate, cellulose, compressible sugars, confectioner's sugar, dextrates, dextrin, dextrose, dibasic calcium phosphate dihydrate, glyceryl palmitostearate, hydrogenated vegetable oil, inositol, kaolin, lactose, magnesium carbonate, magnesium oxide, maltodextrin, mannitol, microcrystalline cellulose, polymethacrylates, potassium chloride, powdered cellulose, powdered sugar, pregelatinized starch, sodium chloride, sorbitol, starch, sucrose, sugar spheres, talc, tribasic calcium phosphate, wherein the at least one disintegrant includes one or more of agar, alginic acid, bentonite, carboxymethylcellulose calcium, carboxymethylcellulose sodium, carboxymethylcellulose, cellulose, a cation exchange resin, cellulose, gums, citrus pulp, colloidal silicon dioxide, corn starch, croscarmellose sodium, crospovidone, guar gum, hydrous aluminum silicate, an ion exchange resin such as polyacrin potassium, magnesium aluminum silicate, methyl cellulose, microcrystalline cellulose, modified cellulose gum, modified corn starch, montmorillonite clay, natural sponge, polyacrilin potassium, potato starch, powdered cellulose, povidone, pregelatinized starch, sodium alginate, sodium bicarbonate optionally in admixture with one or more acidulants, sodium starch glycolate, starch, silicates, wherein the at least one glidant includes one or more of colloidal silicon dioxide, talc, starch, starch derivatives, wherein the at least one lubricant includes one or more of calcium stearate, glyceryl monostearate, glyceryl palmitostearate, hydrogenated castor oil, hydrogenated vegetable oil, light mineral oil, magnesium stearate, mineral oil, polyethylene glycol, sodium benzoate, sodium lauryl sulfate, sodium stearyl fumarate, stearic acid, talc, zinc stearate,
117. The process of any one of embodiments 66 to 116, wherein the one or more pharmaceutically acceptable excipient(s) comprise, preferably are, a combination of mannitol, microcrystalline cellulose, croscarmellose sodium, colloidal silica, optionally magnesium stearate.
118. The process of any one of embodiments 66 to 117, for preparing the solid pharmaceutical formulation of any one of embodiments 1 to 65.
119. The process of any one of embodiments 66 to 118, wherein the solid pharmaceutical composition is a tablet.
120. A solid pharmaceutical composition obtained or obtainable according to any one of embodiments 66 to 119.
121. A tablet obtained or obtainable according to any one of embodiments 66 to 120.
122. Use of a solid pharmaceutical composition according to any one of embodiments 1 to 65 for the preparation of a medicament for treating hepatitis C in a human.
123. A method for treating hepatitis C comprising administering a solid pharmaceutical composition according to any one of embodiments 1 to 65 to a human in need thereof.

According to a further aspect, the present invention is illustrated by the following embodiments and combinations of embodiments resulting from the given dependencies and back-references:
1′. A solid pharmaceutical composition wherein the solid pharmaceutical composition comprises sofosbuvir according to formula (I)

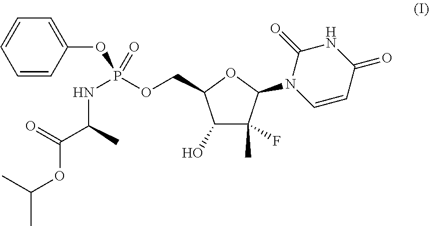 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

at least one pharmaceutically acceptable matrix compound and one or more pharmaceutically acceptable excipient(s), wherein at least 99 weight-% of the sofosbuvir comprised in the solid pharmaceutical composition are present in amorphous form, and at least 99 weight-% of the solid pharmaceutical composition consist of the sofosbuvir, the at least one matrix compound and the one or more pharmaceutically acceptable excipient(s) and wherein the at least one pharmaceutically acceptable matrix compound comprises, preferably consists of, at least one vinyl pyrrolidone-vinyl acetate copolymer.
2′. The solid pharmaceutical composition of embodiment 1′, wherein the at least one pharmaceutically acceptable matrix compound comprises, preferably consists of copovidone.
3′. The solid pharmaceutical composition of embodiment 1, wherein the solid pharmaceutical composition comprises the sofosbuvir in an amount in the range of from 15 to 95 weight-%, preferably from 20 to 70 weight-%, preferably from 20 to 45 weight-%, more preferably from 20 to 40 weight-%, more preferably 30 to 40 weight-%, more preferably 30 weight-%, based on the total weight of the solid pharmaceutical composition.
4′. The solid pharmaceutical composition of any one of embodiments 1′ to 3′, wherein at least 99.5 weight-%, preferably at least 99.9 weight-% of the sofosbuvir comprised in the composition are present in amorphous form.
5′ The solid pharmaceutical composition of any one of embodiments 1′ to 4′, wherein at least 99.5 weight-%, preferably at least 99.9 weight-% of the solid pharmaceutical composition consist of the sofosbuvir the at least one matrix compound and one or more pharmaceutically acceptable excipient(s).
6′. The solid pharmaceutical composition of any one of embodiments 1′ to 5′, wherein the solid pharmaceutical composition comprises less than 0.1 weight-%, preferably less than 0.01 weight-%, more preferably less than 0.001 weight-% of a surfactant.
7′. The solid pharmaceutical composition of any one of embodiments 1′ to 6′ wherein the solid pharmaceutical composition is a solid dispersion.
8′. The solid pharmaceutical composition of any one of embodiments 1′ to 7′, being an oral dosage form, preferably a tablet.
9′. The solid pharmaceutical composition of any one of embodiments 1′ to 8′, further comprising, in addition to the sofosbuvir, one or more further HCV agents including one or more of ledipasvir according to formula (II)

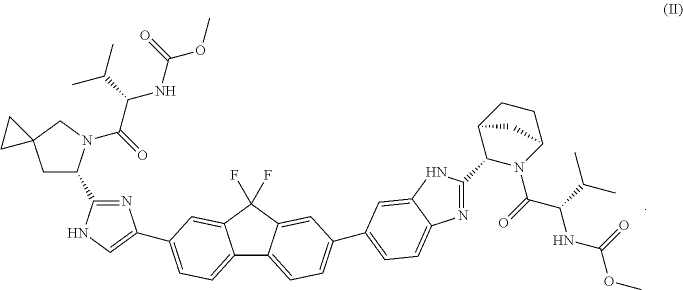 [SMILES: COC(=O)N[C@H](C(=O)N1CC2(CC2)C[C@H]1c1nc(-c2ccc3c(c2)C(F)(F)c2cc(-c4ccc5nc([C@@H]6[C@H]7CC[C@H](C7)N6C(=O)[C@@H](NC(=O)OC)C(C)C)[nH]c5c4)ccc2-3)c[nH]1)C(C)C]

10′. The solid pharmaceutical composition of any one of embodiments 1′ to 9′, wherein the solid pharmaceutical composition consists of sofosbuvir, the at least one pharmaceutically acceptable matrix compound, the one or more pharmaceutically acceptable excipient(s) and optionally the one or more further HCV agents.
11′. The solid pharmaceutical composition of any one of embodiments 1′ to 10′, wherein the one or more pharmaceutically acceptable excipient(s) comprise one or more of at least one of a diluent, at least one disintegrant, at least one glidant, optionally at least one lubricant, and combinations of two or more thereof.
12′. The solid pharmaceutical composition of embodiment 11′, wherein the at least one diluent includes one or more of calcium carbonate, dicalcium phosphate, dry starch, calcium sulfate, cellulose, compressible sugars, confectioner's sugar, dextrates, dextrin, dextrose, dibasic calcium phosphate dihydrate, glyceryl palmitostearate, hydrogenated vegetable oil, inositol, kaolin, lactose, magnesium carbonate, magnesium oxide, maltodextrin, mannitol, microcrystalline cellulose, polymethacrylates, potassium chloride, powdered cellulose, powdered sugar, pregelatinized starch, sodium chloride, sorbitol, starch, sucrose, sugar spheres, talc, tribasic calcium phosphate.
13′. The solid pharmaceutical composition of embodiment 12′, wherein the at least one diluent is mannitol.
14′. The solid pharmaceutical composition of any of embodiments 11′ to 13′, wherein the at least one disintegrant includes one or more of agar, alginic acid, bentonite, carboxymethylcellulose calcium, carboxymethylcellulose sodium, carboxymethylcellulose, cellulose, a cation exchange resin, cellulose, gums, citrus pulp, colloidal silicon dioxide, corn starch, croscarmellose sodium, crospovidone, guar gum, hydrous aluminum silicate, an ion exchange resin such as polyacrin potassium, magnesium aluminum silicate, methyl cellulose, microcrystalline cellulose, modified cellulose gum, modified corn starch, montmorillonite clay, natural sponge, polyacrilin potassium, potato starch, powdered cellulose, povidone, pregelatinized starch, sodium alginate, sodium bicarbonate optionally in admixture with one or more acidulants, sodium starch glycolate, starch, silicates.
15′. The solid pharmaceutical composition of embodiment 14′, wherein the at least one disintegrant is selected from croscarmellose sodium, such as Ac-Di-Sol® croscarmellose sodium.
16′. The solid pharmaceutical composition of any one of embodiments 11′ to 15′, wherein the at least one glidant includes one or more of colloidal silicon dioxide, talc, starch, starch derivatives.
17′. The solid pharmaceutical composition of embodiment 16′, wherein the at least one disintegrant is selected from croscarmellose sodium, such as Ac-Di-Sol® croscarmellose sodium.
18′. The solid pharmaceutical composition of any one of embodiments 11′ to 17′, wherein the at least one lubricant includes one or more of calcium stearate, glyceryl monostearate, glyceryl palmitostearate, hydrogenated castor oil, hydrogenated vegetable oil, light mineral oil, magnesium stearate, mineral oil, polyethylene glycol, sodium benzoate, sodium lauryl sulfate, sodium stearyl fumarate, stearic acid, talc, zinc stearate.
19′. The solid pharmaceutical composition of embodiment 18′, wherein the at least one glidant is colloidal silicon dioxide.
20′. The solid pharmaceutical composition of any one of embodiments 1′ to 19′, wherein the one or more pharmaceutically acceptable excipient(s) comprise, preferably are, a combination of at least one a diluent, at least one disintegrant, at least one glidant, and optionally at least one lubricant.
21′. The solid pharmaceutical composition of embodiment 20′, comprising
   from 20 to 30 weight-%, preferably from 22 to 26 weight-% diluent,
   from 20 to 30 weight-%, preferably from 24 to 28 weight-% first disintegrant,
   from 2 to 6 weight-%, preferably from 2.5 to 5.5 weight-% second disintegrant,
   from 0.5 to 7 weight-%, preferably from 0.8 to 5 weight-% glidant,
   optionally from 1 to 6 weight-%, preferably from 1.4 to 2 weight-% lubricant,
   in each based on the total weight of the solid pharmaceutical composition.
22′. The solid pharmaceutical composition of embodiment 20′ or 21′, comprising
   from 30 to 45 weight-%, preferably from 30 to 40 weight-% sofosbuvir,
   from 5 to 15 weight-%, preferably from 7 to 13 weight-% of the pharmaceutically acceptable matrix compound,
   from 20 to 30 weight-%, preferably from 22 to 26 weight-% diluent,
   from 20 to 30 weight-%, preferably from 24 to 28 weight-% first disintegrant
   from 2 to 6 weight-%, preferably from 2.5 to 5.5 weight-% second disintegrant,
   from 0.5 to 7 weight-%, preferably from 0.8 to 5 weight-% glidant,
   optionally from 1 to 6 weight-%, preferably from 1.4 to 2 weight-% lubricant,
   in each based on the total weight of the solid pharmaceutical composition.
23′. The solid pharmaceutical composition of any one of embodiments 20′ to 22′, wherein the one or more pharmaceutically acceptable excipient(s) comprise, preferably are, a combination of mannitol, microcrystalline cellulose, croscarmellose sodium, silica dioxide, magnesium stearate.
24′. The solid pharmaceutical composition of embodiment 23′, comprising
   from 20 to 30 weight-%, preferably from 22 to 26 weight-% mannitol,
   from 20 to 30 weight-%, preferably from 24 to 28 weight-% microcrystalline cellulose,
   from 2 to 6 weight-%, preferably from 2.5 to 5.5 weight-% croscarmellose sodium,
   from 0.5 to 7 weight-%, preferably from 0.8 to 5 weight-% silica dioxide,
   optionally from 1 to 6 weight-%, preferably from 1.4 to 2 weight-% magnesium stearate,
   in each based on the total weight of the solid pharmaceutical composition.
25′. The solid pharmaceutical composition of embodiment 24′, comprising
   from 30 to 45 weight-%, preferably from 30 to 40 weight-% sofosbuvir,
   from 5 to 15 weight-%, preferably from 7 to 13 weight-% copovidone,
   from 20 to 30 weight-%, preferably from 22 to 26 weight-% mannitol,
   from 20 to 30 weight-%, preferably from 24 to 28 weight-% microcrystalline cellulose,
   from 2 to 6 weight-%, preferably from 2.5 to 5.5 weight-% croscarmellose sodium,
   from 0.5 to 7 weight-%, preferably from 0.8 to 5 weight-% silica dioxide,
   optionally from 1 to 6 weight-%, preferably from 1.4 to 2 weight-% magnesium stearate,
   in each based on the total weight of the solid pharmaceutical composition.
26′. The solid pharmaceutical composition of embodiment 27′, comprising
   from 30 to 40 weight-% sofosbuvir,
   from 7 to 13 weight-% copovidone,
   from 22 to 26 weight-% mannitol,
   from 24 to 28 weight-% microcrystalline cellulose,
   from 2.5 to 5.5 weight-% croscarmellose sodium,
   from 0.8 to 5 weight-% colloidal silica,
   optionally from 1.4 to 2 weight-% magnesium stearate,
   in each based on the total weight of the solid pharmaceutical composition, wherein the individual contents add up to 100%.
27′. The solid pharmaceutical composition of any one of embodiments 1′ to 26′ which is a tablet or a coated tablet.
28′. The solid pharmaceutical composition of embodiment 27′, wherein the tablet has a weight in the range of from 900 mg to 1300 mg
29′. The solid pharmaceutical composition of any one of embodiments 1′ to 28′, obtainable or obtained by a process comprising
   (i) preparing a mixture comprising sofosbuvir, at least one pharmaceutically acceptable matrix compound and one or more pharmaceutically acceptable excipient(s), wherein at least 99 weight-% of the mixture consist of the sofosbuvir, the at least one matrix compound and the one or more pharmaceutically acceptable excipient(s);
   (ii) processing the mixture obtained from (i) to the solid pharmaceutical composition, wherein preferably the solid pharmaceutical composition is a tablet.
30′ The solid pharmaceutical composition of embodiment 29′, said process comprising (ii) directly processing the mixture obtained from (i) to the pharmaceutical composition.
31′ The solid pharmaceutical composition of embodiment 29′ or 30′ wherein (ii) comprise one or more of wet granulation, dry granulation, compression, melting extrusion of the mixture of (i).
32′. The solid pharmaceutical composition of any one of embodiments 29′ to 31′, wherein (ii) comprise melting extruding the mixture of (i), preferably hot melting extruding the mixture of (i).
33′. The solid pharmaceutical composition of any one of embodiments 1′ to 32′ wherein the sofosbuvir before melt extrusion is sofosbuvir in at least one crystalline form or in amorphous form or as a mixture of at least one crystalline form and amorphous form, wherein preferably at least 95 weight-%, preferably at least 99 weight-%, more preferably at least 99.9 weight-% of the sofosbuvir are present in at least one crystalline form, preferably crystalline form is form 1.
34′. The solid pharmaceutical composition of any one of embodiments 1 to 33′ wherein at least 99 weight-%, preferably at least 99.5 weight-%, more preferably at least 99.9 weight-% of the sofosbuvir comprised in the solid pharmaceutical composition are present in amorphous form.
35′. The solid pharmaceutical composition of any one of embodiments 1′ to 34′, wherein the melt extrusion is carried out at a temperature of at least 100° C., preferably at least 150° C.
36′. The solid pharmaceutical composition of any one of embodiments 1′ to 35′ for use in a method for treating hepatitis C in a human.
37′. The solid pharmaceutical composition of any one of embodiments 1′ to 36′ for treating hepatitis C in a human.
38′. A process for preparing a solid pharmaceutical composition according to any one of embodiments 1′ to 37′, comprising
   (i) preparing a mixture comprising sofosbuvir, at least one pharmaceutically acceptable matrix compound and one or more pharmaceutically acceptable excipient(s), wherein at least 99 weight-% of the mixture consist of the sofosbuvir, the at least one matrix compound and the one or more pharmaceutically acceptable excipient(s);
   (ii) processing the mixture obtained from (i) to the solid pharmaceutical composition.
39′. The process of embodiment 38′, said processing comprising
   (ii) directly processing the mixture obtained from (i) to the pharmaceutical composition.
40′. The process of embodiment 38′ or 39′, wherein (ii) comprise one or more of wet granulation, dry granulation, compression, melting extrusion of the mixture of (i).
41′. The process of any one of embodiments 38′ to 40′, wherein (ii) comprise melting extruding the mixture of (i), preferably hot melting extruding the mixture of (i).
42′. The process of any one of embodiments 38′ to 41′, wherein the solid pharmaceutical composition is a solid dispersion.
43′. The process of any one of embodiments 1′, wherein in (i) the weight ratio of the sofosbuvir relative to the at least one matrix compound is at least 10:1, preferably in the range of from 6:1 to 1:1 more preferably in the range of from 5:1 to 2:1, more preferably in the range of from 4.5:1 to 2.6:1.
44′. The process of any one of embodiments 40′ to 43′, wherein (ii) comprises melting extruding at a temperature in the range of from 75 to 175° C., preferably in the range of from 90 to 150° C.
45′. The process of any one of embodiments 40′ to 44′ wherein the process after the melting extruding according to (ii), comprises cooling, preferably to a temperature in the range of from 10 to 40° C., preferably in the range of from 20 to 30° C.
46′. The process of any one of embodiments 38′ to 45′, wherein the sofosbuvir before processing, preferably melting extruding is sofosbuvir in at least one crystalline form or in amorphous form or as a mixture of at least one crystalline form and amorphous form, wherein preferably at least 95 weight-%, preferably at least 99 weight-%, more preferably at least 99.9 weight-% of the sofosbuvir are present in at least one crystalline form.
47′ The process of any one of embodiments 38′ to 46′, wherein at least 99 weight-%, preferably at least 99.5 weight-%, more preferably at least 99.9 weight-% of the sofosbuvir comprised in the solid pharmaceutical composition are present in amorphous form directly after preparing the solid composition.
48′. The process of any one of embodiments 38′ to 47′ wherein the at least one pharmaceutically acceptable matrix compound comprises, preferably consists of, at least one vinyl pyrrolidone-vinyl acetate copolymer.
49′. The process of any one of embodiments 38′ to 48′, wherein the at least one hydrophilic water-soluble polymer comprises, preferably consists of, copovidone.
50′. The process of any one of embodiments 38′ to 49′ wherein at least 99.5 weight-%, preferably at least 99.9 weight-% of the sofosbuvir comprised in the composition are present in amorphous form.
51′. The process of any one of embodiments 38′ to 50′ wherein at least 99.5 weight-%, preferably at least 99.9 weight-% of the solid pharmaceutical composition consist of the sofosbuvir the at least one matrix compound and one or more pharmaceutically acceptable excipient(s).
52′. The process of any one of embodiments 38′ to 51′, wherein from 15 to 95 weight-%, preferably from 30 to 45 weight-%, more preferably from 30 to 40 weight-% of the solid pharmaceutical composition consist of sofosbuvir based on the total weight of the solid pharmaceutical composition.
53′. The process of any one of embodiments 38′ to 52′, wherein from 3 to 15 weight-%, more preferably from 3 to 13 weight-%, more preferably 3 to 5 weight-% of the solid pharmaceutical composition consist of the at least one pharmaceutically acceptable matrix compound based on the total weight of the solid pharmaceutical composition.
54′. The process of any one of embodiments 38′ to 53′, wherein the solid pharmaceutical composition comprises less than 0.1 weight-%, preferably less than 0.01 weight-%, more preferably less than 0.001 weight-% of a surfactant.
55′. The process of any one of embodiments 38′ to 54′, wherein the solid pharmaceutical composition is a solid dispersion.
56′. The process of any one of embodiments 38′ to 55′, wherein the solid pharmaceutical composition is an oral dosage form, preferably a tablet.
57′. The process of embodiment 38′ to 56′ wherein the one or more pharmaceutically acceptable excipient(s) includes one or more of at least one of a diluent, at least one disintegrant, at least one glidant, optionally at least one lubricant, and a combination of two or more thereof.
58′. The process of embodiment 38′ to 57′ wherein the one or more pharmaceutically acceptable excipient(s) preferably comprise, more preferably are, a combination of at least one a diluent, at least one disintegrant, at least one glidant, and at least one lubricant.
59′. The process of embodiment 58′, wherein the at least one diluent includes one or more of calcium carbonate, dicalcium phosphate, dry starch, calcium sulfate, cellulose, compressible sugars, confectioner's sugar, dextrates, dextrin, dextrose, dibasic calcium phosphate dihydrate, glyceryl palmitostearate, hydrogenated vegetable oil, inositol, kaolin, lactose, magnesium carbonate, magnesium oxide, maltodextrin, mannitol, microcrystalline cellulose, polymethacrylates, potassium chloride, powdered cellulose, powdered sugar, pregelatinized starch, sodium chloride, sorbitol, starch, sucrose, sugar spheres, talc, tribasic calcium phosphate, wherein the at least one disintegrant includes one or more of agar, alginic acid, bentonite, carboxymethylcellulose calcium, carboxymethylcellulose sodium, carboxymethylcellulose, cellulose, a cation exchange resin, cellulose, gums, citrus pulp, colloidal silicon dioxide, corn starch, croscarmellose sodium, crospovidone, guar gum, hydrous aluminum silicate, an ion exchange resin such as polyacrin potassium, magnesium aluminum silicate, methyl cellulose, microcrystalline cellulose, modified cellulose gum, modified corn starch, montmorillonite clay, natural sponge, polyacrilin potassium, potato starch, powdered cellulose, povidone, pregelatinized starch, sodium alginate, sodium bicarbonate optionally in admixture with one or more acidulants, sodium starch glycolate, starch, silicates, wherein the at least one glidant includes one or more of colloidal silicon dioxide, talc, starch, starch derivatives, wherein the at least one lubricant includes one or more of calcium stearate, glyceryl monostearate, glyceryl palmitostearate, hydrogenated castor oil, hydrogenated vegetable oil, light mineral oil, magnesium stearate, mineral oil, polyethylene glycol, sodium benzoate, sodium lauryl sulfate, sodium stearyl fumarate, stearic acid, talc, zinc stearate.
59′. The process of any one of embodiments 38′ to 58′ wherein the one or more pharmaceutically acceptable excipient(s) comprise, preferably are, a combination of mannitol, microcrystalline cellulose, croscarmellose sodium, colloidal silica, optionally magnesium stearate.
60′. The process of any one of embodiments 38′ to 59′ for preparing the solid pharmaceutical formulation of any one of embodiments 1 to 37′.
61′. The process of any one of embodiments 38′ to 60′, wherein the solid pharmaceutical composition is a tablet.
62′. A solid pharmaceutical composition obtained or obtainable according to any one of embodiments 38′ to 61′
63′. A tablet obtained or obtainable according to any one of embodiments 38′ to 61′.
64′. Use of a solid pharmaceutical composition according to any one of embodiments 1′ to 37′ and 62′ for the preparation of a medicament for treating hepatitis C in a human.
65′. A method for treating hepatitis C comprising administering a solid pharmaceutical composition according to any one of embodiments 1′ to 37′ and 62′ to a human in need thereof.
66′. The solid pharmaceutical composition of any one of embodiments 1′ to 37′ obtained or obtainable by melt extrusion, preferably by hot-melt extrusion.

According to a further aspect, the present invention is illustrated by the following embodiments and combinations of embodiments resulting from the given dependencies and back-references:
1*. A solid pharmaceutical composition wherein the solid pharmaceutical composition comprises sofosbuvir according to formula (I)

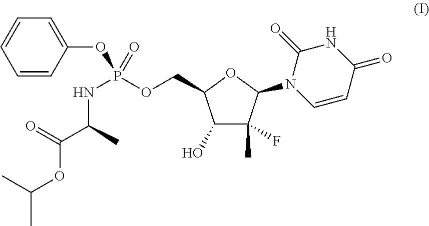 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

and one or more pharmaceutically acceptable excipient(s), wherein at least 99 weight-% of the sofosbuvir comprised in the solid pharmaceutical composition are present in amorphous form, and at least 99 weight-% of the solid pharmaceutical composition consist of the sofosbuvir and the one or more pharmaceutically acceptable excipient(s).
2*. The solid pharmaceutical composition of embodiment 1* wherein at least 99.5 weight-%, preferably at least 99.9 weight-% of the sofosbuvir comprised in the composition are present in amorphous form.
3*. The solid pharmaceutical composition of embodiment 1* or 2*, wherein the solid pharmaceutical composition comprises sofosbuvir in an amount in the range of from 15 to 95 weight-%, preferably from 20 to 70 weight-%, preferably from 20 to 45 weight-%, more preferably from 20 to 40 weight-%, more preferably 30 to 40 weight-%, more preferably 30 weight-%, based on the total weight of the solid pharmaceutical composition.
4*. The solid pharmaceutical composition of any one of embodiments 1* to 3*, wherein from 20 to 45 weight-%, more preferably from 20 to 40 weight-%, more preferably 30 to 40 weight-%, more preferably 30 weight-%, based on the total weight of the solid pharmaceutical composition.
5*. The solid pharmaceutical composition of any one of embodiments 1* to 4*, wherein 30 or 35 or 40 or 45 weight-% of the solid pharmaceutical composition consist of sofosbuvir based on the total weight of the solid pharmaceutical composition.
6*. The solid pharmaceutical composition of any one of embodiments 1* to 5*, wherein at least 99.5 weight-%, preferably at least 99.9 weight-% of the solid pharmaceutical composition consist of the sofosbuvir and one or more pharmaceutically acceptable excipient(s).
7*. The solid pharmaceutical composition of any one of embodiments 1* to 6*, being an oral dosage selected from the group consisting of a granule, a capsule such as a capsule filled with granules, a sachet, a pellet, a dragee, a lozenge, a troche, a pastille, or a tablet, such as an uncoated tablet, a coated tablet, an effervescent tablet, a soluble tablet, a dispersible tablet, an orodispersible tablet, a tablet for use in the mouth, a chewable tablet and an extrudate, preferably the solid pharmaceutical composition is a tablet or a coated tablet.
8*. The solid pharmaceutical composition of any one of embodiments 1* to 7*, further comprising, in addition to the sofosbuvir, one or more further HCV agents including one or more of ledipasvir according to formula (II)

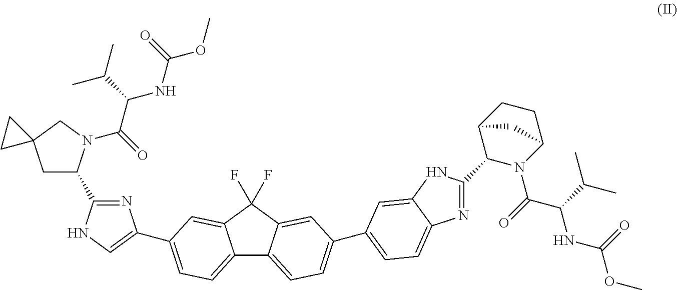 [SMILES: COC(=O)N[C@H](C(=O)N1CC2(CC2)C[C@H]1c1nc(-c2ccc3c(c2)C(F)(F)c2cc(-c4ccc5nc([C@@H]6[C@H]7CC[C@H](C7)N6C(=O)[C@@H](NC(=O)OC)C(C)C)[nH]c5c4)ccc2-3)c[nH]1)C(C)C]

9*. The solid pharmaceutical composition of any one of embodiments 1* to 8*, wherein the solid pharmaceutical composition consists of sofosbuvir, and the one or more pharmaceutically acceptable excipient(s) and optionally the one or more further HCV agents.
10*. The solid pharmaceutical composition of any one of embodiments 1* to 9*, wherein the one or more pharmaceutically acceptable excipient(s) comprise one or more of at least one diluent, of at least one disintegrant, of at least one glidant, of at least one lubricant and optionally of at least one coating agent, and combinations of two or more thereof.
11*. The solid pharmaceutical composition of embodiment 10*, wherein the at least one diluent includes one or more of calcium carbonate, dicalcium phosphate, dry starch, calcium sulfate, cellulose, compressible sugars, confectioner's sugar, dextrates, dextrin, dextrose, dibasic calcium phosphate dihydrate, glyceryl palmitostearate, hydrogenated vegetable oil, inositol, kaolin, lactose, magnesium carbonate, magnesium oxide, maltodextrin, mannitol, microcrystalline cellulose, polymethacrylates, potassium chloride, powdered cellulose, powdered sugar, pregelatinized starch, sodium chloride, sorbitol, starch, sucrose, sugar spheres, talc, tribasic calcium phosphate.
12*. The solid pharmaceutical composition of embodiment 11*, wherein the at least one diluent is selected from the group consisting of mannitol and microcrystalline cellulose or mixture thereof, wherein mannitol is preferably dry mannitol.
13*. The solid pharmaceutical composition of any one of embodiments 10* to 12*, wherein the at least one disintegrant includes one or more of agar, alginic acid, bentonite, carboxymethylcellulose calcium, carboxymethylcellulose sodium, carboxymethylcellulose, cellulose, a cation exchange resin, cellulose, gums, citrus pulp, colloidal silicon dioxide, corn starch, croscarmellose sodium, guar gum, hydrous aluminum silicate, an ion exchange resin such as polyacrin potassium, magnesium aluminum silicate, methyl cellulose, microcrystalline cellulose, modified cellulose gum, modified corn starch, montmorillonite clay, natural sponge, polyacrilin potassium, potato starch, powdered cellulose, povidone, pregelatinized starch, sodium alginate, sodium bicarbonate optionally in admixture with one or more acidulants, sodium starch glycolate, starch, silicates.
14*. The solid pharmaceutical composition of embodiment 13*, wherein the at least one disintegrant is selected from the group consisting of colloidal silicon dioxide, croscarmellose sodium, microcrystalline cellulose, preferably is croscarmellose sodium such as Ac-Di-Sol.
15*. The solid pharmaceutical composition of any one of embodiments 10* to 14*, wherein the at least one glidant includes one or more of colloidal silicon dioxide, talc, starch, starch derivatives.
16*. The solid pharmaceutical composition of embodiment 15*, wherein the at least one glidant is silicon dioxide.
17*. The solid pharmaceutical composition of any one of embodiments 10* to 16*, wherein the at least one lubricant includes one or more of calcium stearate, glyceryl monostearate, glyceryl palmitostearate, hydrogenated castor oil, hydrogenated vegetable oil, light mineral oil, magnesium stearate, mineral oil, polyethylene glycol, sodium benzoate, sodium lauryl sulfate, sodium stearyl fumarate, stearic acid, talc, zinc stearate.
18*. The solid pharmaceutical composition of embodiment 17*, wherein the at least one glidant is magnesium stearate
19*. The solid pharmaceutical composition of any one of embodiments 10* to 18*, wherein the at least one coating agent includes one or more of from the group consisting of aqueous film coating agent.
20*. The solid pharmaceutical composition of embodiment 19*, wherein the at least one aqueous film coating agent. is selected from the group consisting of OPADRY and OPADRY II.
21*. The solid pharmaceutical composition of any one of embodiments 1* to 20*, wherein the one or more pharmaceutically acceptable excipient(s) comprise, preferably are, a combination of at least one a diluent, at least one disintegrant, at least one glidant, at least one lubricant and optionally at least one coating agent.
22*. The solid pharmaceutical composition of any one of embodiments 10* to 21*, comprising
   from 25 to 35 weight-%, preferably from 25 to 35 weight-% diluent,
   from 25 to 40 weight-%, preferably from 25 to 35 weight-% disintegrant,
   from 0.5 to 7 weight-%, preferably from 0.8 to 5 weight-% glidant,
   from 1 to 6 weight-%, preferably from 1.4 to 2 weight-% lubricant,
   in each based on the total weight of the solid pharmaceutical composition.
23*. The solid pharmaceutical composition of any one of embodiments 10* to 22*, comprising
   from 30 to 45 weight-%, preferably from 30 to 40 weight-% sofosbuvir,
   from 25 to 35 weight-%, preferably from 25 to 30 weight-% diluent,
   from 25 to 40 weight-%, preferably from 25 to 30 weight-% disintegrant,
   from 0.5 to 7 weight-%, preferably from 0.8 to 5 weight-% glidant,
   from 1 to 6 weight-%, preferably from 1.4 to 2 weight-% lubricant, and optionally
   from 1 to 3.5 preferably from 2 to 3 weight-% aqueous film coating agent,
   in each based on the total weight of the solid pharmaceutical composition.
24*. The solid pharmaceutical composition of any one of embodiments 1 to 23*, wherein the one or more pharmaceutically acceptable excipient(s) comprise, preferably are, a combination of mannitol, microcrystalline cellulose, croscarmellose sodium, silica dioxide, magnesium stearate and optionally a aqueous film coating agent such as Opadry II.
25*. The solid pharmaceutical composition of any of embodiments 1* to 24*, comprising
   from 25 to 35 weight-%, preferably from 25 to 30 weight-% mannitol,
   from 25 to 34 weight-%, preferably from 25 to 30-% weight-% microcrystalline cellulose,
   from 2 to 6 weight-%, preferably from 2.5 to 5.5 weight-% croscarmellose sodium,
   from 0.5 to 7 weight-%, preferably from 0.8 to 5 weight-% silica dioxide,
   from 1 to 6 weight-%, preferably from 1.4 to 2 weight-% magnesium stearate, and
   optionally from 1.5 to 3.5 weight-% a coating agent, preferably an aqueous film coating agent,
   in each based on the total weight of the solid pharmaceutical composition.
26*. The solid pharmaceutical composition of any one of embodiments 1* to 25*, comprising
   from 25 to 60 weight-%, preferably from 30 to 45 weight-% sofosbuvir,
   from 25 to 35 weight-%, preferably from 25 to 30 weight-% mannitol,
   from 25 to 36 weight-%, preferably from 25 to 30-% weight-% microcrystalline cellulose,
   from 2 to 6 weight-%, preferably from 2.5 to 5.5 weight-% croscarmellose sodium,
   from 0.5 to 7 weight-%, preferably from 0.8 to 5 weight-% silica dioxide,
   from 1 to 6 weight-%, preferably from 1.4 to 2 weight-% magnesium stearate, and
   optionally from 1.5 to 3.5 weight-% coating agent, preferably an aqueous film coating agent, preferably Opadry II
   in each based on the total weight of the solid pharmaceutical composition.
27*. The solid pharmaceutical composition of embodiment 25* or 26*, comprising
   from 30 to 40 weight-% sofosbuvir,
   from 26 to 29 weight-% mannitol,
   from 26 to 28 weight-% microcrystalline cellulose,
   from 3.5 to 5.5 weight-% croscarmellose sodium,
   from 0.8 to 4 weight-% colloidal silica,
   optionally from 1.4 to 2 weight-% magnesium stearate,
   optionally from 2.5 to 3.5 weight-% coating agent, preferably an aqueous film coating agent,
   in each based on the total weight of the solid pharmaceutical composition, wherein the individual contents add up to 100%.
28*. The solid pharmaceutical composition of any one of embodiments 1* to 27* which is a tablet preferably selected from the group consisting of an uncoated tablet, a coated tablet, an effervescent tablet, a soluble tablet, a dispersible tablet, an orodispersible tablet, a tablet for use in the mouth, a chewable tablet preferably the solid pharmaceutical composition is a tablet or a coated tablet
29*. The solid pharmaceutical composition of embodiments 28*, wherein the tablet is a coated tablet.
30*. The solid pharmaceutical composition of embodiments 28* or 30* wherein the oral dosage form, preferably the tablet or the coated tablet has a weight in the range of from 900 mg to 1300 mg.
31*. The solid pharmaceutical composition of any one of embodiments 1* to 30*, obtainable or obtained by a process comprising
   (i) providing amorphous sofosbuvir.
   (ii) mixing the amorphous sofosbuvir of (i) and one or more pharmaceutically acceptable excipient(s), wherein at least 99 weight-% of the mixture consist of the sofosbuvir and the one or more pharmaceutically acceptable excipient(s);
   (iii) processing the mixture obtained from (ii) to the solid pharmaceutical composition.
32*. The solid pharmaceutical composition of any one of embodiments 1* to 30*, obtainable or obtained by a process comprising
   (i) providing amorphous sofosbuvir by rapid drying, more preferably by spray drying a solution comprising sofosbuvir and one or more solvents;
   (ii) mixing the amorphous sofosbuvir of (i) and one or more pharmaceutically acceptable excipient(s), wherein at least 99 weight-% of the mixture consist of the sofosbuvir and the one or more pharmaceutically acceptable excipient(s);
   (iii) processing the mixture obtained from (ii) to the solid pharmaceutical composition.
33*. The solid pharmaceutical composition of embodiment 31* or 32* wherein (iii) preferably consists of
   (iii) directly processing the mixture obtained from (ii) to the pharmaceutical composition.
34*. The solid pharmaceutical composition of any one of embodiments 31* to 33*, wherein the mixture obtained from (ii) is processed to the pharmaceutical composition according to (iii) at most 168 h, preferably at most 72 h, more preferably at most 24 h after having been obtained from (ii), wherein during this period of time, the mixture is preferably not subjected to stress conditions of 30° C. and a relative humidity of 75%, more preferably stored under ambient conditions.
35*. The solid pharmaceutical composition of any one of embodiments 31* to 34*, wherein (iii) comprise one or more of wet granulation, dry granulation, compression, melting extrusion of the mixture of (ii).
36*. The solid pharmaceutical composition of any one of embodiments 31* to 35*, wherein (iii) comprise dry granulation.
37*. The solid pharmaceutical composition of any one of embodiments 31* to 36*, wherein (iii) further comprises coating the solid pharmaceutical composition.
38*. The solid pharmaceutical composition of any one of embodiments 31* to 37*, wherein (i) comprises
   (i′) preparing a solution of sofosbuvir and one or more solvents.
39*. The solid pharmaceutical composition of any one of embodiments 31* to 39*, wherein the one or more solvents is selected from the group consisting of an organic solvent, and a combination of two or more thereof.
40*. The solid pharmaceutical composition of embodiment 39*, wherein the organic solvent is selected from the group consisting of a C1-C2 halogenated hydrocarbon such as CH₂Cl₂, a C1-C4 alcohol, such as a C1 alcohol such as methanol, a C2 alcohol such as ethanol, a C3 alcohol such as propanol, or a C4 alcohol such as butanol; a C3-C6 ketone such as a C3 ketone such as acetone, a C4 ketone, a C5 ketone, or a C6 ketone, a C2-C6 ether such as C2 ether, a C3 ether, a C4 ether, a C5 ether, or C6 ether; a C3-C5 ester such as a C3 ester, a C4 ester, or a C5 ester such as ethylacetate, a combination of two or more thereof and a combination of one or more thereof with water.
41*. The solid pharmaceutical composition of embodiment 50*, wherein the rapid drying is spray drying.
42*. The solid pharmaceutical composition of any one of embodiments 1* to 41* for use in a method for treating hepatitis C in a human.
43*. The solid pharmaceutical composition of any one of embodiments 1* to 42* for treating hepatitis C in a human.
44*. A process for preparing a solid pharmaceutical composition according to any one of embodiments 1* to 43*, comprising
   (i) providing amorphous sofosbuvir.
   (ii) mixing the amorphous sofosbuvir of (i) and one or more pharmaceutically acceptable excipient(s), wherein at least 99 weight-% of the mixture consist of the sofosbuvir and the one or more pharmaceutically acceptable excipient(s);
   (iii) processing the mixture obtained from (ii) to the solid pharmaceutical composition.
45*. A process for preparing a solid pharmaceutical composition according to any one of embodiments 1* to 43*, comprising
   (i) providing sofosbuvir by rapid drying, preferably by spray drying a solution comprising sofosbuvir and one or more solvents,
   (ii) mixing the amorphous sofosbuvir of (i) and one or more pharmaceutically acceptable excipient(s), wherein at least 99 weight-% of the mixture consist of the sofosbuvir, and the one or more pharmaceutically acceptable excipient(s);
   (iii) processing the mixture obtained from (ii) to the solid pharmaceutical composition, wherein preferably the solid pharmaceutical composition is a tablet.
46*. The process of embodiment 44* or 45*, wherein at least 99 weight-%, preferably at least 99.5 weight-%, more preferably at least 99.9 weight-% of the sofosbuvir comprised in the solid pharmaceutical composition are present in amorphous form.
47*. The process of any of embodiments 44* to 46*, wherein (iii) preferably consists of
   (iii)) directly processing the mixture obtained from (ii) to the pharmaceutical composition.
48*. The process of any of embodiments 44* to 47*, wherein after (ii) and before (iii), the mixture obtained from (ii) is not subjected to any modification.
49*. The process of any one of embodiments 44* to 48*, wherein the mixture obtained from (ii) is processed to the pharmaceutical composition according to (iii) at most 168 h, preferably at most 72 h, more preferably at most 24 h after having been obtained from (ii), wherein during this period of time, the mixture is preferably not subjected to stress conditions of 30° C. and a relative humidity of 75%, more preferably stored under ambient conditions.
50*. The process of any one of embodiments 44* to 49*, wherein (iii) comprise one or more of wet granulation, dry granulation, compression, melting extrusion of the mixture of (ii).
51*. The process of embodiment 50*, wherein (iii) comprises direct compression or dry granulation.
52*. The process of any one of embodiments 44* to 51*, wherein (iii) further comprises coating the solid pharmaceutical composition.
53*. The process of any one of embodiments 45* to 52* wherein (i) comprises
   (i′) preparing a solution of sofosbuvir and one or more solvents.
54*. The process of embodiments 45* to 53*, wherein the one or more solvents is selected from the group consisting of an organic solvent, and a combination of two or more thereof.
55*. The process of embodiment 54*, wherein the organic solvent is selected from the group consisting of a C1-C2 halogenated hydrocarbon such as CH₂Cl₂, a C1-C4 alcohol, such as a C1 alcohol such as methanol, a C2 alcohol such as ethanol, a C3 alcohol such as propanol, or a C4 alcohol such as butanol; a C3-C6 ketone such as a C3 ketone such as acetone, a C4 ketone, a C5 ketone, or a C6 ketone; a C2-C6 ether such as C2 ether, a C3 ether, a C4 ether, a C5 ether, or C6 ether; a C3-C5 ester such as a C3 ester, a C4 ester, or a C5 ester such as ethylacetate, a combination of two or more thereof and a combination of one or more thereof with water.
56*. The process of any of embodiments 45* to 54*, wherein the rapid drying is spry drying.
57*. The process of any of embodiments 44* to 55*, wherein the solid pharmaceutical composition comprises the in an amount in the range of from 15 to 95 weight-%, preferably from 20 to 70 weight-%, preferably from 20 to 45 weight-%, more preferably from 20 to 40 weight-%, more preferably 30 to 40 weight-%, more preferably 30 weight-%, based on the total weight of the solid pharmaceutical composition.
58*. The process of any of embodiments 44* to 57*, wherein from 20 to 45 weight-%, more preferably from 20 to 40 weight-%, more preferably 30 to 40 weight-%, more preferably 30 weight-%, based on the total weight of the solid pharmaceutical composition.
59*. The process of any of embodiments 44* to 58*, wherein 30 or 35 or 40 or 45 weight-% of the solid pharmaceutical composition consist of sofosbuvir based on the total weight of the solid pharmaceutical composition.
60*. The process of any one of embodiments 44* to 59*, wherein at least 99.5 weight-%, preferably at least 99.9 weight-% of the solid pharmaceutical composition consist of the sofosbuvir and one or more pharmaceutically acceptable excipient(s).
61*. The process of any one of embodiments 44* to 60*, wherein the solid pharmaceutical composition is an oral dosage form selected from the group consisting of a granule, a capsule such as a capsule filled with granules, a sachet, a pellet, a dragee, a lozenge, a troche, a pastille, or a tablet, such as an uncoated tablet, a coated tablet, an effervescent tablet, a soluble tablet, a dispersible tablet, an orodispersible tablet, a tablet for use in the mouth, a chewable tablet and an extrudate, preferably the solid pharmaceutical composition is a tablet or a coated tablet.
62*. The process of any one of embodiments 44* to 61*, wherein the one or more pharmaceutically acceptable excipient(s) of (ii) comprise one or more of at least one of a diluent, of at least one disintegrant, of at least one glidant, of at least one lubricant and combinations of two or more thereof.
63*. The process of embodiment 62*, wherein the at least one diluent includes one or more of calcium carbonate, dicalcium phosphate, dry starch, calcium sulfate, cellulose, compressible sugars, confectioner's sugar, dextrates, dextrin, dextrose, dibasic calcium phosphate dihydrate, glyceryl palmitostearate, hydrogenated vegetable oil, inositol, kaolin, lactose, magnesium carbonate, magnesium oxide, maltodextrin, mannitol, microcrystalline cellulose, polymethacrylates, potassium chloride, powdered cellulose, powdered sugar, pregelatinized starch, sodium chloride, sorbitol, starch, sucrose, sugar spheres, talc, tribasic calcium phosphate, wherein the at least one disintegrant includes one or more of agar, alginic acid, bentonite, carboxymethylcellulose calcium, carboxymethylcellulose sodium, carboxymethylcellulose, cellulose, a cation exchange resin, cellulose, gums, citrus pulp, colloidal silicon dioxide, corn starch, croscarmellose sodium, crospovidone, guar gum, hydrous aluminum silicate, an ion exchange resin such as polyacrin potassium, magnesium aluminum silicate, methyl cellulose, microcrystalline cellulose, modified cellulose gum, modified corn starch, montmorillonite clay, natural sponge, polyacrilin potassium, potato starch, powdered cellulose, povidone, pregelatinized starch, sodium alginate, sodium bicarbonate optionally in admixture with one or more acidulants, sodium starch glycolate, starch, silicates, wherein the at least one glidant includes one or more of colloidal silicon dioxide, talc, starch, starch derivatives, wherein the at least one lubricant includes one or more of calcium stearate, glyceryl monostearate, glyceryl palmitostearate, hydrogenated castor oil, hydrogenated vegetable oil, light mineral oil, magnesium stearate, mineral oil, polyethylene glycol, sodium benzoate, sodium lauryl sulfate, sodium stearyl fumarate, stearic acid, talc, zinc stearate,
64*. The process of any one of embodiments 44* to 63*, wherein the one or more pharmaceutically acceptable excipient(s) of (ii) comprise, preferably are, a combination of mannitol, microcrystalline cellulose, croscarmellose sodium, colloidal silica and magnesium stearate.
65*. The process of any one of embodiments 44* to 64* for preparing the solid pharmaceutical formulation of any one of embodiments 1* to 43*.
66*. A solid pharmaceutical composition obtained or obtainable according to any one of embodiments 44* to 65*.
67*. A tablet obtained or obtainable by a process according to any one of embodiments 44* to 66*.
68*. Use of a solid pharmaceutical composition according to any one of embodiments 1* to 43* for the preparation of a medicament for treating hepatitis C in a human.
69*. A method for treating hepatitis C comprising administering a solid pharmaceutical composition according to any one of embodiments 1* to 43* or 66* to a human in need thereof.
70*. A process for preparing amorphous sofosbuvir comprising spray drying sofosbuvir.

The present invention is further illustrated by the following reference examples and examples.

### EXAMPLES

### Reference Example 1: Dynamic Vapor Sorption (DVS) Measurements—Determination of Δm(Desorption) and Δm(Adsorption) at 75% Relative Humidity and 25° C.

The adsorption-desorption isotherms from which the values of Δm(desorption) and Δm(adsorption) at 75% relative humidity and at 25° C. were obtained, were recorded with an SPSx-1μ (1 micro) moisture sorption analyzer (ProUmid GmbH & Co. KG, Ulm, Germany). A given measurement cycle was started at ambient relative humidity (r.h.), in the present case 40% r.h. The r.h. was decreased to 3% and then to 0%. For this isotherm, as black filled square with a white x inside is used in the respective Figures. Subsequently, the adsorption isotherm (symbols: ▪ in the Figures) was recorded, i.e. r.h. was increased to 5%, then to 10%, and thereafter in 10% steps. Once having reached the chosen maximum r.h. value, the desorption isotherm (symbols: ● in the Figures) was recorded, starting with 10% steps down to a r.h. of 10%, followed by a r.h. decrease in 5% steps to 0% r.h. The last step consisted of increasing the r.h. to ambient r.h. As to the isotherm obtained by the last step, a black filled square with a white asterisk inside is used as symbol in the respective Figures. The time per step was set to 3 to 5 hours. For all steps and all isotherms, the temperature was set to 25±0.1° C. To obtain the Δm(desorption) and Δm(adsorption) values, the recorded adsorption-desorption isotherms shown in the Figures of the present invention were analysed by comparing the value of Δm(desorption), plotted on the y axis, of a given desorption isotherm with the value of Δm(adsorption), plotted on the y axis, of the respective adsorption isotherm, both at 75% r.h., plotted on the x axis.

### Reference Example 2: Determination of the Moisture Stability

25-30 mg of a given solid composition prepared according to the Examples below were exposed to an atmosphere having a relative humidity of 75% and a temperature of 40° C. for 8 weeks or more and analysed via XRD as described in Reference Example 3 with respect to the amorphousness. In example 2 the sample deliquescences after 2 weeks.

### Reference Example 3: XRPD Measurements

The X-ray powder diffraction patterns (XRPD) were obtained with a PANalytical X'Pert PRO diffractometer equipped with a theta/theta coupled goniometer in transmission geometry, programmable XYZ stage with well plate holder, Cu-Kalpha1,2 radiation (wavelength 0.15419 nm) with a focusing mirror and a solid state PIXcel detector. The patterns were recorded at a tube voltage of 45 kV and a tube current of 40 mA, applying a step size of 0.013° 2-theta with 40 s per step (255 channels) in the angular range of 2° to 40° 2-theta at ambient conditions.

### Example 1: Preparation of a Solid Pharmaceutical Composition Comprising Amorphous Sofosbuvir

5.000 mg sofosbuvir crystalline form 1 prepared according to WO 2011/123645 A, Example 10, and 1.125 mg Kollidon® VA 64 Fine (a vinyl pyrrolidone vinyl acetate copolymer; from BASF SE; ΔΔm=−0.09%; the DVS isotherm of this matrix compound is shown in FIG. 1), were dry-mixed. The obtained dry mixture was subjected to hot-melt extrusion at (120±30) ° C. using a DSM-Explore V micro compounder. The obtained extrudate was cooled to ambient temperature. The cooled extrudate was then crushed using a sieve having a mesh size of 0.5 mm. Through a sieve having a mesh size of 0.5 mm, the components and the respective amounts as given in the following table 1 were admixed to obtain a respective mixture. The solid composition was used 168 h after having been prepared, after storage under ambient conditions.

TABLE 1
Mixture according to Example 1

| Component | Amount/mg |
|---|---|
| Solid composition | 4.90 |
| Mannitol (from Merck) | 2.50 |
| Microcrystalline Cellulose (Avicel ® 102) | 3.71 |
| Crosscarmellose Sodium (Ac-Di-Sol ® Typ A) | 0.60 |
| Colloidal Silica (Aerosil ® 200) | 0.11 |
| Magnesium Stearate (from Fati) | 0.18 |
| Total Amount: | 12.00 |

The obtained mixture was processed to oblong tablets having a geometry of 20 mm×9 mm and a mass of 1.20 g using a FlexiTab apparatus. The pressure used for preparing the tablets was 14.1 kN. Then, the solid pharmaceutical composition was subjected to an atmosphere having a relative humidity of 75% and a temperature of 40° C. for 8 weeks as described in Reference Example 2. It was found that, although amorphous sofosbuvir was not stabilized in the solid composition above, the amorphous sofosbuvir comprised in the solid composition directly after its preparation was stable in the finally obtained solid pharmaceutical composition. The respective XRDP is shown in FIG. 2, upper Figure. Clearly, comparing the upper Figure of FIG. 2 with the two XRPDs of the 2 different crystalline forms of mannitol in the middle and lower Figure, one notes that apart from mannitol, no further crystalline compound is present in the tablet and that, therefore, the sofosbuvir comprised in the tablet is present completely in amorphous form.

### Example 2: Preparation of a Solid Dispersion Comprising Amorphous Sofosbuvir and Kollidon® VA64

200 mg sofosbuvir crystalline Form 1 (prepared according to WO 2011/123645 A, Example 10) and 45 mg Kollidon® VA64 (a vinyl pyrrolidone vinyl acetate copolymer commercially available from BASF SE) were dissolved in 4 mL ethanol, followed by addition of 12 mL DI water. The homogeneous solution was frozen in a bath of liquid nitrogen and lyophilized at a temperature of from −40° C. to −30° C. at a pressure of from 0 to 2 mbar, yielding an amorphous solid dispersion. The X-ray powder diffractogram (XRPD) of the solid dispersion is shown in FIG. 3.

The resulting solid dispersion was subjected to a moisture stability test according to Reference Example 2, leading to sample deliquescence. After two weeks upon storage at a relative humidity of 75% and a temperature of 40° C. a transparent gel was obtained. As shown by the data amorphous sofosbuvir was not stabilized in the solid composition of Example 2. However amorphous sofosbuvir comprised in this solid composition directly after its preparation is stable in the finally obtained solid pharmaceutical composition.

### Example 3: Preparation of a Solid Pharmaceutical Composition Comprising Amorphous Sofosbuvir: Hot Melt Extrusion

400 mg sofosbuvir crystalline form 1 prepared according to WO 2011/123645 A, Example 10. 90 mg Kollidon® (a vinyl pyrrolidone vinyl acetate copolymer; from BASF SE), 270 mg of mannitol powder, 300 mg of cellulose microcrystalline, 30 mg of croscarmellose sodium and 54 mg of colloidal silica dioxide were dry-mixed and homogenized.

The obtained dry mixture was subjected to hot-melt extrusion at (120±30) ° C. using a DSM-Explore V micro compounder. The obtained extrudate was cooled to ambient temperature (25° C.). The cooled extrudate was then crushed using a sieve having a mesh size of 1.5 mm.

The extrudate was further mixed with 60 mg of Cellulose Microcrystalline, 30 mg Croscarmellose sodium 6 mg colloidal silicon dioxide and 9 mg of Magnesium stearate. The mixture was further tableted by direct compression.

The tablet was coated with Opadry II

TABLE 3
Mixture according to Example 3

| | Amount in | |
|---|---|---|
| Component | mg | 100% |
| Sofosbuvir | 400 | 38.85 |
| Kollidon VA 64 Fine ® | 90 | 8.74 |
| Mannitol | 270 | 26.23 |
| Microcrystalline Cellulose Ep/Nf, Grob | 300 | 29.14 |
| Crosscarmellose Sodium | 30 | 2.91 |
| (Ac-Di-Sol ® Typ A) | | |
| Silica dioxide | 54 | 0.52 |
| Extrudate | 1095 | 88.67 |
| Microcrystalline Cellulose Ep/Nf, Grob | 60 | 4.86 |
| Crosscarmellose Sodium | 30 | 2.43 |
| Colloidal silicon dioxide | 6 | 0.49 |
| Magnesium stearate | 9 | 0.73 |
| Opadry II | 35 | 2.83 |
| E-Water | 140 | 11.33 |

The resulting solid pharmaceutical composition comprising amorphous sofosbuvir was subjected to a moisture stability test according to Reference Example 2. Upon storage at a relative humidity of 75% and a temperature of 40° C. no change was observed. As shown in FIG. 4 the XRPD patterns were measured at 0, 1, 2 and 3 months. The corresponding XRPD patterns are reported in FIG. 4. The “a” pattern corresponds to the XRPD measured at the beginning of the stability testing, while the b, c and d patterns correspond to XRPD's measured after 1, 2 and 3 months, respectively. The peaks in the XRPD are due to mannitol, while sofosbuvir in the sample was and remained amorphous. As shown in FIG. 4 by the data, amorphous sofosbuvir was stabilized in the solid pharmaceutical composition of Example 3. The XRPD patterns were measured according to Reference Example 3.

### SHORT DESCRIPTION OF THE FIGURES

FIG. 1 in its upper part shows the DVS isotherm of the matrix compound Kollidon VA 64 Fine (Example 1), recorded as described in Reference Example 1. The x axis shows the r.h. (relative humidity, in %) values, with tick marks, from left to right, at 0,0; 10,0; 20,0; 30,0; 40,0; 50,0; 60,0; 70,0; 80,0; 90,0; and 100,0. The y axis shows the Δm values (in %), with tick marks, from bottom to top, at −10,0; 0,0; 10,0; 20,0; 30,0; 40,0; 50,0; 60,0. The Δm(desorption) values are obtained from the desorption isotherm (symbols: ●), the Δm(adsorption) values are obtained from the adsorption isotherm (symbols: ▪). At 75% r.h., the value of ΔΔm, determined as described in Reference Example 1, is −0.09. This is shown, in particular, in the lower part of FIG. 1 which is a detailed portion of the upper part of of FIG. 1.

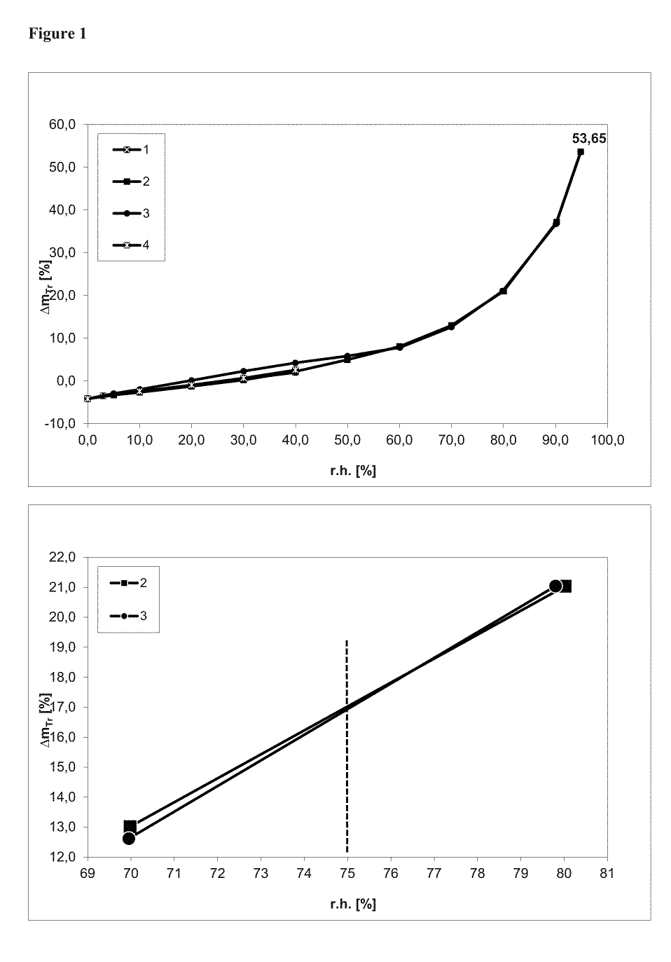

FIG. 2 shows an overlay of 3 XRPDs. In the upper figure, the XRPD of the tablet according to Example 1 after the moisture stability test according to Reference Example 1. In the middle figure, the XRPD of a first crystalline form of mannitol, comprised in the tablet, is shown. In the bottom figure, the XRPD of a second crystalline form of mannitol, comprised in the tablet, is shown. The parameters of the XRPD measurement are described in Reference Example 3. The x axis shows the 2 theta/° values, with tick marks, from left to right, at 10, 20, 30. The y axis shows the intensity in counts, with tick marks, from bottom to top, at 0, 1000, and 2000.

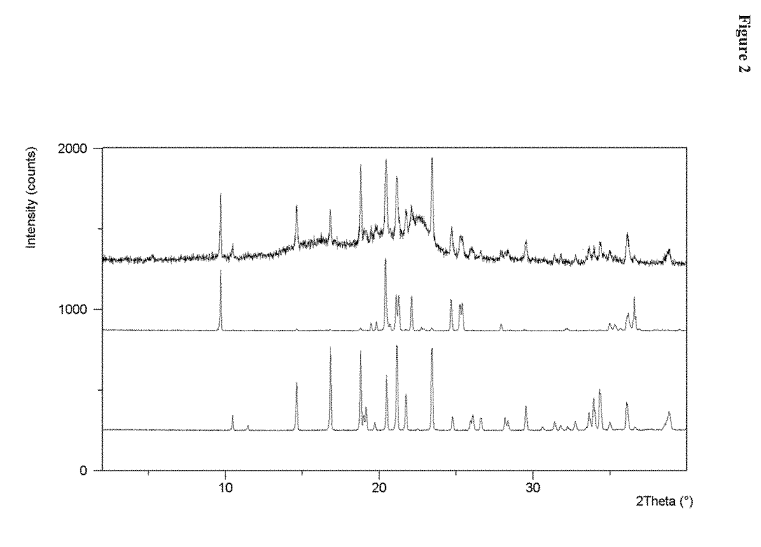

FIG. 3: Representative XRPD pattern of the solid composition prepared according to the Example 2. The x axis shows the 2 theta/° values, with tick marks, from left to right, at 10, 20, and 30. The y axis shows the intensity in counts, with tick marks, from bottom to top, at 0, 500, and 1000.

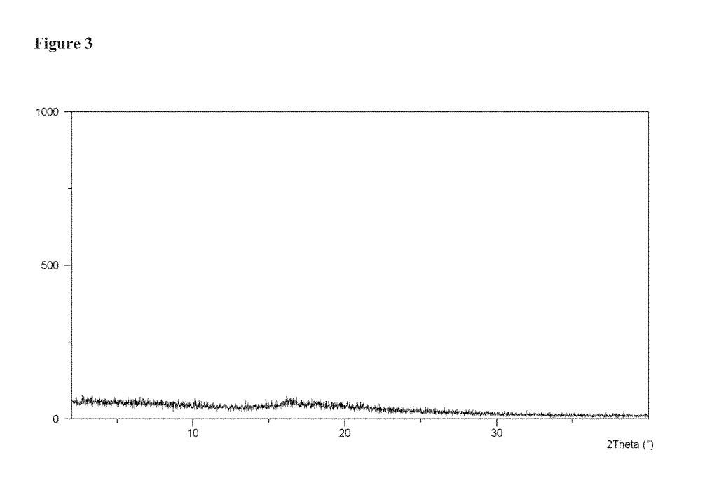

FIG. 4: Representative XRPD patterns a, b, c, and d of the solid pharmaceutical composition (tablet) prepared according to the Example 3. The XRPD patterns have been measured during the stability testing for the composition of Example 3 (3 months, 40° C., 75% r.h.). The “a” pattern corresponds to the XRPD measured at the beginning of the stability testing, while the b, c and d patterns correspond to XRPD's measured after 1, 2 and 3 months, respectively. The peaks in the XRPD are due to mannitol, analogously to what is described for Example 1 and FIG. 2, while sofosbuvir in the sample was and remained amorphous.

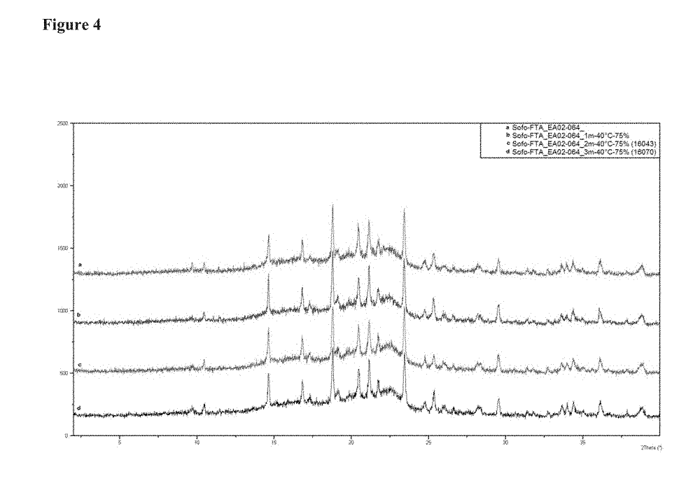

### CITED PRIOR ART

WO 2010/135569 A
WO 2013/101550 A
WO 2011/123645 A

1. A solid pharmaceutical composition comprising a solid composition, wherein the solid composition comprises sofosbuvir according to formula (I) 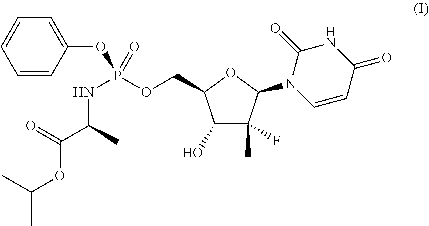 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]
and at least one pharmaceutically acceptable matrix compound, wherein at least 99 weight-% of the sofosbuvir comprised in the solid composition are present in amorphous form, at least 99 weight-% of the solid composition consists of the sofosbuvir and the at least one matrix compound, wherein the solid composition contains the sofosbuvir in an amount of at least 25 weight-%, based on the combined weight of the sofosbuvir and the at least one matrix compound,
wherein in the adsorption-desorption isotherm of the at least one pharmaceutically acceptable matrix compound, ΔΔm/% which is defined as the mass difference Δm(desorption)/% at 75% relative humidity and 25° C. minus the mass difference Δm(adsorption)/% at 75% relative humidity and 25° C., determined according to dynamic vapor sorption measurement, is <0.

2. The solid pharmaceutical composition of claim 1, wherein the solid composition comprises the sofosbuvir in an amount in the range of from 55 to 95 weight-%, based on the combined weight of the sofosbuvir and the at least one matrix compound.

3. The solid pharmaceutical composition of claim 1, wherein ΔΔm/% is in the range of from −0.1≤ΔΔm/%<0.

4. The solid pharmaceutical composition of claim 1, wherein the at least one matrix compound is selected from the group consisting of hydrophilic water-soluble polymers and combinations of two or more thereof, wherein the at least one hydrophilic water-soluble polymer comprises at least one vinyl pyrrolidone-vinyl acetate copolymer.

5. The solid pharmaceutical composition of claim 4, wherein the at least one hydrophilic water-soluble polymer comprises copovidone.

6. The solid pharmaceutical composition of claim 1, wherein the solid composition is a solid dispersion.

7. The solid pharmaceutical composition of claim 1, being a tablet.

8. The solid pharmaceutical composition of claim 1, comprising the solid composition and at least one pharmaceutically acceptable excipient.

9. The solid pharmaceutical composition of claim 8, wherein the at least one pharmaceutically acceptable excipient includes one or more of at least one of a diluent, at least one disintegrant, at least one glidant, at least one lubricant, and a combination of two or more thereof.

10. The solid pharmaceutical composition of claim 8, wherein the at least one pharmaceutically acceptable excipient comprises a combination of mannitol, microcrystalline cellulose, croscarmellose sodium, colloidal silica, magnesium stearate.

11. The solid composition of claim 1 for use in a method for treating hepatitis C in a human.

12. A process for preparing a solid pharmaceutical composition according to claim 1, comprising
(i) preparing a solid composition comprising sofosbuvir and at least one pharmaceutically acceptable matrix compound, wherein at least 99 weight-% of the sofosbuvir comprised in the solid composition are present in amorphous form, at least 99 weight-% of the solid composition consist of the sofosbuvir and the at least one matrix compound, wherein the solid composition contains the sofosbuvir in an amount of at least 25 weight-%, based on the combined weight of the sofosbuvir and the at least one matrix compound, and
(ii) processing the solid composition obtained from (i) to the pharmaceutical composition;
wherein in the adsorption-desorption isotherm of the at least one pharmaceutically acceptable matrix compound, ΔΔm/% which is defined as the mass difference Δm(desorption)/% at 75% relative humidity and 25° C. minus the mass difference Δm(adsorption)/% at 75% relative humidity and 25° C., determined according to dynamic vapor sorption measurement is <0.

13. The process of claim 12, wherein (i) comprises embedding the sofosbuvir in a matrix comprising the at least one pharmaceutically acceptable matrix compound, wherein the weight ratio of the sofosbuvir relative to the at least one matrix compound is at least 5.5:4.5.

14. The process of claim 12, wherein (ii) comprises preparing a mixture of the solid composition obtained from (i) and at least one pharmaceutically acceptable excipient and processing the mixture to a tablet.

15. (canceled)

16. The solid pharmaceutical composition of claim 1, wherein the solid composition has a moisture stability of at least 95%, wherein the moisture stability is defined as the amount of solid amorphous sofosbuvir which is present in the solid pharmaceutical composition after having been exposed to a relative humidity of 75% at 40° C. for 8 weeks, relative to the amount of solid amorphous sofosbuvir which is present in the solid pharmaceutical composition before said exposure.
